(19) United States
(12) Patent Application Publication
(10) Pub. No.: US 2017/0073690 A1
(43) Pub. Date: Mar. 16, 2017

(54) ANTAGONISTS OF SLC38A9 AND THEIR USE IN THERAPY

(71) Applicant: CEMM - Forschungzentrum Fuer Molekulare Medizin GMBH, Wien (AT)

(72) Inventors: Manuele Rebsamen, Wien (AT); Giulio Superti-Furga, Wien (AT)

(21) Appl. No.: 15/311,108

(22) PCT Filed: May 15, 2015
(86) PCT No.: PCT/EP2015/060772

§ 371 Date: Nov. 14, 2016

(30) Foreign Application Priority Data
May 15, 2014 (EP) 14168539.6

(51) Int. Cl. C12N 15/113; G01N 33/50; C12N 15/115

(52) U.S. Cl. CPC C12N 15/1138; C12N 15/115; G01N 33/5023; C12N 2310/14; C12N 2310/531; C12N 2320/30; C12N 2310/16

(57) ABSTRACT

The present invention relates to an antagonist or modulator of SLC38A9 for use in treating a disease associated with mTORC1 activation, like a proliferative disease (e.g. a cancerous disease or benign proliferative disease), a metabolic disorder, a disorder of the immune system, a disorder causing premature aging, an ophthalmic disorder or a neurological disorder. Exemplary diseases to be treated are cancerous diseases like lung cancer, breast cancer, bladder cancer, pancreatic cancer, ovarian cancer, colon carcinoma, leukemia, lymphoma, melanoma, esophageal cancer and stomach cancer; or metabolic disorders like overweight (pre-obesity), obesity or diabetes. Also provided herein are methods for treating, preventing or ameliorating such diseases comprising the administration of an antagonist of SLC38A9 to a subject in need of such a treatment, prevention or amelioration. Furthermore, the present invention provides methods for assessing the activity of a candidate molecule suspected of being an antagonist of SLC38A9 and identification of such antagonists.

The present invention relates to an antagonist or modulator of SLC38A9 for use in treating a disease associated with mTORC1 activation, like a proliferative disease (e.g. a cancerous disease or benign proliferative disease), a metabolic disorder, a disorder of the immune system, a disorder causing premature aging, an ophthalmic disorder or a neurological disorder. Exemplary diseases to be treated are cancerous diseases like lung cancer, breast cancer, bladder cancer, pancreatic cancer, ovarian cancer, colon carcinoma, leukemia, lymphoma, melanoma, esophageal cancer and stomach cancer; or metabolic disorders like overweight (pre-obesity), obesity or diabetes. Also provided herein are methods for treating, preventing or ameliorating such diseases comprising the administration of an antagonist of SLC38A9 to a subject in need of such a treatment, prevention or amelioration. Furthermore, the present invention provides methods for assessing the activity of a candidate molecule suspected of being an antagonist of SLC38A9 and identification of such antagonists.

Cell growth and proliferation are tightly linked to nutrient availability. The mechanistic target of rapamycin complex 1 (mTORC1) integrates the presence of growth factors, energy levels, glucose and amino acid availability to modulate metabolic status and cellular responses^{1,2}. mTORC1 is recruited to the surface of lysosomes by RAG GTPases and the Ragulator in dependence of amino acid availability^{3,5}. Aberrant activation of mTORC1 in known in the art to be implicated in several disease, and in particular metabolic disorders like type 2 diabetes as well as in proliferative malignancies like cancer. Compounds targeting mTORC1 (such as rapamycin and rapamycin analogs) are tested in clinical trials and some compounds have already been approved by the FDA.

The mechanism leading to activation of mTOR has been at the center of attention of investigators for several years as coupling of nutrient and growth stimuli to metabolic state and cellular fate is critically involved in a variety of diseases including cancer and metabolic disorders^{7,8}. Amino acids are essential for mTORC1 activity, as growth factors cannot efficiently activate mTOR in their absence^{4,9}. How amino acid levels are sensed is still unclear and several complementary mechanisms have been proposed¹⁰⁻¹². It is thought, however, that the levels of amino acids within the lysosomal lumen signal to the Ragulator complex via a poorly defined mechanism involving the vacuolar H⁺-ATPase¹³. The Ragulator complex tethers the RAG GTPases to the lysosome and, in presence of amino acids, acts as a guanine nucleotide exchange factor (GEF) for RAGA/B¹⁴. RAG GTPases function as heterodimers of RAGA/B with RAGC/D and in their active nucleotide loaded state, RAGA/B^{GTP}-RAGC/D^{GDP}, bind RAPTOR and recruit mTORC1 to the lysosomal surface where it is activated by RHEB. However, despite the growing number of proteins involved in the regulation of mTOR activation at the lysosomal surface the nature of the amino acid sensor has remained mysterious^{1,6,8}.

Thus, the technical problem underlying the present invention is the provision of means and methods for the medical intervention in diseases associated with aberrant mTORC1 activation.

The technical problem is solved by provision of the embodiments characterized in the claims.

The present invention relates to an antagonist of SLC38A9 for use in treating a disease associated with mTORC1 activation, in particular aberrant mTORC1 activation.

The present invention relates to a method for treating, preventing or ameliorating a disease associated with mTORC1 activation (in particular aberrant mTORC1 activation) comprising the administration of an antagonist of SLC38A9 to a subject in need of such a treatment, prevention or amelioration. Preferably, the subject or patient to be treated in accordance with the present invention is a human.

The present invention solves the above identified technical problem since, as documented herein below and in the appended examples, it was surprisingly found that the so far uncharacterized human member 9 of the solute carrier family 38 (SLC38A9, Sentor) is a lysosomal membrane resident protein competent in amino acid transport that control mTORC1 activation. Extensive functional proteomic analysis established Sentor as an integral part of the Ragulator/RAG GTPases machinery. “Antagonists of SLC38A9” to be used in accordance with the present invention and assays for identifying or validating these antagonists are described further below.

It was surprisingly found herein that inhibition of one (so far uncharacterized) protein, namely SLC38A9, (Sentor), that interacts with the Ragulator/RAG GTPase complex is sufficient for interfering with, in particular inhibiting, mTORC1 activity.

The mTOR complex 1 (mTORC1) is composed of mTOR, a serine/threonine kinase (Uniprot: P42345), Raptor (Uniprot: Q8N122); mLST8 (Uniprot: Q9BVC4), Deptor (Uniprot: Q8TB45) and PRAS40 (Uniprot: Q96B36). Exemplary nucleotide sequences and amino acid sequences of the mTOR complex 1 are shown in SEQ ID NO: 23-52. The terms “mTOR complex 1” and “mTORC1” are used interchangeably herein. Aberrant activation of mTORC1 in known in the art to be implicated in several diseases, inter alia, diabetes 2 and cancerous diseases.

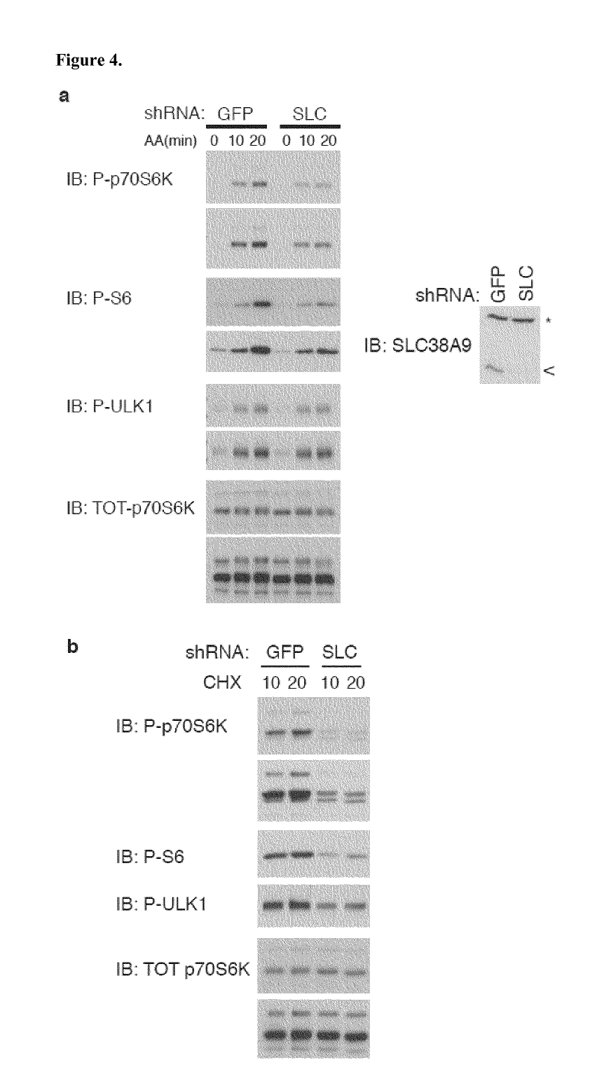

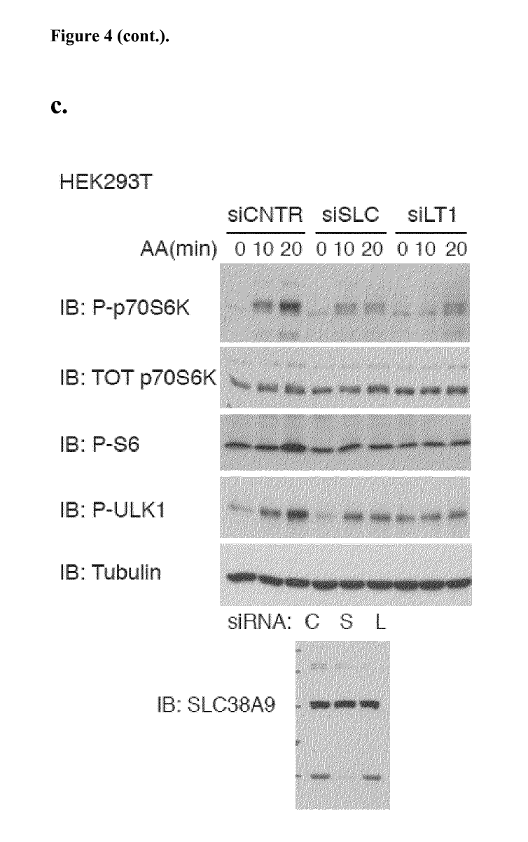

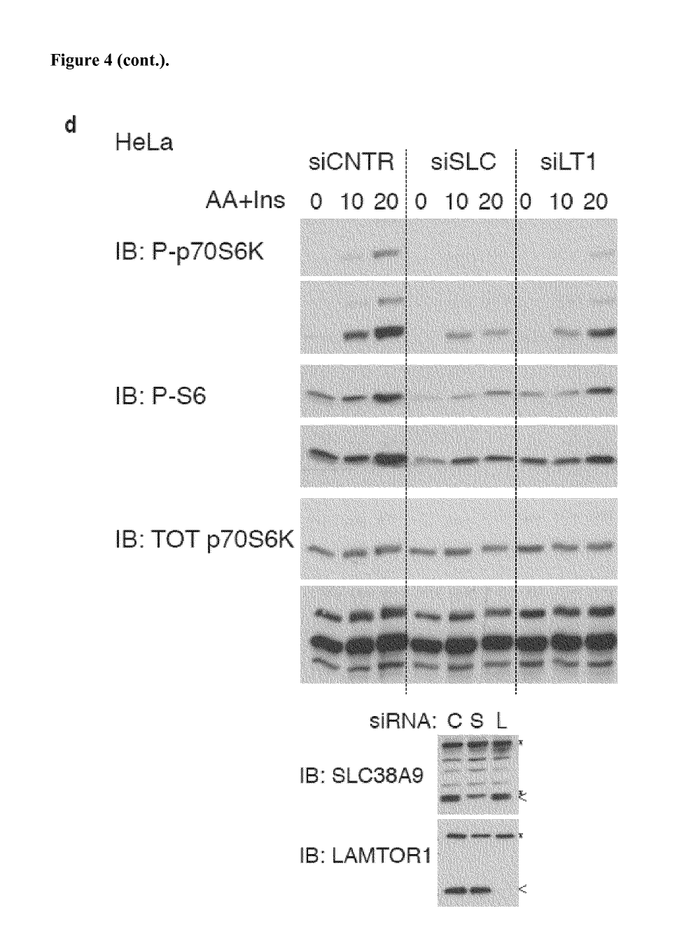

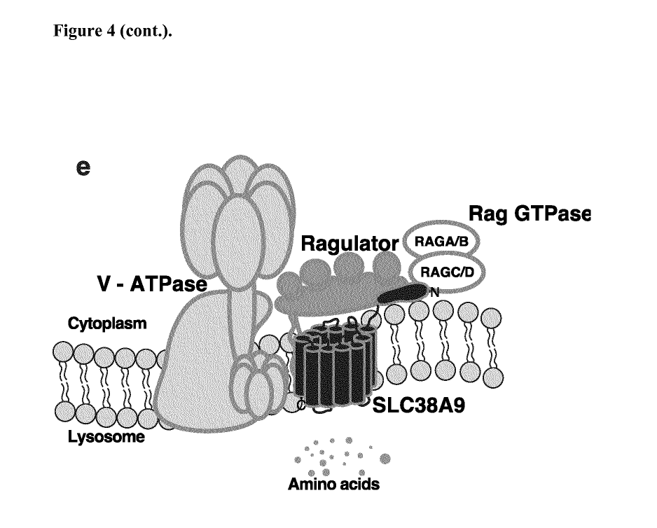

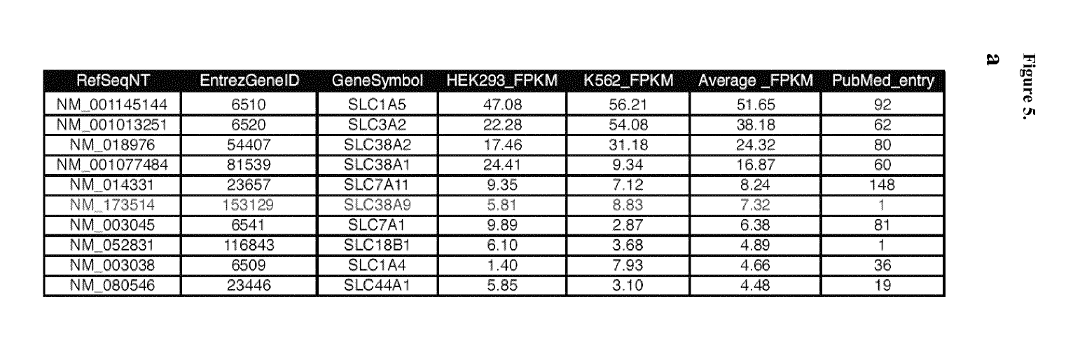

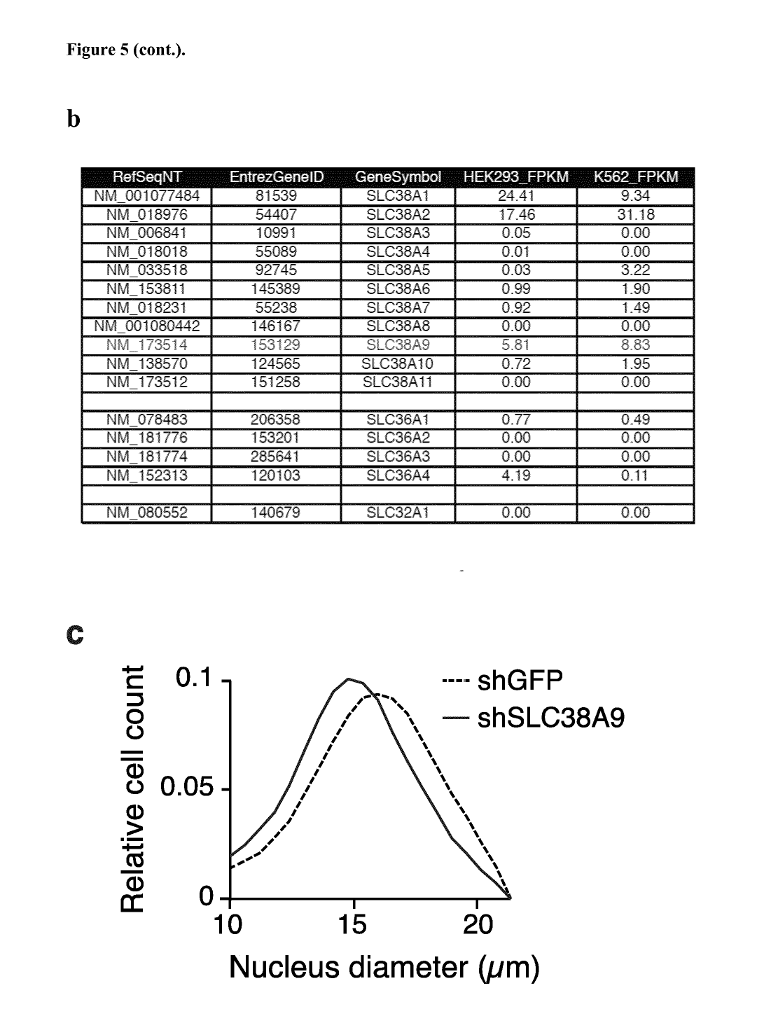

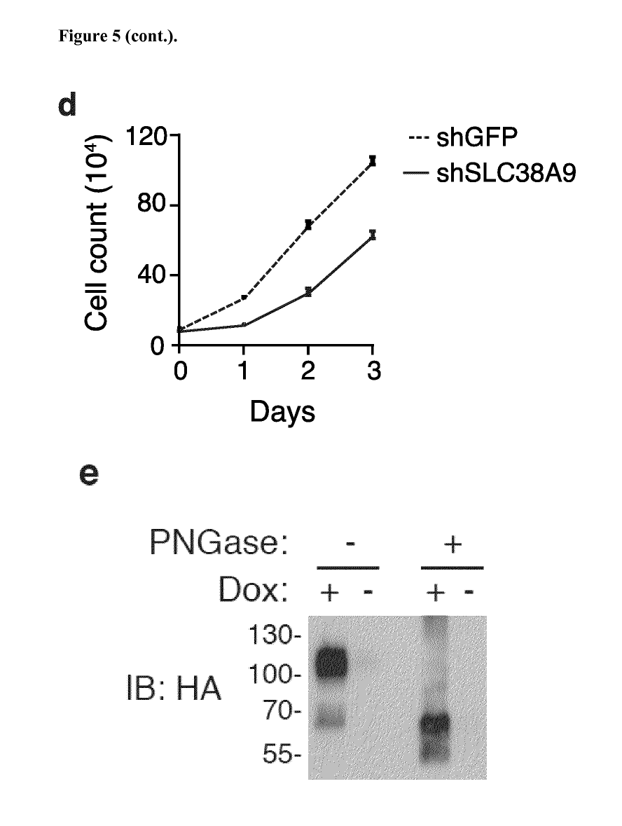

As a proof of principle, it was shown that suppression of SLC38A9 expression in HEK293T by shRNA resulted in a strong reduction of amino acid-induced mTORC1 activation (FIG. 4A). Cell size and cell proliferation was monitored after down-regulation of SLC38A9 by short hairpin RNA (shRNA) interference in HEK293T cells. Silencing of SLC38A9 resulted in a clear reduction of cell size and impairment in the ability of the targeted cells to proliferate, supporting a role of this protein in growth regulatory pathways (FIG. 5C-D).

Similar results were obtained when SLC38A9 expression was reduced by small interfering RNA (siRNA): silencing of SLC38A9 in HE293T and HeLa suppressed amino acid-induced mTORC1 activation with similar efficiency as silencing of the positive control Lamtor1 (FIG. 4C-D). Thus, loss of Sentor expression led to a reduction in cell size and proliferation and impaired mTORC1 activation by amino acids.

This shows that these and further antagonists described herein that inhibit the activity of SLC38A9 can be used to antagonize mTORC1 activation.

As mTORC1 is involved in cell growth²¹ and is hence a driving factor in the development and progress of diseases associated with mTORC1 activation (in particular aberrant mTORC1 activation), like proliferative diseases or metabolic disorders, the use of antagonists of SLC38A9 as shown and described herein, provides for a clear rationale for the use of such antagonists in the therapy of diseases associated with (aberrant) mTORC1 activation. Indeed, aberrant activation of mTORC1 in known in the art to be implicated in several disease, and in particular metabolic disorders like type 2 diabetes as well as in proliferative malignancies like cancer as described, inter alai, in Cornu (Current opinion in genetics & development 23, 53-62, doi:10.1016/j.gde.2012.12.005 (2013)), Laplante (Cell 149, 274-293, doi:10.1016/j.cell.2012.03.017 (2012)), Shaw (Nature 441, 424-430, doi:10.1038/nature04869 (2006)) and Efeyan (Trends Mol Med, 2012. 18(9): p. 524-33.)).

Thus, Sentor is a physical and functional component of the amino acid-sensing complex that controls the activation of mTOR. Given that amino acids are essential for mTORC1 activity, as growth factors cannot efficiently activate mTOR in their absence^{4,9}, targeting Sentor affects a crucial mechanism required for mTORC1 activation. Therefore, antagonists of Sentor can credibly be used in the treatment of diseases associated with (aberrant) mTORC1 activation.

The following relates to “SLC38A9” (“Sentor”), whose capacity as lysosomal amino acid sensor required for activation of mTOR/mTORC1 is shown herein.

As demonstrated in the herein provided experiments, SLC38A9 displays the characteristics expected for the lysosomal amino acid sensor required for activation of mTOR. Therefore, SLC38A9 is termed herein “Sentor” (“sensor of mTOR”). The terms “SLC38A9” and “Sentor” are used interchangeably herein.

The term “SLC38A9” as used herein refers to member 9 of the SLC38 family. The SLC38 (also known as sodium-coupled neutral amino acid transporter, SNAT) family counts eleven members, and is part of a major phylogenetic cluster of amino acid transporters comprising also the SLC32 and SLC36 families.

Four isoforms of “SLC38A9” are known. Corresponding nucleic acid sequences and amino acid sequences of isoform 1, 2, 3 and 4 are shown in SEQ ID NOs. 1 to 4 (isoform 1), 12-14 (isoform 2), 15 to 17 (isoform 3) and 18-20 (isoform 4), respectively.

Such nucleic acid sequences can be retrieved in public databases like NCBI using the following accession numbers:

### NCBI:

### Isoform1: NM_173514.3

### Isoform2: NM_001258286.1

### Isoform3: NM_001258287.1

### Isoform4: NM_001282429.1

Corresponding amino acid sequences can be retrieved in public database like NCBI using the following accession numbers:

### NCBI:

### Isoform1: NP_775785.2

### Isoform2: NP_001245215.1

### Isoform3: NP_001245216.1

### Isoform4: NP_001269358.1

Herein preferred is the use of isoform 1 of SLC38A9. An exemplary amino acid sequence of said isoform 1 is shown in SEQ ID NO. 3. Corresponding exemplary nucleic acid sequences are shown in SEQ ID NO: 1, 2 or 4. SEQ ID 1 or 2 show DNA sequences; whereas SEQ ID NO. 4 shows an mRNA sequence. The use of SLC38A9 being encoded by these nucleic acid sequences (or having this amino acid sequence) and being encoded by or having related sequences is, accordingly, preferred herein.

The term “SLC38A9” as used herein refers primarily to a protein. It is believed that “SLC38A9” can act as monomer or dimer. Thus, the term “SLC38A9” can refer to a monomer or dimer of a SLC38A9 protein as defined herein, in particular a monomer or dimer of isoform 1 of SLC38A9.

Accordingly, the SLC38A9 to be used herein can be selected from the group consisting of

(a) a polypeptide comprising an amino acid encoded by a nucleic acid molecule having the nucleic acid sequence as depicted in SEQ ID NO: 1, 2, 4, 12, 13, 15, 16, 18 and 19;
(b) a polypeptide having an amino acid sequence as depicted in SEQ ID NO:3, 14, 17 and 20;
(c) a polypeptide encoded by a nucleic acid molecule encoding a peptide having an amino acid sequence as depicted in SEQ ID NO: 3, 14, 17 and 20;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

In a preferred embodiment, the SLC38A9 to be used herein is selected from the group consisting of

(a) a polypeptide comprising an amino acid encoded by a nucleic acid molecule having the nucleic acid sequence as depicted in SEQ ID NO: 1, 2 or 4;
(b) a polypeptide having an amino acid sequence as depicted in SEQ ID NO:3;
(c) a polypeptide encoded by a nucleic acid molecule encoding a peptide having an amino acid sequence as depicted in SEQ ID NO: 3;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90% identity, particularly preferably at least 99% identity, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

In a particularly preferred embodiment, the SLC38A9 to be used herein is selected from the group consisting of

(a) a polypeptide comprising an amino acid encoded by a nucleic acid molecule having the nucleic acid sequence as depicted in SEQ ID NO: 1, 2 or 4;
(b) a polypeptide having an amino acid sequence as depicted in SEQ ID NO:3;
(c) a polypeptide having at least 99% identity to the polypeptide of any one of (a) or (b).

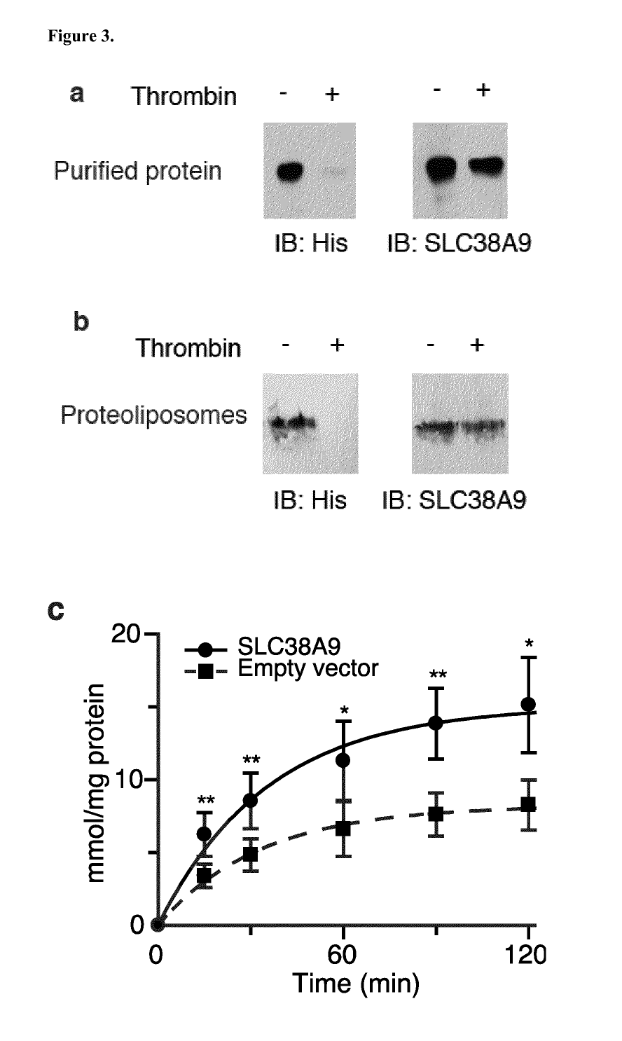

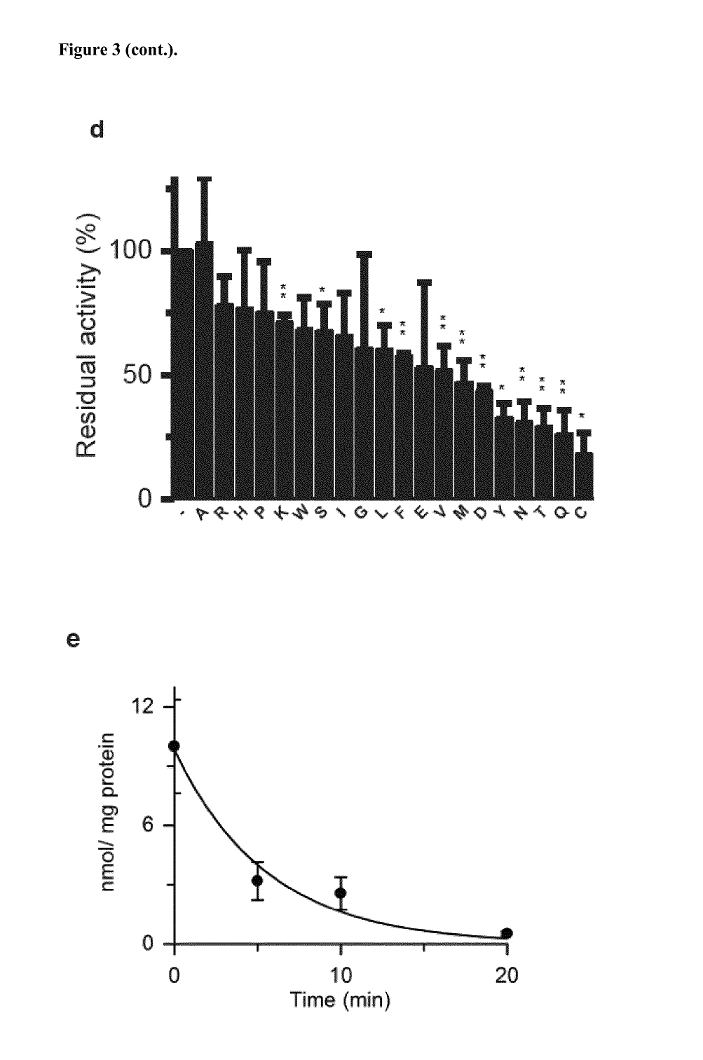

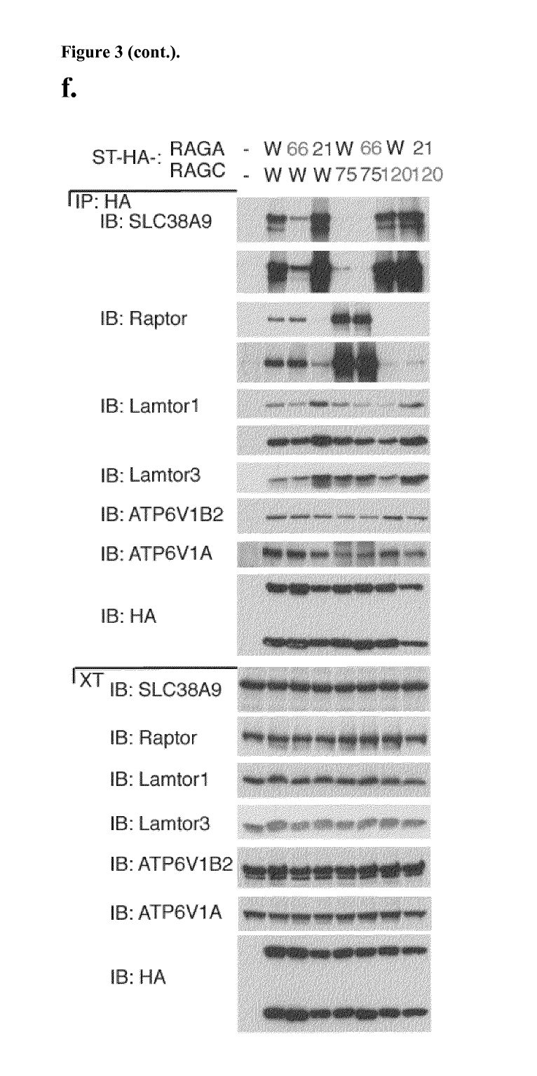

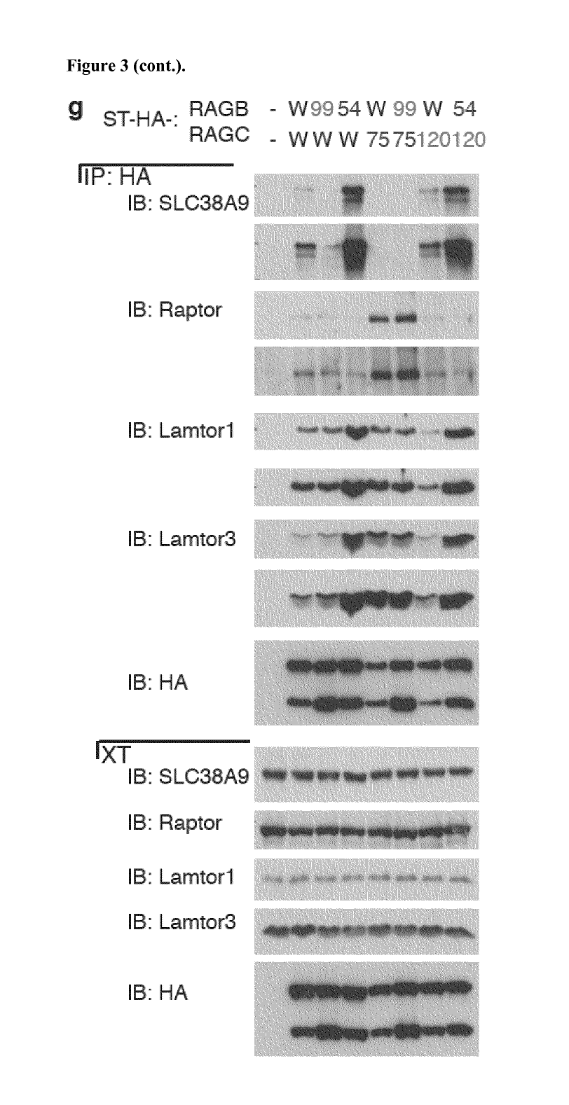

SLC38A9 to be used herein, in particular isoform 1 of SLC38A9 as defined herein, has the capacity/activity to transport amino acids and/or to bind to amino acids, particularly glutamine and cysteine; see FIGS. 3c-d and 10c. Also related amino acids can be transported and/or bound, like selenocysteine. The other amino acids shown in FIG. 3d can be bound and/or transported. FIG. 3d exemplifies the binding preference of SLC38A9 for amino acids (so that SLC38A9 has the highest binding capacity for glutamine and cysteine and the lowest binding capacity for alanine). The binding preference reflects the transport preference (so that SLC38A9 has the highest transport capacity for glutamine and cysteine and lowest transport capacity for alanine). SLC38A9 to be used herein has primarily the capacity/activity to transport amino acids, particularly glutamine, cysteine (or selenocysteine), or other amino acids. By transporting and/or binding to amino acids, particularly glutamine and cysteine (or selenocysteine), or other amino acids, SLC38A9 acts as sensor of the amino acid status and hence as activator of mTORC1.

It is demonstrated in the experiments provided herein that the transmembrane region of the SLC38A9 protein is primarily important for the activity of SLC38A9 to transport and/or bind to amino acids, in particular glutamine and cysteine.

The capacity/activity to transport amino acids and/or to bind to amino acids, particularly glutamine, resides in the eleven transmembrane-containing regions (113-561) of isoform 1 of SLC38A9. Accordingly, antagonists of SLC38A9 to be used herein interfere, in particular, inhibit/antagonize in particular this transport and/or binding activity/capacity. Therefore, the transmembrane region is the primary target of antagonists of SLC38A9 to be used and/or identified in accordance with the present invention.

In the following sequences of transmembrane-containing regions of SLC38A9 to be used as targets of antagonists of SLC38A9 are disclosed:

Isoform 1; amino acid sequence:
(SEQ ID NO: 68)
GYGKNTSLVTIFMIWNTMMGTSILSIPWGIKQAGFTTGMCVIILMGLLTL
YCCYRVVKSRTMMFSLDTTSWEYPDVCRHYFGSFGQWSSLLFSLVSLIGA
MIVYWVLMSNFLFNTGKFIFNFIHHINDTDTILSTNNSNPVICPSAGSGG
HPDNSSMIFYANDTGAQQFEKKWWDKSRTVPFYLVGLLLPLLNFKSPSFF
SKFNILGTVSVLYLIFLVTFKAVRLGFHLEFHWFIPTEFFVPEIRFQFPQ
LTGVLTLAFFIHNCIITLLKNNKKQENNVRDLCIAYMLVTLTYLYIGVLV
FASFPSPPLSKDCIEQNFLDNFPSSDTLSFIARIFLLFQMMTVYPLLGYL
ARVQLLGHIFGDIYPSIFHVLILNLIIVGAGVIMACFYPNIGGIIRYSGA
ACGLAFVFIYPSLIYIISLHQEERLTWPKLIFHVFIIILGVANLIVQFFM
Isoform 1, nucleotide sequence:
(SEQ ID NO: 67)
GGATACGGTAAAAACACCAGTTTAGTAACCATTTTTATGATTTGGAATAC
CATGATGGGAACATCTATACTAAGCATTCCTTGGGGCATAAAACAGGCTG
GATTTACTACTGGAATGTGTGTCATCATACTGATGGGCCTTTTAACACTT
TATTGCTGCTACAGAGTAGTGAAATCACGGACTATGATGTTTTCGTTGGA
TACCACTAGCTGGGAATATCCAGATGTCTGCAGACATTATTTCGGCTCCT
TTGGGCAGTGGTCGAGTCTCCTTTTCTCCTTGGTGTCTCTCATTGGAGCA
ATGATAGTTTATTGGGTGCTTATGTCAAATTTTCTTTTTAATACTGGAAA
GTTTATTTTTAATTTTATTCATCACATTAATGACACAGACACTATACTGA
GTACCAATAATAGCAACCCTGTGATTTGTCCAAGTGCCGGGAGTGGAGGC
CATCCTGACAACAGCTCTATGATTTTCTATGCCAATGACACAGGAGCCCA
ACAGTTTGAAAAGTGGTGGGATAAGTCCAGGACAGTCCCCTTTTATCTTG
TAGGGCTCCTCCTCCCACTGCTCAATTTCAAGTCTCCTTCATTTTTTTCA
AAATTTAATATCCTAGGCACAGTGTCTGTCCTTTATTTGATTTTCCTTGT
CACCTTTAAGGCTGTTCGCTTGGGATTTCATTTGGAATTTCATTGGTTTA
TACCAACAGAATTTTTTGTACCAGAGATAAGATTTCAGTTTCCACAGCTG
ACTGGAGTGCTTACCCTTGCTTTTTTTATTCATAATTGTATCATCACACT
CTTGAAGAACAACAAGAAACAAGAAAACAATGTGAGGGACTTGTGCATTG
CTTATATGCTGGTGACATTAACTTATCTCTATATTGGAGTCCTGGTTTTT
GCTTCATTTCCTTCACCACCATTATCCAAAGATTGTATTGAGCAGAATTT
TTTAGACAACTTCCCTAGCAGTGACACCCTGTCCTTCATTGCAAGGATAT
TCCTGCTGTTCCAGATGATGACTGTATACCCACTCTTAGGCTACCTGGCT
CGTGTCCAGCTTTTGGGCCATATCTTCGGTGACATTTATCCTAGCATTTT
CCATGTGCTGATTCTTAATCTAATTATTGTGGGAGCTGGAGTGATCATGG
CCTGTTTCTACCCAAACATAGGAGGGATCATAAGATATTCAGGAGCAGCA
TGTGGACTGGCCTTTGTATTCATATACCCATCTCTCATCTATATAATTTC
CCTCCACCAAGAAGAGCGTCTGACATGGCCTAAATTAATCTTCCACGTTT
TCATCATCATTTTGGGCGTGGCTAACCTGATTGTTCAGTTTTTTATGTGA
Isoform2, amino acid sequence:
(SEQ ID NO: 70)
GYGKNTSLVTIFMIWNTMMGTSILSIPWGIKQAGFTTGMCVIILMGLLTL
YCCYRVVKSRTMMFSLDTTSWEYPDVCRHYFGSFGQWSSLLFSLVSLIGA
MIVYWVLMSNFLFNTGKFIFNFIHHINDTDTILSTNNSNPVICPSAGSGG
HPDNSSMIFYANDTGAQQFEKWWDKSRTVPFYLVGLLLPLLNFKSPSFFS
KFNILGTVSVLYLIFLVTFKAVRLGFHLEFHWFIPTEFFVPEIRFQFPQL
TGVLTLAFFIHNCIITLLKNNKKQENNVRDLCIAYMLVTLTYLYIGVLVF
ASFPSPPLSKDCIEQNFLDNFPSSDTLSFIARIFLLFQMMTVYPLLGYLA
RVQLLGHIFGDIYPSIFHVLILNLIIVGAGVIMACFYPNIGGIIRYSGAA
CGLAFVFIYPSLIYIISLHQEERLTWPKLIFHVFIIILGVANLIVQFFM
Isoform2, nucleotide sequence:
(SEQ ID NO: 69)
GGATACGGTAAAAACACCAGTTTAGTAACCATTTTTATGATTTGGAATA
CCATGATGGGAACATCTATACTAAGCATTCCTTGGGGCATAAAACAGGC
TGGATTTACTACTGGAATGTGTGTCATCATACTGATGGGCCTTTTAACA
CTTTATTGCTGCTACAGAGTAGTGAAATCACGGACTATGATGTTTTCGT
TGGATACCACTAGCTGGGAATATCCAGATGTCTGCAGACATTATTTCGG
CTCCTTTGGGCAGTGGTCGAGTCTCCTTTTCTCCTTGGTGTCTCTCATT
GGAGCAATGATAGTTTATTGGGTGCTTATGTCAAATTTTCTTTTTAATA
CTGGAAAGTTTATTTTTAATTTTATTCATCACATTAATGACACAGACAC
TATACTGAGTACCAATAATAGCAACCCTGTGATTTGTCCAAGTGCCGGG
AGTGGAGGCCATCCTGACAACAGCTCTATGATTTTCTATGCCAATGACA
CAGGAGCCCAACAGTTTGAAAAGTGGTGGGATAAGTCCAGGACAGTCCC
CTTTTATCTTGTAGGGCTCCTCCTCCCACTGCTCAATTTCAAGTCTCCT
TCATTTTTTTCAAAATTTAATATCCTAGGCACAGTGTCTGTCCTTTATT
TGATTTTCCTTGTCACCTTTAAGGCTGTTCGCTTGGGATTTCATTTGGA
ATTTCATTGGTTTATACCAACAGAATTTTTTGTACCAGAGATAAGATTT
CAGTTTCCACAGCTGACTGGAGTGCTTACCCTTGCTTTTTTTATTCATA
ATTGTATCATCACACTCTTGAAGAACAACAAGAAACAAGAAAACAATGT
GAGGGACTTGTGCATTGCTTATATGCTGGTGACATTAACTTATCTCTAT
ATTGGAGTCCTGGTTTTTGCTTCATTTCCTTCACCACCATTATCCAAAG
ATTGTATTGAGCAGAATTTTTTAGACAACTTCCCTAGCAGTGACACCCT
GTCCTTCATTGCAAGGATATTCCTGCTGTTCCAGATGATGACTGTATAC
CCACTCTTAGGCTACCTGGCTCGTGTCCAGCTTTTGGGCCATATCTTCG
GTGACATTTATCCTAGCATTTTCCATGTGCTGATTCTTAATCTAATTAT
TGTGGGAGCTGGAGTGATCATGGCCTGTTTCTACCCAAACATAGGAGGG
ATCATAAGATATTCAGGAGCAGCATGTGGACTGGCCTTTGTATTCATAT
ACCCATCTCTCATCTATATAATTTCCCTCCACCAAGAAGAGCGTCTGAC
ATGGCCTAAATTAATCTTCCACGTTTTCATCATCATTTTGGGCGTGGCT
AACCTGATTGTTCAGTTTTTTATGTGA
Isoform3, amino acid sequence:
(SEQ ID NO: 72)
EGYGKNTSLVTIFMIWNTMMGTSILSIPWGIKQAGFTTGMCVIILMGLL
TLYCCYRVVKSRTMMFSLDTTSWEYPDVCRHYFGSFGQWSSLLFSLVSL
IGAMIVYWVLMSNFLFNTGKFIFNFIHHINDTDTILSTNNSNPVICPSA
GSGGHPDNSSMIFYANDTGAQQFEKWWDKSRTVPFYLVGLLLPLLNFKS
PSFFSKFNILEIRFQFPQLTGVLTLAFFIHNCIITLLKNNKKQENNVRD
LCIAYMLVTLTYLYIGVLVFASFPSPPLSKDCIEQNFLDNFPSSDTLSF
IARIFLLFQMMTVYPLLGYLARVQLLGHIFGDIYPSIFHVLILNLIIVG
AGVIMACFYPNIGGIIRYSGAACGLAFVFIYPSLIYIISLHQEERLTWP
KLIFHVFIIILGVANLIVQFFM
Isoform3, nucleotide sequence:
(SEQ ID NO: 71)
GAAGGATACGGTAAAAACACCAGTTTAGTAACCATTTTTATGATTTGGA
ATACCATGATGGGAACATCTATACTAAGCATTCCTTGGGGCATAAAACA
GGCTGGATTTACTACTGGAATGTGTGTCATCATACTGATGGGCCTTTTA
ACACTTTATTGCTGCTACAGAGTAGTGAAATCACGGACTATGATGTTTT
CGTTGGATACCACTAGCTGGGAATATCCAGATGTCTGCAGACATTATTT
CGGCTCCTTTGGGCAGTGGTCGAGTCTCCTTTTCTCCTTGGTGTCTCTC
ATTGGAGCAATGATAGTTTATTGGGTGCTTATGTCAAATTTTCTTTTTA
ATACTGGAAAGTTTATTTTTAATTTTATTCATCACATTAATGACACAGA
CACTATACTGAGTACCAATAATAGCAACCCTGTGATTTGTCCAAGTGCC
GGGAGTGGAGGCCATCCTGACAACAGCTCTATGATTTTCTATGCCAATG
ACACAGGAGCCCAACAGTTTGAAAAGTGGTGGGATAAGTCCAGGACAGT
CCCCTTTTATCTTGTAGGGCTCCTCCTCCCACTGCTCAATTTCAAGTCT
CCTTCATTTTTTTCAAAATTTAATATCCTAGAGATAAGATTTCAGTTTC
CACAGCTGACTGGAGTGCTTACCCTTGCTTTTTTTATTCATAATTGTAT
CATCACACTCTTGAAGAACAACAAGAAACAAGAAAACAATGTGAGGGAC
TTGTGCATTGCTTATATGCTGGTGACATTAACTTATCTCTATATTGGAG
TCCTGGTTTTTGCTTCATTTCCTTCACCACCATTATCCAAAGATTGTAT
TGAGCAGAATTTTTTAGACAACTTCCCTAGCAGTGACACCCTGTCCTTC
ATTGCAAGGATATTCCTGCTGTTCCAGATGATGACTGTATACCCACTCT
TAGGCTACCTGGCTCGTGTCCAGCTTTTGGGCCATATCTTCGGTGACAT
TTATCCTAGCATTTTCCATGTGCTGATTCTTAATCTAATTATTGTGGGA
GCTGGAGTGATCATGGCCTGTTTCTACCCAAACATAGGAGGGATCATAA
GATATTCAGGAGCAGCATGTGGACTGGCCTTTGTATTCATATACCCATC
TCTCATCTATATAATTTCCCTCCACCAAGAAGAGCGTCTGACATGGCCT
AAATTAATCTTCCACGTTTTCATCATCATTTTGGGCGTGGCTAACCTGA
TTGTTCAGTTTTTTATGTGA
Isoform 4, amino acid sequence:
(SEQ ID NO: 74)
MIWNTMMGTSILSIPWGIKQAGFTTGMCVIILMGLLTLYCCYRVVKSRT
MMFSLDTTSWEYPDVCRHYFGSFGQWSSLLFSLVSLIGAMIVYWVLMSN
FLFNTGKFIFNFIHHINDTDTILSTNNSNPVICPSAGSGGHPDNSSMIF
YANDTGAQQFEKWWDKSRTVPFYLVGLLLPLLNFKSPSFPSKFNILGTV
SVLYLIFLVTFKAVRLGFHLEFHWFIPTEFFVPEIRFQFPQLTGVLTLA
FFIHNCIITLLKNNKKQENNVRDLCIAYMLVTLTYLYIGVLVFASFPSP
PLSKDCIEQNFLDNFPSSDTLSFIARIFLLFQMMTVYPLLGYLARVQLL
GHIFGDIYPSIFHVLILNLIIVGAGVIMACFYPNIGGIIRYSGAACGLA
FVFIYPSLIYIISLHQEERLTWPKLIFHVFIIILGVANLIVQFFM
Isoform 4, nucleotide sequence
(SEQ ID NO: 73)
ATGATTTGGAATACCATGATGGGAACATCTATACTAAGCATTCCTTGGG
GCATAAAACAGGCTGGATTTACTACTGGAATGTGTGTCATCATACTGAT
GGGCCTTTTAACACTTTATTGCTGCTACAGAGTAGTGAAATCACGGACT
ATGATGTTTTCGTTGGATACCACTAGCTGGGAATATCCAGATGTCTGCA
GACATTATTTCGGCTCCTTTGGGCAGTGGTCGAGTCTCCTTTTCTCCTT
GGTGTCTCTCATTGGAGCAATGATAGTTTATTGGGTGCTTATGTCAAAT
TTTCTTTTTAATACTGGAAAGTTTATTTTTAATTTTATTCATCACATTA
ATGACACAGACACTATACTGAGTACCAATAATAGCAACCCTGTGATTTG
TCCAAGTGCCGGGAGTGGAGGCCATCCTGACAACAGCTCTATGATTTTC
TATGCCAATGACACAGGAGCCCAACAGTTTGAAAAGTGGTGGGATAAGT
CCAGGACAGTCCCCTTTTATCTTGTAGGGCTCCTCCTCCCACTGCTCAA
TTTCAAGTCTCCTTCATTTTTTTCAAAATTTAATATCCTAGGCACAGTG
TCTGTCCTTTATTTGATTTTCCTTGTCACCTTTAAGGCTGTTCGCTTGG
GATTTCATTTGGAATTTCATTGGTTTATACCAACAGAATTTTTTGTACC
AGAGATAAGATTTCAGTTTCCACAGCTGACTGGAGTGCTTACCCTTGCT
TTTTTTATTCATAATTGTATCATCACACTCTTGAAGAACAACAAGAAAC
AAGAAAACAATGTGAGGGACTTGTGCATTGCTTATATGCTGGTGACATT
AACTTATCTCTATATTGGAGTCCTGGTTTTTGCTTCATTTCCTTCACCA
CCATTATCCAAAGATTGTATTGAGCAGAATTTTTTAGACAACTTCCCTA
GCAGTGACACCCTGTCCTTCATTGCAAGGATATTCCTGCTGTTCCAGAT
GATGACTGTATACCCACTCTTAGGCTACCTGGCTCGTGTCCAGCTTTTG
GGCCATATCTTCGGTGACATTTATCCTAGCATTTTCCATGTGCTGATTC
TTAATCTAATTATTGTGGGAGCTGGAGTGATCATGGCCTGTTTCTACCC
AAACATAGGAGGGATCATAAGATATTCAGGAGCAGCATGTGGACTGGCC
TTTGTATTCATATACCCATCTCTCATCTATATAATTTCCCTCCACCAAG
AAGAGCGTCTGACATGGCCTAAATTAATCTTCCACGTTTTCATCATCAT
TTTGGGCGTGGCTAACCTGATTGTTCAGTTTTTTATGTGA

Accordingly, the “transmembrane (-containing) regions of SLC38A9” to be used herein can be selected from the group consisting of

(a) a polypeptide comprising an amino acid encoded by a nucleic acid molecule having the nucleic acid sequence as depicted in SEQ ID NO: 67, 69, 71 or 73;
(b) a polypeptide having an amino acid sequence as depicted in SEQ ID NO: 68, 70, 72 or 74;
(c) a polypeptide encoded by a nucleic acid molecule encoding a peptide having an amino acid sequence as depicted in SEQ ID NO: 67, 69, 71 or 73;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90%, particularly preferably at least 99%, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

In a particularly preferred embodiment, the “transmembrane (-containing) region of SLC38A9” to be used herein is selected from the group consisting of

(a) a polypeptide comprising an amino acid encoded by a nucleic acid molecule having the nucleic acid sequence as depicted in SEQ ID NO: 67;
(b) a polypeptide having an amino acid sequence as depicted in SEQ ID NO: 68;
(c) a polypeptide encoded by a nucleic acid molecule encoding a peptide having an amino acid sequence as depicted in SEQ ID NO: 67;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90%, particularly preferably at least 99%, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

It is demonstrated in the experiments provided herein that the cytoplasmic region of the SLC38A9 protein is important for interaction with the Ragulator/RAG GTPases complex, for example for interaction with LAMTOR1, LAMTOR3, RAGA and RAGC proteins.

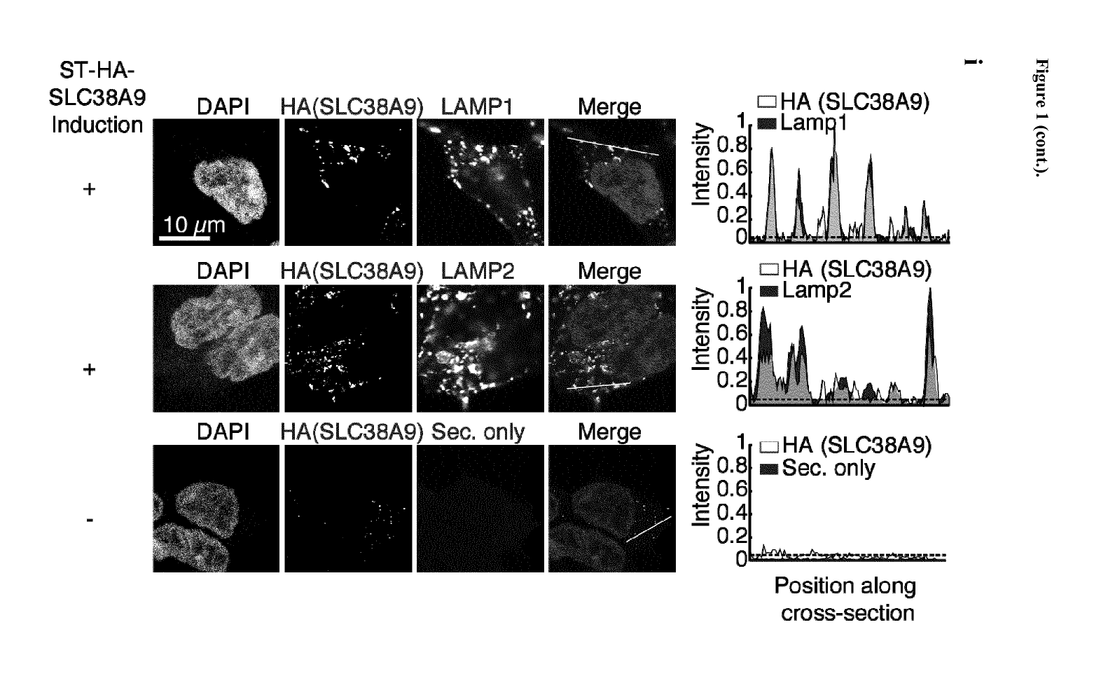

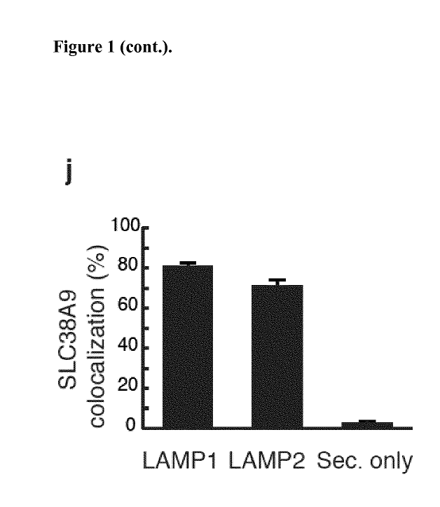

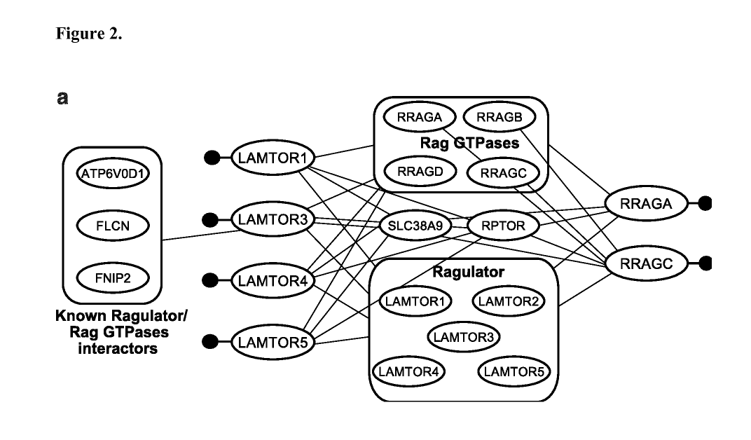

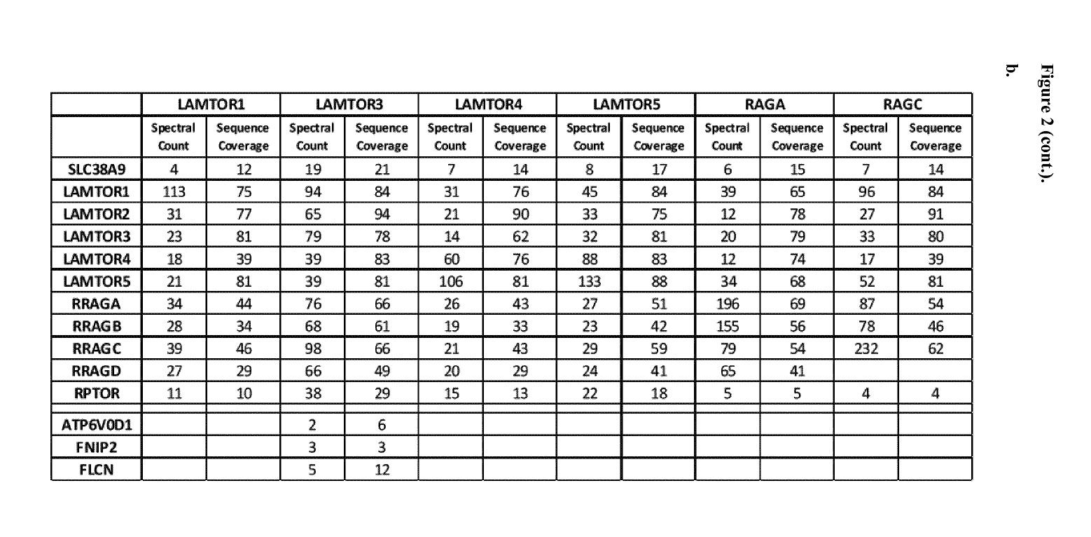

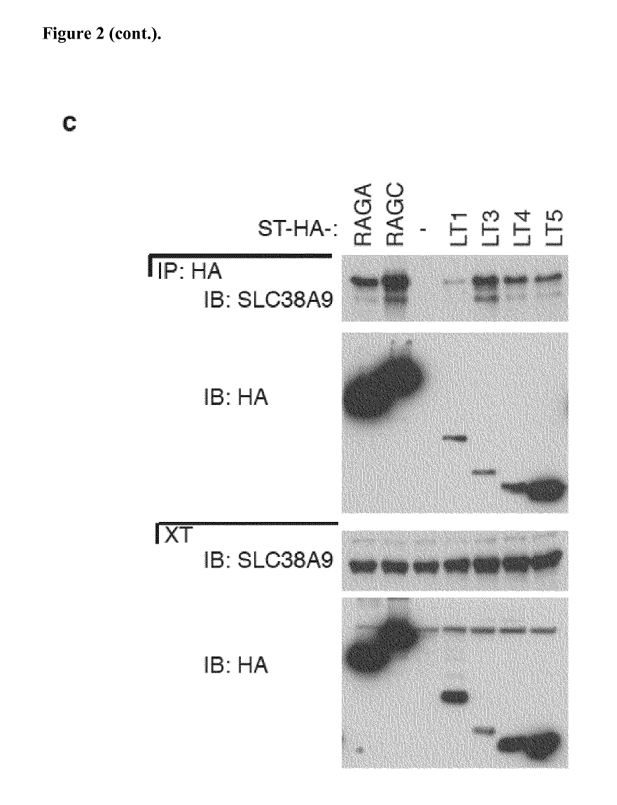

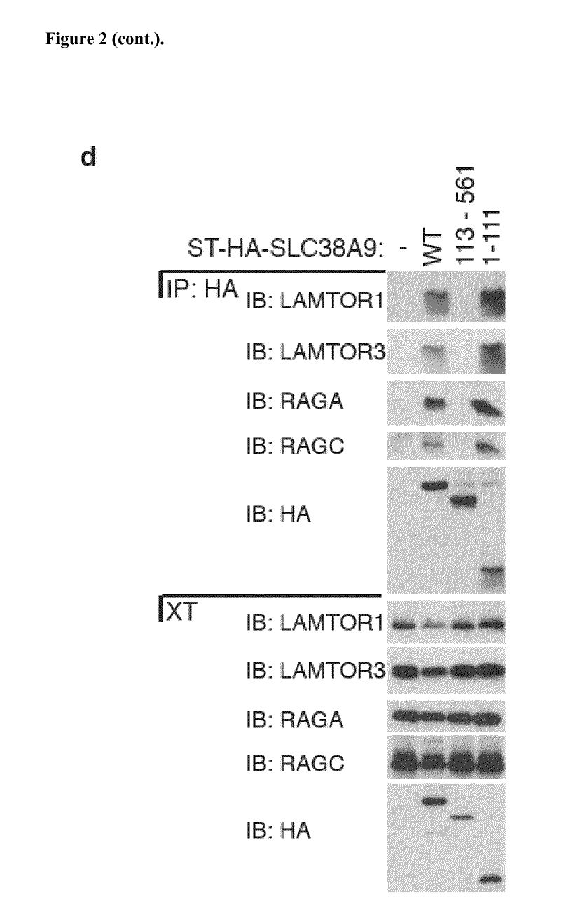

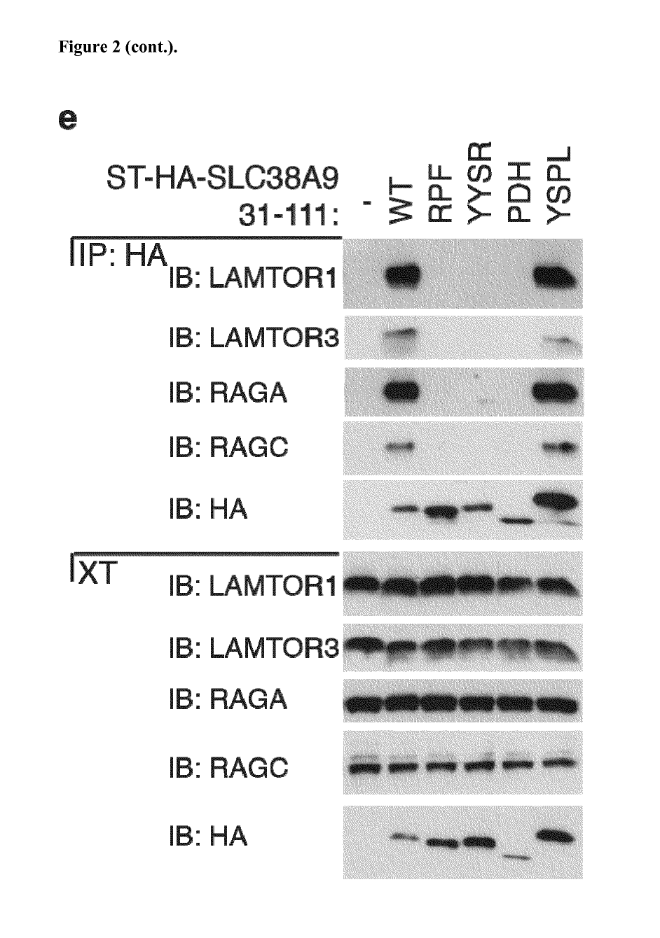

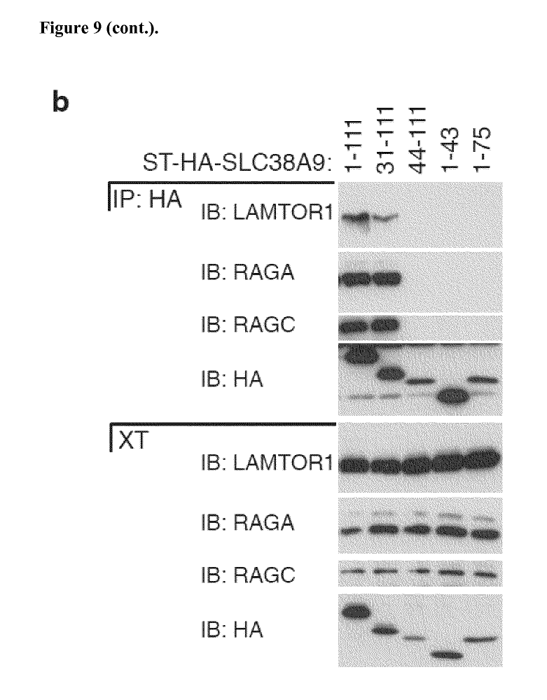

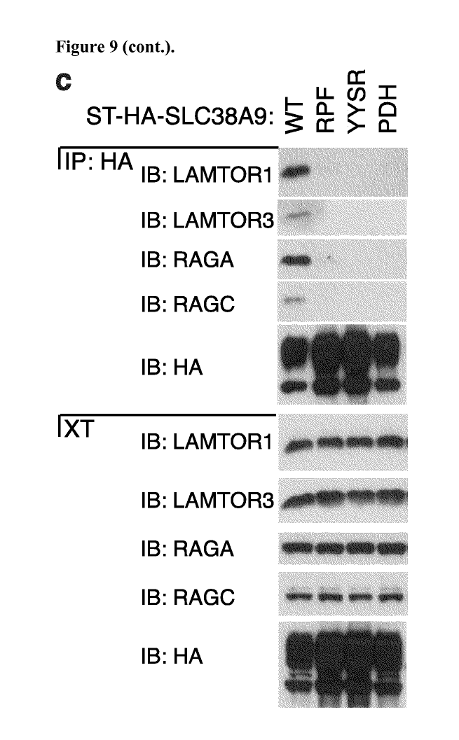

As shown in the experimental part, SLC38A9 (isoform 1) deletion constructs encoding the N-terminal cytoplasmic tail (amino acids 1-111) of isoform 1 of SLC38A9 or the remaining eleven transmembrane-containing regions (113-561) of isoform 1 of SLC38A9 were generated. In a more refined analysis, SLC38A9 (isoform 1) deletion constructs encoding the N-terminal cytoplasmic tail (amino acids 1-112) of isoform 1 of SLC38A9 were generated. It was demonstrated that the cytoplasmic region of SLC38A9 (isoform 1) retained the ability to interact with endogenous LAMTOR1, LAMTOR3, RAGA and RAGC proteins similar to the full-length protein, whereas binding was completely lost when the region was deleted (FIG. 2D). This indicated that the cytoplasmic tail, devoid of any transmembrane region, is required and sufficient to bind the Ragulator/RAG GTPases complex. Further deletion studies mapped the minimal binding region to amino acids 31-111 of isoform 1 of SLC38A9 (FIGS. 9A and B). More refined deletion studies mapped the binding region to amino acids 31-112 of isoform 1 of SLC38A9 (FIG. 9A). Further, four conserved motifs in the region (38RPF40, 70YYSR73, 85PDH87 and 98YSPL101) were identified and individually mutated to alanine (FIG. 9A). Disruption of any of the first three motifs completely abolished the binding ability of the cytoplasmic region of SLC38A9 (isoform 1) towards LAMTOR1, LAMTOR3, RAGA and RAGC whereas mutation of the fourth motif had no effect (FIG. 2E). This observation was also confirmed in the context of full length SLC38A9 (isoform 1) (FIG. 9C). These results defined the unique cytoplasmic region of SLC38A9 as responsible for the interaction with the lysosomal mTOR-activating machinery and indicated that evolutionary conserved motifs are required for this interaction to occur.

SLC38A9 to be used herein, in particular isoform 1 of SLC38A9 as defined herein, has, accordingly, the capacity/activity to interact with the Ragulator/RAG GTPases complex, for example the capacity/activity to interact with LAMTOR1, LAMTOR3, RAGA and/or RAGC protein(s). Transport of amino acids by SLC38A9 can influence the interaction with/binding to the Ragulator/RAG GTPases complex and/or modify the activity of RAG GTPases.

Therefore, the cytoplasmic region of SLC38A9 (protein) is a target region for the antagonists of SLC38A9 to be used herein. For example, antagonists of SLC38A9 may interfere with or inhibit interaction of SLC38A9 with binding partners, in particular with the Ragulator/RAG GTPases complex as defined herein, and thereby antagonize SLC38A9 (activity). Thus, herein preferred are antagonists of SLC38A9 that specifically target the cytoplasmic region of SLC38A9 (protein). For example, small molecule drugs or binding molecules (like peptide aptamers or intramers) to be used as antagonists of SLC38A9 herein can antagonize SLC38A9 by specifically binding to the cytoplasmic region of the SLC38A9 protein (or parts thereof and/or conserved motifs thereof as defined further below). Aptamers, intramers (in particular oligonucleic acid aptamers/intramers), siRNA, shRNA, miRNA, dsRNA, stRNA, antisense molecules and the like to be used as antagonists of SLC38A9 herein can antagonize SLC38A9 by specifically binding to a nucleic acid molecule having a sequence encoding the cytoplasmic region of the SLC38A9 protein (or parts thereof and/or conserved motifs thereof as defined further below).

The term “cytoplasmic region of SLC38A9” (or, more precisely, “cytoplasmic region of the SLC38A9 protein”) as used herein refers primarily to the N-terminal amino acid sequence of SLC38A9, i.e. the amino acid sequence of the full-length SLC38A9 protein excluding the eleven transmembrane-containing regions. A preferred “cytoplasmic region of SLC38A9” to be used herein is the “cytoplasmic region of isoform 1 of SLC38A9”.

The N-terminal cytoplasmic region of SLC38A9 (isoform 1) ranges from amino acid positions 1 to 120 of the full length protein (e.g. as shown in SEQ ID NO. 3). The N-terminal cytoplasmic region of SLC38A9 (isoform 1) of from amino acid positions 1 to 111, or of from amino acid positions 1 to 112, of the full length protein (e.g. as shown in SEQ ID NO. 3) is the part that is shown herein to be required for the binding to the RAG/Ragulator complex. Other cytoplasmic parts of the SLC38A9 protein (in particular isoform 1), like the one between the transmembrane domains are not sufficient to bind RAG/ragulator as deletion of amino acids 1-112 abrogates binding. Yet, these cytoplasmic parts may also be a valuable target for antagonists to be used herein, e.g. in addition to the N-terminal cytoplasmic region of SLC38A9 as defined herein.

Preferably, the N-terminal cytoplasmic region corresponds to (i.e. consists of) amino acids 1 to 111, more preferably to amino acids 1 to 112, of the full length SLC38A9 protein. An exemplary sequence of a full length SLC38A9 protein of the herein preferred isoform 1 is shown in SEQ ID NO. 3. An accordingly preferred N-terminal cytoplasmic region of SLC38A9 protein is as follows:

(SEQ ID NO: 22)
MANMNSDSRHLGTSEVDHERDPGPMNIQFEPSDLRSKRPFCIEPTNIVNV
NHVIQRVSDHASAMNKRIHYYSRLTTPADKALIAPDHVVPAPEECYVYSP
LGSAYKLQSYT

A corresponding nucleic acid sequence is as follows:

(SEQ ID NO: 21)
ATGGCAAATATGAATAGTGATTCTAGGCATCTTGGCACCTCTGAGGTAGA
TCATGAAAGAGATCCTGGACCTATGAATATCCAGTTTGAGCCATCGGATC
TAAGATCCAAAAGGCCTTTCTGTATAGAGCCCACAAACATCGTGAATGTG
AATCATGTCATTCAGAGGGTTAGTGACCATGCCTCTGCCATGAACAAGAG
AATTCATTACTACAGCCGGCTCACCACTCCTGCAGACAAGGCACTGATTG
CCCCAGACCATGTAGTTCCAGCTCCAGAAGAGTGCTATGTGTATAGTCCA
TTGGGCTCTGCTTATAAACTTCAAAGTTACACT

A herein preferred N-terminal cytoplasmic region of SLC38A9 protein corresponding to (i.e. consisting of) amino acids 1 to 112 of the full length SLC38A9 protein is as follows:

(SEQ ID NO: 97)
MANMNSDSRHLGTSEVDHERDPGPMNIQFEPSDLRSKRPFCIEPTNIVNV
NHVIQRVSDHASAMNKRIHYYSRLTTPADKALIAPDHVVPAPEECYVYSP
LGSAYKLQSYTE

A corresponding nucleic acid sequence is as follows:

(SEQ ID NO: 96)
ATGGCAAATATGAATAGTGATTCTAGGCATCTTGGCACCTCTGAGGTAG
ATCATGAAAGAGATCCTGGACCTATGAATATCCAGTTTGAGCCATCGGA
TCTAAGATCCAAAAGGCCTTTCTGTATAGAGCCCACAAACATCGTGAAT
GTGAATCATGTCATTCAGAGGGTTAGTGACCATGCCTCTGCCATGAACA
AGAGAATTCATTACTACAGCCGGCTCACCACTCCTGCAGACAAGGCACT
GATTGCCCCAGACCATGTAGTTCCAGCTCCAGAAGAGTGCTATGTGTAT
AGTCCATTGGGCTCTGCTTATAAACTTCAAAGTTACACTGAA

Isoforms 2, 3 and 4 of SLC38A9 may, like isoform 1, have the capacity to interact with the RAG/Ragulator complex. In particular isoforms 2 and 3 of SLC38A9 may have the capacity to bind to the RAG/Ragulator complex. Alternatively isoforms 2, 3 and 4 of SLC38A9 may, like isoform 1, have the capacity to bind to/transport amino acids. It may also be that isoforms 2, 3 and 4 of SLC38A9 interact with isoform 1, potentially supporting its activity. For these reasons, antagonizing/antagonists of isoforms 2, 3 and 4 of SLC38A9 (as defined herein further below) in accordance with the present invention is envisaged herein, too.

Compared to isoform 1 of SLC38A9 (see, for example, the sequence shown in SEQ ID NO. 3), isoform2 lack amino acids 1 to 63, isoform 3 lacks amino acids 1 to 37 that are substituted with: MAICILTWRI and Isoform 4 lacks amino acids 1 to 124.

N-terminal cytoplasmic regions of isoforms 2 and 3 of the SLC38A9 protein that correspond to the N-terminal cytoplasmic region of isoform 1, in particular amino acids 1 to 111, and that may serve as targets for antagonists provided herein are as follows:

Isoform 2 (aa 1-48 corresponding to the 1-111
region of isoform 1):
(SEQ ID NO: 54)
MNKRIHYYSRLTTPADKALIAPDHVVPAPEECYVYSPLGSAYKLQSYT

The corresponding nucleic acid sequences is as follows:

(SEQ ID NO: 53)
ATGAACAAGAGAATTCATTACTACAGCCGGCTCACCACTCCTGCAGACAA
GGCACTGATTGCCCCAGACCATGTAGTTCCAGCTCCAGAAGAGTGCTATG
TGTATAGTCCATTGGGCTCTGCTTATAAACTTCAAAGTTACACT

Isoform 3 (aa 1-84 corresponding to the 1-111
region of isoform 1):
(SEQ ID NO: 56)
MAICILTWRIRPFCIEPTNIVNVNHVIQRVSDHASAMNKRIHYYSRLTTP
ADKALIAPDHVVPAPEECYVYSPLGSAYKLQSYT

The corresponding nucleic acid sequences is as follows:

(SEQ ID NO: 55)
ATGGCTATTTGCATTTTAACATGGAGAATCCGGCCTTTCTGTATAGAGCC
CACAAACATCGTGAATGTGAATCATGTCATTCAGAGGGTTAGTGACCATG
CCTCTGCCATGAACAAGAGAATTCATTACTACAGCCGGCTCACCACTCCT
GCAGACAAGGCACTGATTGCCCCAGACCATGTAGTTCCAGCTCCAGAAGA
GTGCTATGTGTATAGTCCATTGGGCTCTGCTTATAAACTTCAAAGTTACA
CT

N-terminal cytoplasmic regions of isoforms 2 and 3 of the SLC38A9 protein that correspond to the N-terminal cytoplasmic region of isoform 1, in particular amino acids 1 to 112, and that may serve as targets for antagonists provided herein are as follows:

Isoform 2 (aa 1-49 corresponding to the 1-112
region of isoform 1):
(SEQ ID NO: 99)
MNKRIHYYSRLTTPADKALIAPDHVVPAPEECYVYSPLGSAYKLQSYTE

The corresponding nucleic acid sequences is as follows:

(SEQ ID NO: 98)
ATGAACAAGAGAATTCATTACTACAGCCGGCTCACCACTCCTGCAGACAAG
GCACTGATTGCCCCAGACCATGTAGTTCCAGCTCCAGAAGAGTGCTATGTG
TATAGTCCATTGGGCTCTGCTTATAAACTTCAAAGTTACACTGAA

Isoform 3 (aa 1-85 corresponding to the 1-112
region of isoform 1):
(SEQ ID NO: 101)
MAICILTWRIRPFCIEPTNIVNVNHVIQRVSDHASAMNKRIHYYSRLTT
PADKALIAPDHVVPAPEECYVYSPLGSAYKLQSYTE

The corresponding nucleic acid sequences is as follows:

(SEQ ID NO: 100)
ATGGCTATTTGCATTTTAACATGGAGAATCCGGCCTTTCTGTATAGAGC
CCACAAACATCGTGAATGTGAATCATGTCATTCAGAGGGTTAGTGACCA
TGCCTCTGCCATGAACAAGAGAATTCATTACTACAGCCGGCTCACCACT
CCTGCAGACAAGGCACTGATTGCCCCAGACCATGTAGTTCCAGCTCCAG
AAGAGTGCTATGTGTATAGTCCATTGGGCTCTGCTTATAAACTTCAAAG
TTACACTGAA

Accordingly, the “cytoplasmic region of SLC38A9” to be used herein can be selected from the group consisting of

(a) a polypeptide comprising an amino acid encoded by a nucleic acid molecule having the nucleic acid sequence as depicted in SEQ ID NO: 21, 53, 55, 96, 98 or 100;
(b) a polypeptide having an amino acid sequence as depicted in SEQ ID NO: 22, 54, 56, 97, 99 or 101;
(c) a polypeptide encoded by a nucleic acid molecule encoding a peptide having an amino acid sequence as depicted in SEQ ID NO: 22, 54, 56, 97, 99 or 101;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90%, particularly preferably at least 99%, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

In a preferred embodiment, the “cytoplasmic region of SLC38A9” to be used herein is selected from the group consisting of

(a) a polypeptide comprising an amino acid encoded by a nucleic acid molecule having the nucleic acid sequence as depicted in SEQ ID NO: 21 or 96;
(b) a polypeptide having an amino acid sequence as depicted in SEQ ID NO: 22 or 97;
(c) a polypeptide encoded by a nucleic acid molecule encoding a peptide having an amino acid sequence as depicted in SEQ ID NO: 22 or 97;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90%, particularly preferably at least 99%, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

Further deletion studies mapped the minimal binding region to amino acids 31-111 of the isoform 1 of the SLC38A9 protein (FIGS. 9A and B). In refined deletion studies the binding region was mapped to amino acids 31-112 of the isoform 1 of the SLC38A9 protein (FIG. 9A). Therefore, the cytoplasmic region of SLC38A9 (protein) corresponding to amino acids 31-111 isoform 1 of the SLC38A9 protein, particularly the cytoplasmic region of SLC38A9 (protein) corresponding to amino acids 31-112 isoform 1 of the SLC38A9 protein is a particularly preferred target region for the antagonists of SLC38A9 to be used herein. Thus, herein preferred are antagonists of SLC38A9 that specifically target the amino acid sequence corresponding to positions 31-111 of isoform 1 of SLC38A9 (protein), particularly antagonists of SLC38A9 that specifically target the amino acid sequence corresponding to positions 31-112 of isoform 1 of SLC38A9.

In the following exemplary nucleotide and amino acid sequences corresponding to positions 31-111 of isoform 1 of SLC38A9 are shown:

Nucleotide sequence encoding amino acids 31-111
of isoform 1:
(SEQ ID NO: 57)
CCATCGGATCTAAGATCCAAAAGGCCTTTCTGTATAGAGCCCACAAACA
TCGTGAATGTGAATCATGTCATTCAGAGGGTTAGTGACCATGCCTCTGC
CATGAACAAGAGAATTCATTACTACAGCCGGCTCACCACTCCTGCAGAC
AAGGCACTGATTGCCCCAGACCATGTAGTTCCAGCTCCAGAAGAGTGCT
ATGTGTATAGTCCATTGGGCTCTGCTTATAAACTTCAAAGTTACACT
Amino acid sequence of amino acids 31-111 of
isoform 1:
(SEQ ID NO: 58)
PSDLRSKRPFCIEPTNIVNVNHVIQRVSDHASAMNKRIHYYSRLTTPAD
KALIAPDHVVPAPEECYVYSPLGSAYKLQSYT
Nucleotide sequence encoding amino acids 1 to 48
of isoform 2 (corresponding to amino acids 64-111
of isoform 1):
(SEQ ID NO: 59)
ATGAACAAGAGAATTCATTACTACAGCCGGCTCACCACTCCTGCAGACA
AGGCACTGATTGCCCCAGACCATGTAGTTCCAGCTCCAGAAGAGTGCTA
TGTGTATAGTCCATTGGGCTCTGCTTATAAACTTCAAAGTTACACT
Amino acid sequence of amino acids 1 to 48 of
isoform 2 (corresponding to the 64-111 region of
isoform 1):
(SEQ ID NO: 60)
MNKRIHYYSRLTTPADKALIAPDHVVPAPEECYVYSPLGSAYKLQSYT
Nucleotide sequence encoding amino acids 11 to 84
of isoform 3 (corresponding to amino acids 31-111
of isoform 1):
(SEQ ID NO: 61)
CGGCCTTTCTGTATAGAGCCCACAAACATCGTGAATGTGAATCATGTCA
TTCAGAGGGTTAGTGACCATGCCTCTGCCATGAACAAGAGAATTCATTA
CTACAGCCGGCTCACCACTCCTGCAGACAAGGCACTGATTGCCCCAGAC
CATGTAGTTCCAGCTCCAGAAGAGTGCTATGTGTATAGTCCATTGGGCT
CTGCTTATAAACTTCAAAGTTACACT
Amino acid sequence of amino acids 11 to 84 of
isoform 3 (corresponding to amino acids 31-111 of
isoform 1):
(SEQ ID NO: 62)
RPFCIEPTNIVNVNHVIQRVSDHASAMNKRIHYYSRLTTPADKALIAPD
HVVPAPEECYVYSPLGSAYKLQSYT

In the following exemplary nucleotide and amino acid sequences corresponding to positions 31-112 of isoform 1 of SLC38A9 are shown:

Nucleotide sequence encoding amino acids 31-112
of isoform 1:
(SEQ ID NO: 102)
CCATCGGATCTAAGATCCAAAAGGCCTTTCTGTATAGAGCCCACAAACA
TCGTGAATGTGAATCATGTCATTCAGAGGGTTAGTGACCATGCCTCTGC
CATGAACAAGAGAATTCATTACTACAGCCGGCTCACCACTCCTGCAGAC
AAGGCACTGATTGCCCCAGACCATGTAGTTCCAGCTCCAGAAGAGTGCT
ATGTGTATAGTCCATTGGGCTCTGCTTATAAACTTCAAAGTTACACTGA
A
Amino acid sequence of amino acids 31-112 of
isoform 1:
(SEQ ID NO: 103)
PSDLRSKRPFCIEPTNIVNVNHVIQRVSDHASAMNKRIHYYSRLTTPAD
KALIAPDHVVPAPEECYVYSPLGSAYKLQSYTE
Nucleotide sequence encoding amino acids 1 to 49
of isoform 2 (corresponding to amino acids 64-112
of isoform 1):
(SEQ ID NO: 104)
ATGAACAAGAGAATTCATTACTACAGCCGGCTCACCACTCCTGCAGACA
AGGCACTGATTGCCCCAGACCATGTAGTTCCAGCTCCAGAAGAGTGCTA
TGTGTATAGTCCATTGGGCTCTGCTTATAAACTTCAAAGTTACACTGAA
Amino acid sequence of amino acids 1 to 49 of
isoform 2 (corresponding to the 64-112 region of
isoform 1):
(SEQ ID NO: 105)
MNKRIHYYSRLTTPADKALIAPDHVVPAPEECYVYSPLGSAYKLQSYTE
Nucleotide sequence encoding amino acids 11 to 85
of isoform 3 (corresponding to amino acids 31-112
of isoform 1):
(SEQ ID NO: 106)
CGGCCTTTCTGTATAGAGCCCACAAACATCGTGAATGTGAATCATGTCA
TTCAGAGGGTTAGTGACCATGCCTCTGCCATGAACAAGAGAATTCATTA
CTACAGCCGGCTCACCACTCCTGCAGACAAGGCACTGATTGCCCCAGAC
CATGTAGTTCCAGCTCCAGAAGAGTGCTATGTGTATAGTCCATTGGGCT
CTGCTTATAAACTTCAAAGTTACACTGAA
Amino acid sequence of amino acids 11 to 85 of
isoform 3 (corresponding to amino acids 31-112 of
isoform 1):
(SEQ ID NO: 107)
RPFCIEPTNIVNVNHVIQRVSDHASAMNKRIHYYSRLTTPADKALIAPD
HVVPAPEECYVYSPLGSAYKLQSYTE

Accordingly, the “cytoplasmic region of SLC38A9” to be used herein can be selected from the group consisting of

(a) a polypeptide comprising an amino acid encoded by a nucleic acid molecule having the nucleic acid sequence as depicted in SEQ ID NO: 57, 59, 61, 102, 104, or 106;
(b) a polypeptide having an amino acid sequence as depicted in SEQ ID NO: 58, 60, 62, 103, 105, or 107;
(c) a polypeptide encoded by a nucleic acid molecule encoding a peptide having an amino acid sequence as depicted in SEQ ID NO: 58, 60 62, 103, 105, or 107;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90%, particularly preferably at least 99%, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

In a particularly preferred embodiment, the “cytoplasmic region of SLC38A9” to be used herein is selected from the group consisting of

(a) a polypeptide comprising an amino acid encoded by a nucleic acid molecule having the nucleic acid sequence as depicted in SEQ ID NO: 57 or 102;
(b) a polypeptide having an amino acid sequence as depicted in SEQ ID NO: 58 or 103;
(c) a polypeptide encoded by a nucleic acid molecule encoding a peptide having an amino acid sequence as depicted in SEQ ID NO: 58 or 103;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity, preferably at least 90%, particularly preferably at least 99%, to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

Further, disruption of three conserved motifs in the cytoplasmic region (38RPF40, 70YYSR73, 85PDH87) abolished the binding ability of the cytoplasmic region of SLC38A9 (isoform 1) towards LAMTOR1, LAMTOR3, RAGA and RAGC. Therefore, these conserved motifs of SLC38A9 (protein) corresponding to amino acid positions 38-40 of isoform 1 of the SLC38A9 protein, 70 to 73 of isoform 1 of the SLC38A9 protein and/or 85 to 87 of isoform 1 of the SLC38A9 protein are also (a) particularly preferred target region(s) for the antagonists of SLC38A9 to be used herein. Thus, herein preferred are antagonists of SLC38A9 that specifically target the amino acid sequences corresponding to amino acid positions 38-40 of isoform 1 of the SLC38A9 protein, 70 to 73 of isoform 1 of the SLC38A9 protein and/or 85 to 87 of isoform 1 of the SLC38A9 protein.

Corresponding conserved motifs in isoforms 2 and 3 that can be targeted by antagonists to be used herein are as follows:

### Isoform 2:

first motif not present
second motif: 7YYSR10 (SEQ ID NO: 75) (19TACTACAGCCGG30 (SEQ ID NO: 76)),
third motif: 22PDH24 (64CCAGACCAT72)

### Isoform3:

first motif: 11RPF13(31CGGCCTTTC39),
second motif: 43YYSR46 (SEQ ID NO: 75) (127TACTACAGCCGG138 (SEQ ID NO: 76)),
third motif: 58PDH60 (172CCAGACCAT180)

The following relates to diseases associated with mTORC1 activation, in particular aberrant mTORC1 activation.

As a proof of principle, it was shown in the appended examples that suppression of SLC38A9 expression in HEK293T by shRNA resulted in a strong reduction of amino acid-induced mTORC1 activation (FIG. 4A). Cell size and cell proliferation was monitored after down-regulation of SLC38A9 by short hairpin RNA (shRNA) interference in HEK293T cells. Silencing of SLC38A9 resulted in a clear reduction of cell size and impairment in the ability of the targeted cells to proliferate, supporting a role of this protein in growth regulatory pathways (FIG. 5C-D). Similar results were obtained when SLC38A9 expression was reduced by small interfering RNA (siRNA): silencing of SLC38A9 suppressed amino acid-induced mTORC1 activation with similar efficiency as silencing of the positive control Lamtor1 (FIG. 4C). The shRNA to be used herein can target the 5′ UTR. The siRNA to be used herein can target regions that are encoding for the cytoplasmic region.

As mTORC1 is involved in cell growth²¹ and is hence, a driving factor in the development and progress of diseases associated with mTORC1 activation, like proliferative diseases or metabolic disorders, the use of antagonists of SLC38A9 as shown herein, provides for a scientific rationale in the therapy of such diseases.

The mTOR complex 1 is composed of Raptor (Uniprot: Q8N122); mTOR (Uniprot: P42345), mLST8 (Uniprot: Q9BVC4), Deptor (Uniprot: Q8TB45) and PRAS40 (Uniprot: Q96B36). Exemplary nucleotide sequences and amino acid sequences of the mTOR complex 1 are shown in SEQ ID NO: 23-52. The terms “mTOR complex 1” and “mTORC1” are used interchangeably herein.

mTOR is a serine/threonine kinase that associate in a large protein complex with RAPTOR, mLST8, Deptor and PRAS40 to form the mTOR complex 1. This complex has a prominent role in controlling cellular growth and metabolism, both in normal and disease conditions mTORC1 integrate signals from growth factors, energy levels and nutrient availability to control critical cellular process, such as protein translation, ribosome biogenesis, lipid synthesis, energy metabolism, lysosome biogenesis and autophagy. Therefore, mTORC1 is at the center of the cell decision of engaging anabolic or catabolic programs.

mTORC1 activity can be measured by monitoring the phosphorylation of its target substrates, such as S6 kinase and/or 4E-BP1 to control protein synthesis or ULK1 for autophagy regulation (as shown in FIG. 4 as well as in references 4, 5, 13 and 14). Aberrant mTORC1 activation can be measured by an increase in phosphorylation of its target substrates, for example S6 kinase and/or 4E-BP1, in disease patient samples when compared the corresponding healthy tissue using for example immunohistochemistry or immunoblot.

Aberrant activation of mTORC1 is known in the art to be implicated in several disease, and in particular metabolic disorders like type 2 diabetes as well as in proliferative malignancies like cancer as described in ref 7, 8, 21 and (Efeyan, A., R. Zoncu, and D. M. Sabatini, Amino acids and mTORC1: from lysosomes to disease. Trends Mol Med, 2012. 18(9): p. 524-33.). Germline or somatic mutations in genes, oncogenes as well as tumor suppressor genes, resulting in increase mTORC1 activity and thereby promoting tumor formation are known in the art (Efeyan, A., R. Zoncu, and D. M. Sabatini, Amino acids and mTORC1: from lysosomes to disease. Trends Mol Med, 2012. 18(9): p. 524-33; Bar-Peled, L., et al., A Tumor suppressor complex with GAP activity for the Rag GTPases that signal amino acid sufficiency to mTORC1. Science, 2013. 340(6136): p. 1100-6.)

(An) exemplary disease(s) associated with mTORC1 activation to be treated in accordance with the present invention is/are (a) proliferative disease(s), (a) metabolic disorder(s), (a) disorder(s) of the immune system, (a) disorder(s) causing premature aging, (an) ophthalmic disorder(s) or (a) neurological disorder(s).

An exemplary disorder of the immune system to be treated in accordance with the present invention is/are autoimmune diseases such as systemic lupus erythematosus (Fernandez, D. and A. Perl, mTOR signaling: a central pathway to pathogenesis in systemic lupus erythematosus? Discov Med, 2010. 9(46): p. 173-8 and Yang, H., et al., Modulation of TSC-mTOR signaling on immune cells in immunity and autoimmunity. J Cell Physiol, 2014. 229(1): p. 17-26.)

An exemplary disorder causing premature aging to be treated in accordance with the present invention is Hutchinson-Gilford progeria syndrome (Graziotto, J. J., et al., Rapamycin activates autophagy in Hutchinson-Gilford progeria syndrome: implications for normal aging and age-dependent neurodegenerative disorders. Autophagy, 2012. 8(1): p. 147-51.)

An exemplary disorder of an ophthalmic disorder to be treated in accordance with the present invention is age-related macular degeneration (Zhao, C. and D. Vollrath, mTOR pathway activation in age-related retinal disease. Aging (Albany N.Y.), 2011. 3(4): p. 346-7).

An exemplary disorder of a neurological disorder to be treated in accordance with the present invention is Huntington's, Parkinson's, Alzheimer's disease (mentioned for example in ref 8).

Proliferative disease(s) to be treated herein is/are (a) cancerous disease(s) or (a) benign proliferative disease(s). An exemplary benign proliferative disease(s) to be treated in accordance with the present invention is tuberous sclerosis.

Preferred herein is the treatment/therapy of (a) cancerous disease(s). Exemplary cancerous disease(s) is/are lung cancer, breast cancer, bladder cancer, pancreatic cancer, ovarian cancer, colon carcinoma, leukemia, lymphoma, melanoma, esophageal cancer or stomach cancer.

The following references disclose that metabolic disorders and cancerous diseases are causally linked to aberrant mTORC1 activation. The references also disclose (animal) models of these diseases. These and other suitable (animal) models are known in the art and can be used to validate that the herein provided antagonists of SLC38A9 can be used in the therapy of these diseases/disorders.

Lung cancer: Engelman, J. A., et al., Effective use of PI3K and MEK inhibitors to treat mutant Kras G12D and PIK3CA H1047R murine lung cancers. Nat Med, 2008. 14(12): p. 1351-6.

Colon carcinoma: APC(Min/+) mice. Koehl, G. E., et al., Rapamycin inhibits oncogenic intestinal ion channels and neoplasia in APC(Min/+) mice. Oncogene, 2010. 29(10): p. 1553-60.

Breast cancer: Chen, Z., et al., mTORC1/2 targeted by n-3 polyunsaturated fatty acids in the prevention of mammary tumorigenesis and tumor progression. Oncogene, 2013.

Pancreatic cancer: Morran, D. C., et al., Targeting mTOR dependency in pancreatic cancer. Gut, 2014.

Melanoma Posch, C., et al., Combined targeting of MEK and PI3K/mTOR effector pathways is necessary to effectively inhibit NRAS mutant melanoma in vitro and in vivo. Proc Natl Acad Sci USA, 2013. 110(10): p. 4015-20.

Ovarian cancer: Wu, R., et al., Preclinical testing of PI3K/AKT/mTOR signaling inhibitors in a mouse model of ovarian endometrioid adenocarcinoma. Clin Cancer Res, 2011. 17(23): p. 7359-72.

Bladder cancer Seager, C. M., et al., Intravesical delivery of rapamycin suppresses tumorigenesis in a mouse model of progressive bladder cancer. Cancer Prev Res (Phila), 2009. 2(12): p. 1008-14.

Leukemia Maude, S. L., et al., Targeting JAK1/2 and mTOR in murine xenograft models of Ph-like acute lymphoblastic leukemia. Blood, 2012. 120(17): p. 3510-8.

esophageal cancer Nishikawa, T., et al., Antiproliferative effect of a novel mTOR inhibitor temsirolimus contributes to the prolonged survival of orthotopic esophageal cancer-bearing mice. Cancer Biol Ther, 2013. 14(3): p. 230-6.

The treatment of metabolic disorder(s) by antagonists of SLC38A9 is envisaged herein. Generally, the metabolic disorder(s) to be treated herein can be characterized by 20% or more body fat in the subject to be treated. (An) exemplary metabolic disorder(s) to be treated in accordance with the present invention is/are overweight (pre-obesity), obesity or diabetes. Preferably, the diabetes is type 2 diabetes.

It is known in the art that activation of mTORC1 is associated with diabetes type 2; Das, A., et al., Mammalian target of rapamycin (mTOR) inhibition with rapamycin improves cardiac function in type 2 diabetic mice: potential role of attenuated oxidative stress and altered contractile protein expression. J Biol Chem, 2014. 289(7): p. 4145-60.

A suitable (animal) model of diabetes type 2 are db/db mice which are known in the art. This and other suitable models can be used to validate that the herein provided antagonists of SLC38A9 can be used in the therapy of metabolic disorders, such as diabetes type 2.

Obesity is a condition where excess body fat accumulates to such an extent that one's health may be affected; see Amer (2010) Biochem and Biophys Res Comm 396, 101-104. Especially in developed countries obesity is increasing and constitutes a major health problem, as obesity also enhances the risk for cardiovascular disease and metabolic disorders such as type 2 diabetes; see Spalding (2008) Nature 453, 783-787.

Overweight and obesity are defined as abnormal or excessive fat accumulation that may impair health. Body mass index (BMI) is a simple index of weight-for-height that is commonly used to classify overweight and obesity in adults. It is defined as a person's weight in kilograms divided by the square of his height in meters (kg/m²).

An “overweight” patient is often defined as having a body mass index (BMI) above 25 kg/m². In context of the present invention, “overweight” is preferably defined as a body mass index (BMI) between 25 to 30 kg/m² and “obesity” is preferably defined as a body mass index (BMI) of higher than 30 kg/m². “Severe obesity” is usually defined as a body mass index (BMI) of 40 kg//m² and higher than 40 kg/m². These definitions are in line with the present definition of the WHO: according to the WHO, a BMI greater than or equal to 25 is overweight and a BMI greater than or equal to 30 is obesity.

According to WHO, raised BMI is a major risk factor for noncommunicable diseases such as cardiovascular diseases (mainly heart disease and stroke), diabetes, musculoskeletal disorders (especially osteoarthritis—a highly disabling degenerative disease of the joints) and some cancers (like endometrial cancer, breast cancer, and colon cancer). The risk for these noncommunicable diseases increases with the increase in BMI. Accordingly, patients prone to suffering from cancer may have the above secondary disorders and diseases.

In one aspect, patients to be treated herein are overweight or obese children. It is known in the art that childhood obesity is associated with a higher chance of obesity, premature death and disability in adulthood. In addition to increased future risks, obese children experience breathing difficulties, increased risk of fractures, hypertension, early markers of cardiovascular disease, insulin resistance and psychological effects. Accordingly, the therapy of these patients (having, for example, childhood obesity) is envisaged in the present invention.

BMI provides the most useful population-level measure of overweight and obesity as it is the same for both sexes and for all ages of adults. However, it should be considered a rough guide because it may not correspond to the same degree of fatness in different individuals. In certain medically indicated cases, it is therefore envisaged that also patients with a BMI below 25 kg/m² can be assessed in accordance with the present invention. In the same vein, not every subject/patient with a high BMI (e.g. between 25 to 30 kg/m² or higher than 30 kg/m²) is an “obese” or “overweight” patient—it is well known that individuals with greater than average muscle mass (e.g. certain athletes (like bodybuilders)) will have a higher BMI without having abnormal or excessive fat accumulation.

Therefore, the patient that is to be treated in accordance with the present invention may be characterized by the presence of 20% or more body fat in the subject/patient. For example, a body fat percentage of 25% or more may be characteristic for an overweight/obese man, and a body fat percentage of 32% or more may be characteristic for an overweight/obese woman. It is known in the art that a person's body fat percentage is the total weight of the person's fat divided by the person's weight.

The body's fat consists of essential body fat and storage body fat. Essential body fat is necessary to maintain life and reproductive functions. Essential fat is usually 3%-5% in men, and 8-12% in women. Storage body fat consists of fat accumulation in adipose tissue, part of which protects internal organs in the chest and abdomen.

The table below describes different percentages that are often used in the art to characterize the percentage of essential fat and the percentage of total fat in men and women:

| Description | Women | Men |
|---|---|---|
| Essential fat | 10-13% | 2-5% |
| Athletes | 14-20% | 6-13% |
| Fitness | 21-24% | 14-17% |
| Average | 25-31% | 18-24% |
| Obese | 32%+ | 25%+ |

The percentage of storage fat or extra fat as denoted herein may be calculated from the above given exemplary values. Yet, it is often difficult to exactly determine the percentage of essential fat and of storage fat. Therefore, the total fat percentage is routinely determined/estimated and used in the art in order to classify a subject/patient as overweight/obese. Appropriate measurement techniques are known in the art and include Near-infrared interactance or Dual energy X-ray absorptiometry (DXA). Also multicompartment models can be used; these models can include DXA measurement of bone, plus independent measures of body water and body volume. Various other components may be independently measured, such as total body potassium. Also in-vivo neutron activation can quantify all the elements of the body and use mathematical relations among the measured elements in the different components of the body (fat, water, protein, etc.) to develop simultaneous equations to estimate total body composition, including body fat. Also body average density measurement can be used to determine a subject/patients body fat percentage: this technique involves the measurement of a person's average density (total mass divided by total volume) and the application of a formula to convert that to body fat percentage. Bioelectrical impedance analysis is also a well known technique to estimate body fat percentage. Also anthropometric methods (measurements made of various parameters of the human body, such as circumferences of various body parts or thicknesses of skinfolds) may be used. Because most anthropometric formulas such as the Durnin-Womersley skinfold method, the Jackson-Pollock skinfold method, and the US Navy circumference method, estimate body density, the body fat percentage is obtained by applying a second formula, such as the Siri or Brozek formula. Further, Skinfold methods may applied and the body fat percentage may even be calculated from the BMI. These and other methods are well known and can be deduced from reviews like Lee (2008) Curr Opin Clin Nutr Metab Care 11(5), 566-572 and Gallagher (2008) Int J Body Compos Res 6(4): 141-148 which are incorporated in their entirety herein.

Preferably, the body fat percentage of a male patient/subject to be treated herein is at least 18%, 19%, 20%, 21%, 22%, 23%, 24% and more preferably, at least 25%. The body fat percentage of a female patient/subject to be treated herein is at least at least 25%, 26%, 27%, 28%, 29%, more preferably 30%, 31% and even more preferably at least 32%. The identification of obese patients according to the body fat percentage (for example determined according to the bioelectrical impedance criterion) may be especially advantageous in individuals having a BMI of below 30 kg/m²; according to the bioelectrical impedance criterion a man may be considered obese in case of a body fat percentage of at least 25% and a woman may be considered obese in case of a body fat percentage of at least 30%; see Frankenfield (2001) Nutrition 17:26-30 which is incorporated in its entirety herein. Upper limits of body fat percentage will have to be calculated on an individual basis; yet, typically body fat percentage does not exceed about 60% even in severely obese subjects/patients.

Further, an obese/overweight patient to be treated herein may have higher levels of triglycerides in the blood of the patient. The recommended level of triglycerides (in a normal range) is in males 40-160 mg/dL and in females 35 to 135 mg/dL. However, in Germany also “higher levels” are tolerated on being normal; e.g. 250 mg/dL. Accordingly, higher levels of triglycerides are preferably above 150 mg/dL, more preferably above 200 mg/dL and most preferably above 250 mg/dL.

Accordingly, the patients to be treated in accordance with the present invention can have overweight, obesity, and/or eating disorders leading to increased BMI/body fat percentage/body weight/body mass as defined herein above. Also envisaged is the therapy of patients with disorders related to higher or pathologically high BMI/body fat percentage/body weight due to the use of drugs (like corticosteroids, antipsychotic drugs, antidepressants, particularly tricyclic antidepressants, oral contraceptives, etc.).

According to the International Statistical Classification of Diseases and Related Health Problems (10th Revision, Version for 2007) issued by the World Health Organization, the following diseases and disorders relate to obesity:

### E66 Obesity

Excludes: adiposogenital dystrophy (E23.6)
   lipomatosis:
      NOS (E88.2)
      dolorosa [Dercum] (E88.2)
   Prader-Willi syndrome (Q87.1)
      E66.0 Obesity due to excess calories
      E66.1 Drug-induced obesity
Use additional external cause code (Chapter XX), if desired, to identify drug.
   E66.2 Extreme obesity with alveolar hypoventilation
Pickwickian syndrome
   E66.8 Other obesity
Morbid obesity
   E66.9 Obesity, unspecified
Simple obesity NOS

In accordance with this invention it is also envisaged that patients to be treated herein can suffer from secondary disorders related to a (pathological) increase of body weight/BMI/body fat percentage (e.g. overweight/obesity). These “secondary disorders” may comprise, but are not limited to diabetes type 2, high blood pressure (hypertension), cardio-vascular diseases, problems with sexual function and disorder of the muscular or bone system, and lipid disorders (such as hypertriglyceridemia and hypercholesterolemia), growth hormone deficiency, partial growth hormone deficiency or neuro-secretory dysfunction of growth hormone secretion. Problems with sexual function may comprise libido problems, penile dysfunction as well as FSAD (Female Sexual Arousal Disorder). Also dyslipidaemia may be a “secondary disorder”.

Secondary disorders of the metabolism linked to higher body weight/body mass/BMI/body fat percentage may also comprise, but are not limited to, glycogen storage diseases, lipid storage diseases (like Gaucher or Niemann Pick), endocrine disorders (like Cushings, hypothyroidism, insulinomas, lack of growth hormone, diabetes, adrenogenital syndrome, diseases of the adrenal cortex), tumors and metastases (such as craniopharyngeomas), Prader-Willi syndrome, Down syndrome and genetic diseases and syndromes (like, e.g., hyperlipoproteinemias, hypothalamic disorders, Frohlich syndrome or empty sella syndrome).

Diabetes mellitus type 2 is a condition relating to non-insulin-dependent diabetes mellitus. Non-insulin-dependent diabetes mellitus is a risk factor/secondary disorder in context of the present invention. Diabetes mellitus type 2 results from insulin resistance, a condition in which cells fail to use insulin properly, sometimes combined with an absolute insulin deficiency. This form was previously referred to as non insulin-dependent diabetes mellitus (NIDDM) or “adult-onset diabetes”.

Non-insulin-dependent diabetes mellitus can be classified in accordance with the ICD-10 version:2010 of the World Health Organization (WHO) as follows:

E11 Non-insulin-dependent diabetes mellitus

### Incl.:

diabetes (mellitus)(nonobese)(obese):
adult-onset
maturity-onset
nonketotic
stable
type II
   non-insulin-dependent diabetes of the young

### Excl.:

diabetes mellitus (in):
malnutrition-related (E12.-)
neonatal (P70.2)
pregnancy, childbirth and the puerperium (O24.-)
   glycosuria:
NOS (R81)
renal (E74.8)
   impaired glucose tolerance (R73.0)
   postsurgical hypoinsulinaemia (E89.1)

The present invention provides antagonists of SLC38A9. These antagonists can be used as a medicament, i.e. the antagonists of SLC38A9 provided and described herein are for use in medicine (e.g. for use in the therapy/treatment of a disease, in particular a disease associated with mTORC1 activation). The terms “medicament” and “pharmaceutical composition” are used interchangeably herein. Accordingly, definitions and explanations provided herein in relation to “pharmaceutical compositions”, apply, mutatis mutandis, to the term “medicament”.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related in a subject which may be predisposed to the disease; (b) inhibiting the disease, i.e. arresting its development; or (c) relieving the disease, i.e. causing regression of the disease.

An “individual”, “patient” or “subject” for the purposes of the present invention includes both humans and other animals, particularly mammals, and other organisms. Thus, the methods are applicable to both human therapy and veterinary applications. Preferably, the “individual”, “patient” or “subject” is a mammal, and most preferably the “individual”, “patient” or “subject” is human.

The following relates to “antagonist of SLC38A9” provided and to be used in accordance with the present invention.

The terms “antagonist of SLC38A9” and “inhibitor of SLC38A9” are used interchangeably herein.

The terms “antagonist of SLC38A9” or “inhibitor of of SLC38A9” means in context of the present invention a compound capable of fully or partially preventing or reducing the physiologic activity and/or expression level of SLC38A9. The terms “antagonist” or “inhibitor” are used interchangeably herein. It is envisaged herein that the antagonist of SLC38A9 is a selective antagonist of SLC38A9.

In the context of the present invention said antagonist may, therefore, prevent, reduce, inhibit or inactivate the physiological activity of SLC38A9 e.g. upon binding of said compound/substance (i.e. antagonist/inhibitor) to said SLC38A9. As used herein, the term “antagonist” also encompasses competitive antagonists, (reversible) non-competitive antagonists or irreversible antagonist, as described, inter alia, in Mutschler, “Arzneimittelwirkungen” (1986), Wissenschaftliche Verlagsgesellschaft mbH, Stuttgart, Germany. Such an inhibition can be measured by determining substrate turnover.

An “antagonist” or “inhibitor” of SLC38A9 may also be capable of preventing the function of a SLC38A9 by preventing/reducing the expression of the nucleic acid molecule encoding for said SLC38A9. Thus, an antagonist/inhibitor of SLC38A9 may lead to a decreased expression level of SLC38A9 (e.g. decreased level of SLC38A9 mRNA and/or of SLC38A9 protein); this may be reflected in a decreased SLC38A9 activity. The decreased activity and/or expression level can be measured/detected by known methods which are also described herein.

An “antagonist/inhibitor of SLC38A9” may, for example, interfere with transcription of (an) SLC38A9 gene(s), processing (e.g. splicing, export from the nucleus and the like) of the gene product(s) (e.g. unspliced or partially spliced mRNA) and/or translation of the gene product (e.g. mature mRNA). The “antagonist/inhibitor of SLC38A9” may also interfere with further modification (like glycosylation or phosphorylation) of the polypeptide/protein encoded by the SLC38A9 gene(s) and thus completely or partially inhibit the activity of the a SLC38A9 protein(s) as described herein above. Furthermore, the “antagonist/inhibitor of a SLC38A9” may interfere with interactions of the SLC38A9 protein(s) with other proteins (thus, for example, interfering with the activity of complexes involving SLC38A9 protein(s)) or, in general, with its synthesis, e.g. by interfering with upstream steps of SLC38A9 expression or with signalling pathways in which the SLC38A9 is involved. Depending on the mode of action, such antagonists may, for example, be denoted “sequestering antagonists” or “signalling antagonists”.

In sum, the herein described SLC38A9 antagonist/inhibitor will, accordingly, lead to a decrease or reduction of SLC38A9 expression level and/or activity, and thereby reduce its contribution to the development, proliferation or progress of a disease associated with mTORC1 activation as defined herein.

The antagonist(s) may be (a) (small) binding molecule, (a) small molecule drug(s), siRNA, shRNA, miRNA, dsRNA, stRNA or antisense molecules.

For example, the binding molecule(s) can be (an) aptamer(s) and/or (an) intramer(s). Also envisaged are extracellular binding-partners.

It is envisaged herein that the binding molecule antagonizing SLC38A9 specifically binds to SLC38A9, particularly to the SLC38A9 protein (or a part thereof, like the cytoplasmic region or portions or conservative domains/motivs thereof) as defined herein.

For example, aptamers/intramers targeting the N-terminal cytoplasmic region as defined here can be used herein as antagonists of SLC38A9.

For example, aptamers/intramers to be used herein can specifically target or bind to the following N-terminal cytoplasmic region of SLC38A9 as shown in SEQ ID NOs: 22, 54, 56, 58, 60, or 62. For example, aptamers/intramers to be used herein can specifically target or bind to the following transmembrane region of SLC38A9 as shown in SEQ ID NOs: 68, 70, 72 or 74.

It is invisaged herein that the aptamers/intramers can specifically target/bind to (functional) fragments or (functional) derivatives of the SLC38A9 proteins as defined herein, for example also to polypeptides having at least 70% or more identity to herein provided SLC38A9 protein(s).

Accordingly, the present invention relates to the use of these aptamers/intramers in particular in the therapeutic methods of the present invention.

(A) small molecule drug(s) to be used herein as antagonist of SLC38A9 refers to an (organic) low molecular weight (<900 Daltons) compound. Small molecules can help to regulate a biological process and have usually a size in the order of 10⁻⁹ m. Antagonists to be used herein, like small molecules (drugs), can, for example, be identified by screening compound libraries, for example Enamine, Chembridge or Prestwick chemical libraries.

Furthermore, exemplary antagonists of SLC38A9 provided and used herein are siRNA, shRNA, miRNA, dsRNA, stRNA, or antisense molecule targets a nucleic acid molecule having a sequence encoding SLC38A9. The nucleic acid molecule having a sequence encoding SLC38A9 is especially mRNA as defined herein.

The present invention relates to and provides in particular an siRNA or shRNA specifically targeting the nucleic acid encoding the SLC38A9 protein(s), whereby the nucleic is especially mRNA as defined herein below.

Antagonist(s)/inhibitor(s) of SLC38A9 which are nucleic acids, such as siRNAs, shRNAs, antisense molecules and the like can readily be prepared by known techniques using, for example, the following target sequences. For example, siRNAs, shRNAs and the like to be employed herein can comprise or consist of an RNA sequence corresponding to one of the target sequences further described below. The term “RNA sequence corresponding to” means in this context that the RNA sequence is identical to one of the target sequences below, if necessary with the exception that the tymidine (T) residues of the target sequence is replaced by a uracil (U) residue. It is understood that siRNAs, shRNAs usually comprise one strand that is (partially) complementary to the target sequence.

The siRNA can consist of or comprise a nucleic acid molecule comprising at least eight (or ten) contiguous bases. For example, the siRNA, shRNA and the like can comprise at least eight (or ten) contiguous bases of an RNA sequence corresponding to one of the target sequences below as defined above. The siRNA, shRNA and the like can consist or comprise of ten contiguous bases of an RNA sequence corresponding to one of the target sequences below as defined above. For example, an antagonizing siRNA to be used herein can comprise a nucleic acid molecule comprising at least eight contiguous bases having a sequence as shown in the sequence of SEQ ID NO: 5, 6, 7 or 8.

Up to 10% of the contiguous bases of the herein provided siRNAs or shRNAs and the like can be non-complementary (to the target sequence). The siRNA can further comprise at least one base at the 5′ end and/or at least one base at the 3′ end. The siRNA can consist of a molecule as shown in SEQ ID NO: 5, 6, 7 or 8 and/or an RNA molecule (partially) complementary to the sequence as shown in SEQ ID NO: 5, 6, 7 or 8. Preferably, the siRNA consists of a molecule as shown in SEQ ID NO: 5, 6, 7 or 8 and an RNA molecule (partially) complementary to the sequence as shown in SEQ ID NO: 5, 6, 7 or 8.

compl-rev:
SEQ ID NO: 5
UUACCGUAUCCUUCAGUGU
compl-rev:
SEQ ID NO: 6
UAUUCAUAGGUCCAGGAUC
compl-rev:
SEQ ID NO: 7
UAUACACAUAGCACUCUUC
compl-rev:
SEQ ID NO: 8
UAACCCUCUGAAUGACAUG

Corresponding RNA molecule complementary to the sequence as shown in SEQ ID NO: 5, 6, 7 or 8 are as follows:

SiRNA sequence 1 (sense sequence):
(SEQ ID NO: 63)
ACACUGAAGGAUACGGUAA
→ target nt 530-548 of human SLC38A9 mRNA
(NM_173514.3)
SiRNA sequence 2:
(SEQ ID NO: 64)
GAUCCUGGACCUAUGAAUA
→ target nt 262-280 of human SLC38A9 mRNA
(NM_173514.3)
SiRNA sequence 3:
(SEQ ID NO: 65)
GAAGAGUGCUAUGUGUAUA
→ target nt 478-496 of human SLC38A9 mRNA
(NM_173514.3)
SiRNA sequence 4:
(SEQ ID NO: 66)
CAUGUCAUUCAGAGGGUUA
→ target nt 355-373 of human SLC38A9 mRNA
(NM_173514.3)

A preferred target sequence of the nucleic acids antagonists as defined above (e.g. siRNA or shRNA and the like) can be selected from the group consisting of

(a) a nucleic acid encoding a polypeptide comprising an amino acid sequence as depicted in SEQ ID NO: 3;
(b) a nucleic acid comprising a nucleotide sequence as depicted in SEQ ID NO: 4;
(c) a nucleic acid hybridizing under stringent conditions to the complementary strand of the nucleic acid as defined in (a) or (b);
(d) a nucleic acid comprising a nucleotide sequence with at least 70% identity to the nucleotide sequence of the nucleic acids of any one of (a) to (c); and
(e) a nucleic acid comprising a nucleotide sequence which is degenerate as a result of the genetic code to the nucleotide sequence of a nucleic acid of any one of (a) to (d).

Particular target sequences (especially for siRNA) are shown in SEQ ID NO. 63, SEQ ID NO. 64, SEQ ID NO. 65, or SEQ ID NO. 66. These target sequences correspond to nt 530-548 of human SLC38A9 mRNA (NM_173514.3), nt 262-280 of human SLC38A9 mRNA (NM_173514.3), target nt 478-496 of human SLC38A9 mRNA (NM_173514.3), and nt 355-373 of human SLC38A9 mRNA (NM_173514.3), respectively. These sequences represent target sequence of the nucleic acids antagonists as defined above (e.g. siRNA or shRNA and the like, preferably of siRNA). An exemplary SLC38A9 mRNA sequence is shown in SEq ID NO: 4, so that the above target sequences and corresponding target sequences in variants or isoforms of that specific sequence can easily be identified.

These siRNA can be used alone or in combination as antagonists of SLC38A9 in accordance with the present invention.

Concerning the length usually siRNA are of 19 to 21 nt (or in case of a complex of sense and antisense strand of 19 to 21 bp). For silencing it is crucial that bases from 2 to 8 (seed region) of the siRNA (i.e. in this context of the antisense strand) have perfect base pairing with the target sequence.

The shRNA used and provided in the experiments has the following full length sequence:

(SEQ ID NO: 113)
CCGGGCCTTGACAACAGTTCTATATCTCGAGATATAGAACTGTTGTCAA
GGCTTTTTTG.
(The mature antisense sequence is highlighted in
bold letters)

The mature antisense sequence of the shRNA used and provided in the experiments is: ATATAGAACTGTTGTCAAGGC (SEQ ID NO: 114). SEQ ID NO: 113 and 114 show DNA sequences corresponding to the RNA sequence of the shRNA as shown in SEQ ID NO. 9 and 10, respectively.

The corresponding sense target sequence is: GCCTTGACAACAGTTCTATAT (SEQ ID NO: 11). This corresponds to nt 1931-1951 of human SLC38A9 mRNA (NM_173514.3). The RNA sequence corresponds to the sequence shown in SEQ ID NO: 11 with the exception that the thymidine residues are replaced by uracil residues. Accordingly, these sequences represent target sequence of the nucleic acids antagonists as defined above (e.g. siRNA or and the like, preferably of shRNA).

The shRNA can comprise (or consist of) a nucleic acid molecule comprising (or consisting of) (at least eight contiguous nucleotides having) a sequence as shown in the sequence of SEQ ID NO: 9 or 10. In one embodiment, the shRNA provided and to be used herein consists of a nucleic acid sequence as shown in SEQ ID NO: 9. In another embodiment, the mature antisense sequence of the shRNA to be used and provided consists of a nucleic acid sequence as shown in SEQ ID NO: 10.

The inhibitor of SLC38A9 may be administered as a single agent (i.e. in form of a monotherapy) or in form of a combination therapy, for example, conventional therapies like chemo- or radiotherapy for cancers, diet for obesity and methformin or insulin for diabetes type 2.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of pharmaceutical composition administered to an individual will, inter alia, depend on the nature of the compound.

For example, if said inhibitor is a small molecule, the total (pharmaceutically) effective amount of the inhibitor in the pharmaceutical composition administered orally per dose will be in the range of about 50 mg inhibitor per day to 1000 mg inhibitor per day of patient, although, as noted above, this will be subject to therapeutic discretion. More preferably, this dose is at least 50 mg inhibitor per day, and most preferably for humans between about 50 mg and 600 mg inhibitor per day. For example, an inhibitor may be administered at a dose of 15 mg/kg body weight per day. If given continuously, the inhibitor is typically administered at a dose rate of about 50 mg per day to about 600 mg per day. An intravenous bag solution may also be employed. The length of treatment needed to observe changes and the interval following treatment for responses to occur appears to vary depending on the desired effect. The particular amounts may be determined by conventional tests which are well known to the person skilled in the art. The length of treatment needed to observe changes and the interval following treatment for responses to occur appears to vary depending on the desired effect. The particular amounts may be determined by conventional tests which are well known to the person skilled in the art.

The administration of the herein provided compositions may, inter alia, comprise an administration twice daily, every day, every other day, every third day, every forth day, every fifth day, once a week, once every second week, once every third week, once every month, etc.

For example, if said compound is a (poly)peptide or protein the total pharmaceutically effective amount of pharmaceutical composition administered parenterally per dose will be in the range of about 1 μg protein/kg/day to 15 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

Pharmaceutical compositions of the invention may be administered orally, rectally, parenterally, intracisternally, intravaginally, intraperitoneally, topically (as by powders, ointments, drops or transdermal patch), bucally, or as an oral or nasal spray.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

Inhibitors for use in accordance with the present invention are described and provided herein. Also the use of inhibitors yet to be generated or known compounds to be tested for their inhibiting activity is envisaged in context of the present invention.

Therefore, the present invention provides a method for assessing the activity of a candidate molecule suspected of being an antagonist of SLC38A9 as defined and provided herein comprising the steps of:

(a) contacting a cell, tissue or a non-human animal comprising SLC38A9 with said candidate molecule;
(b) detecting a decrease in activity of said SLC38A9; and
(c) selecting a candidate molecule that decreases activity of said SLC38A9.

A decrease of the SLC38A9 activity can indicate the capacity of the selected molecule to antagonise mTORC1.

Also a decrease in the (expression) level can indicate useful inhibitors of SLC38A9. Accordingly, the term “activity” above can comprise and relate to the “expression level” and vice versa.

The present invention relates to a method for assessing the (expression) level of a candidate molecule suspected of being an antagonist of SLC38A9 as defined and provided herein comprising the steps of:

(a) contacting a cell, tissue or a non-human animal comprising SLC38A9 with said candidate molecule;
(b) detecting a decrease in the (expression) level of said SLC38A9; and
(c) selecting a candidate molecule that decreases the (expression) level of said SLC38A9.

A decrease of the SLC38A9 (expression) level can indicate the capacity of the selected molecule to antagonise mTORC1.

All definitions and explanations provided herein above, inter alia, in relation to “SLC38A9” (and related compounds), “antagonist”, “activity” and the like, apply mutatis mutandis in the context of these methods for assessing the activity (or (expression) level) of a candidate molecule suspected of being an antagonist of SLC38A9.

The SLC38A9 can be any of the SLC38A9 proteins/polypeptides as defined herein above or any of the nucleic acids (particularly mRNAs) as defined herein, which encode the SLC38A9 proteins/polypeptides.

The following exemplary assays can be used in the determination that a candidate molecule is indeed an antagonist of SLC38A9 to be used in accordance with the present invention: assays monitoring transport activities using recombinant SLC38A9 protein and inserted into liposomes to obtain proteoliposomes monitoring transport of amino acids (either radioactive or detected by alternative methods), assays using microinjection in Xenopus oocytes to express SLC38A9 and monitoring transport as above, assays monitoring physical engagement of SLC38A9 using biophysical methods, such as surface plasmon resonance, NMR, alphascreen, fluorescence interference.

Such exemplary assays are described herein below in more detail:

The following assay can be used to identify antagonists of SLC38A9 (like SLC38A9 activity/function):

### 1. In Vitro Proteoliposome Transport Assay

An in vitro proteoliposome transport assay used to determine SLC38A9 ability to transport amino acids can be used for screening of candidate molecules able to compete with transport of radio- or fluorescently-labelled amino acids. Such assays are known and disclosed in the art, inter alia, in Scalise M, Pochini L, Giangregorio N, Tonazzi A, Indiveri C. Proteoliposomes as tool for assaying membrane transporter functions and interactions with xenobiotics.Pharmaceutics. 2013 Sep. 18; 5(3):472-97. doi: 10.3390/pharmaceutics5030472, incorporated herein by reference)

A corresponding proteoliposome transport assay is shown in the appended example (see FIG. 3D) and described in the corresponding method section. This transport assay is used to test candidate molecules. Candidate molecules are screened for the ability to inhibit amino acid transport, for example by competing or inhibiting transport of labelled glutamine.

### 2. Overexpression of SLC38A9 Wildtype or Mutant of Lysosomal Targeting Sequences in SLC38A9

Overexpression of SLC38A9 wildtype or mutant of lysosomal targeting sequences in SLC38A9 can be used to localize the transporter to plasma membrane of human cell lines and screen for inhibitor of its transport activity similarly as described in Wetli, H. A., P. D. Buckett, and M. Wessling-Resnick, Small-molecule screening identifies the selanazal drug ebselen as a potent inhibitor of DMT1-mediated iron uptake. Chem Biol, 2006. 13(9): p. 965-72, which is incorporated herein by reference). Candidate molecules can be screened for the ability to influence uptake of labeled amino acids or unlabeled amino acids using metabolomic.

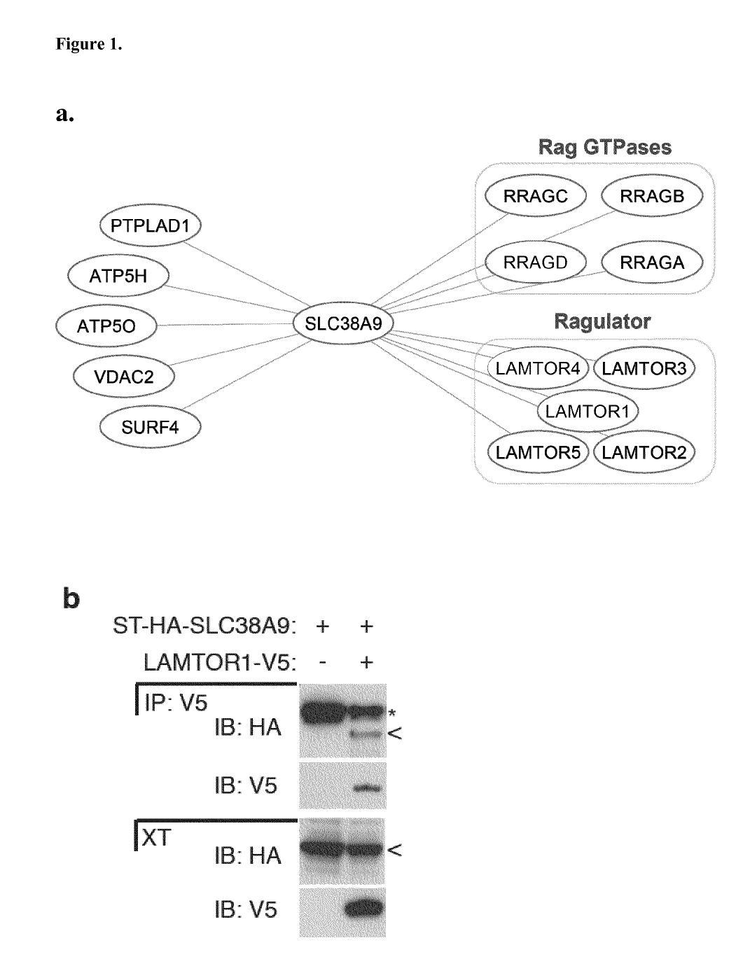

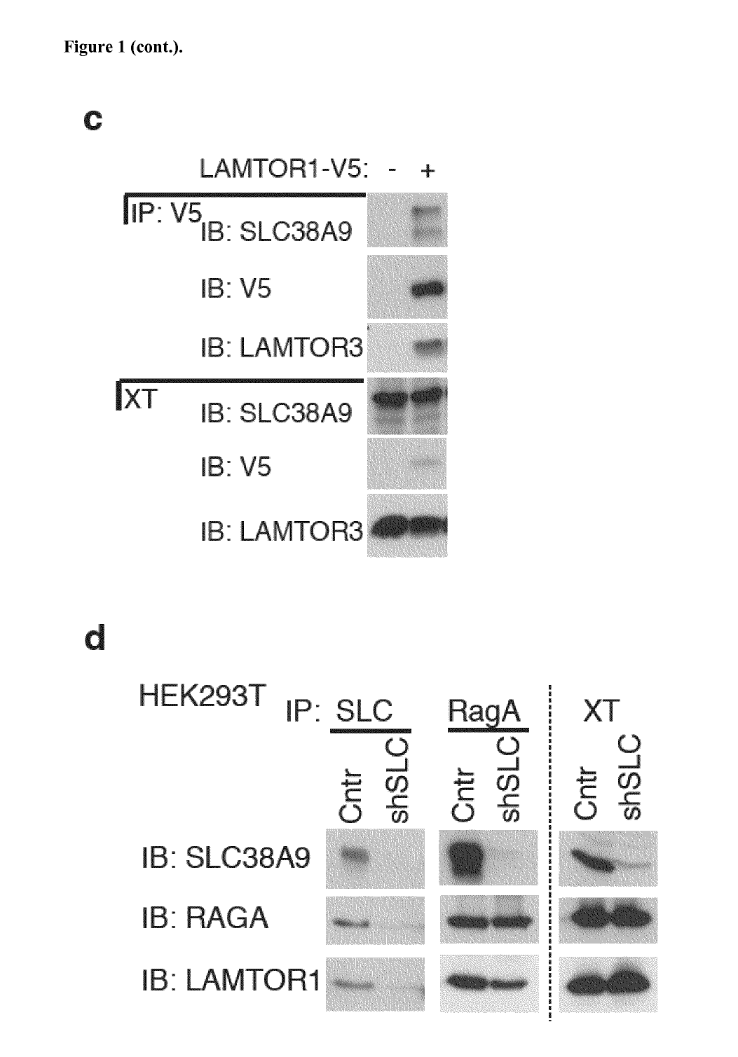

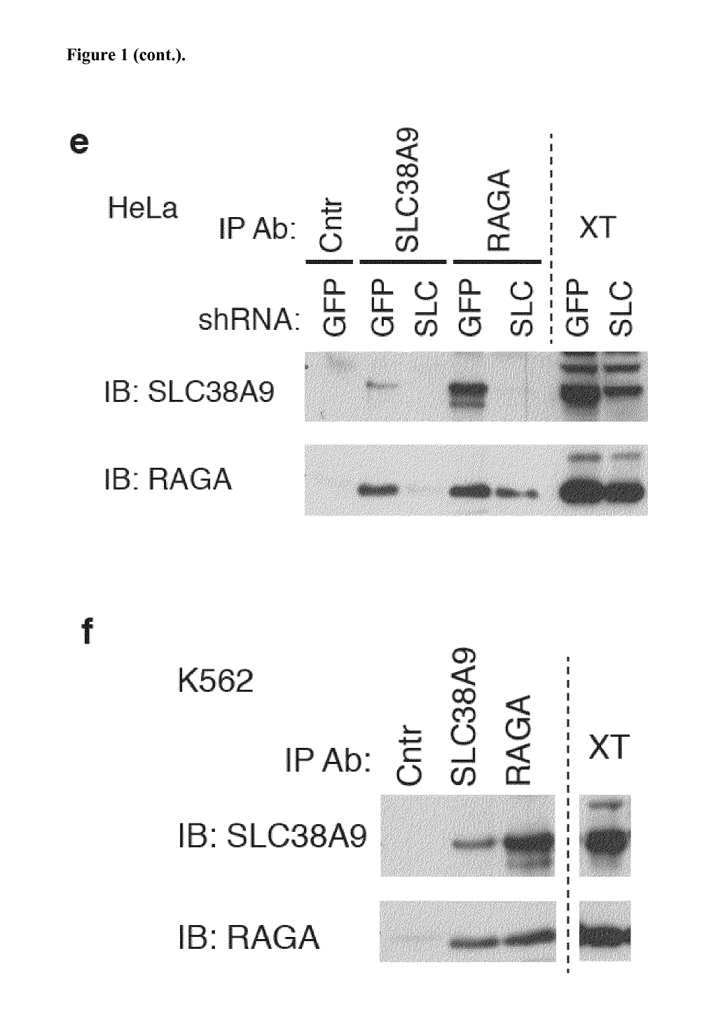

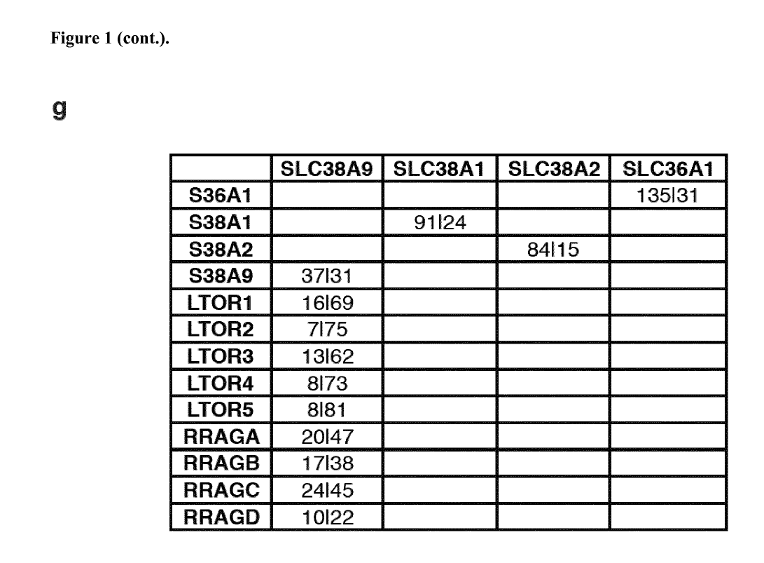

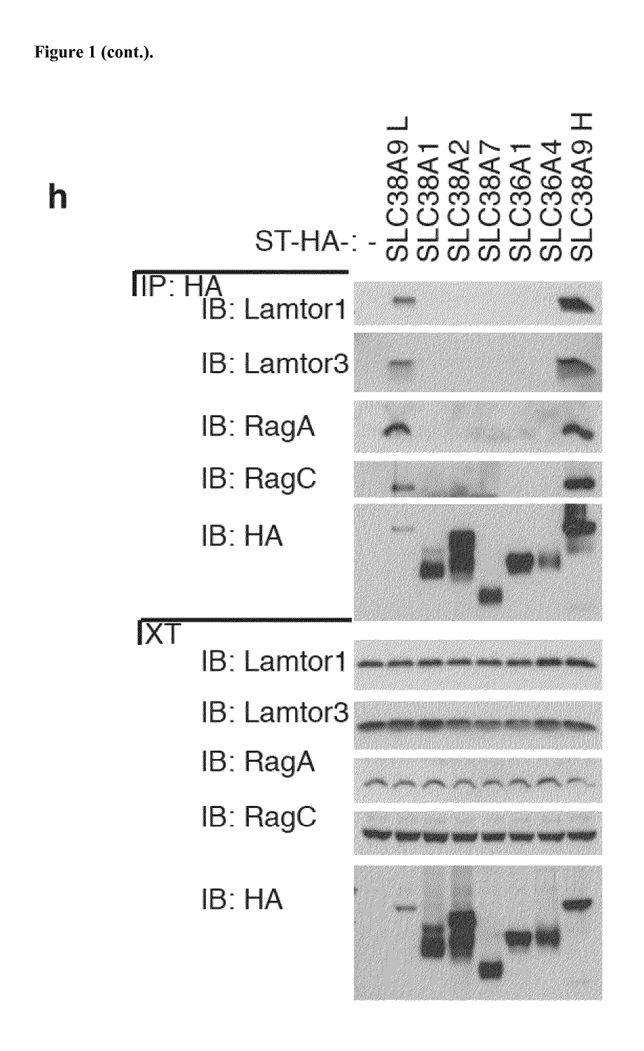

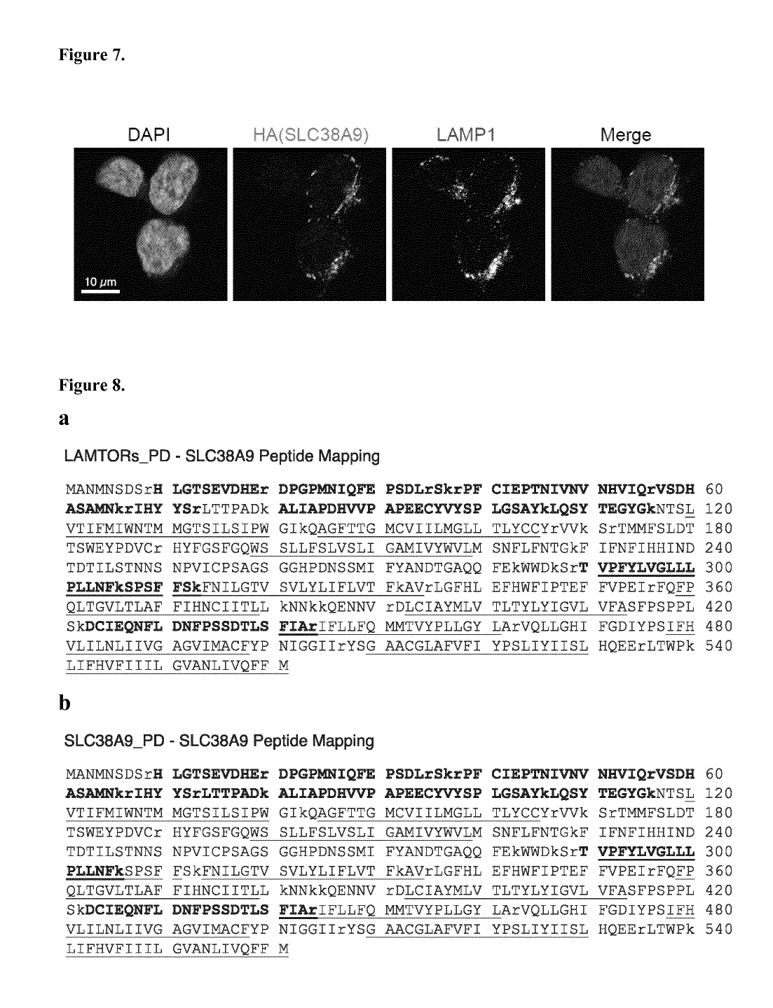

Cell lines stably expressing wild type SLC38A9 are generated. High expressing clones are be selected and tested for plasma membrane localization. In parallel, potential lysosomal localization motifs, such as dileucine motifs present in the C terminal region of SLC38A9 or glycosylation site, will be mutagenized singularly or in combination. Localisation of mutant constructs will be assessed by transient transfection in HEK293T cells followed by immunostaining as described in the method section related to FIG. 1I and FIG. 7. Construct showing plasma membrane localization is selected for verification of retained transport activity in the proteoliposome system and for generation of stable expression in HEK293T, HeLa or other suitable cell lines. SLC38A9-dependent transport of labeled amino acids in these stable expressing cell lines is assessed and candidate molecules are tested for their ability to reduce import of labeled or unlabelled amino acid. SLC38A9 cells is pretreated with a candidate molecule or vehicle control and then incubated with amino acids. In case of labeled amino acids (radio-, fluorescenty-labelled or similar), cells are washed and intracellular accumulation is measured and compared to cells treated with vehicle control. In case of unlabeled amino acids, their extracellular and intracellular concentration concentration is measured by metabolomic.

### 3. In Vitro Binding Assay to Monitor the Interaction of Recombinant Ragulator/RAG GTPase Proteins to the N Terminal Region of SLC38A9

An in vitro binding assay to monitor the interaction of recombinant Ragulator/RAG GTPase proteins to the N terminal region of SLC38A9 can be used. Candidate molecules are screened for the ability to interfere with this interaction similarly as described in (Watson, V. G., et al., Development of a high-throughput screening-compatible assay for the discovery of inhibitors of the AF4-AF9 interaction using AlphaScreen technology. Assay Drug Dev Technol, 2013. 11(4): p. 253-68, which is incorporated herein by reference).)

Recombinant Ragulator/RAG GTPase proteins and the N terminal region of SLC38A9 are produced and used to set up an in vitro binding assay allowing to monitor direct interaction between Ragulator/RAG GTPase protein and SLC38A9. Using this interaction, in vitro binding assay such as AlphaScreen (Amplified Luminescent Proximity Homogeneous Assay Screen) or ELISA (Enzyme-Linked Immunosorbent Assay) are used to screen for candidate molecules able to inhibit binding. To perform AlphaScreen, recombinant biotinylated N terminal region of SLC38A9 and FLAG-tagged Ragulator/Rag interactor are produced. Interaction is detected by AlphaScreen upon addition of streptavidin-coated donor beads and anti-FLAG-coated acceptor beads (AlphaScreen FLAG detection kit, Perkin Elmer). Candidate molecules are then tested for the ability to inhibit the interaction using this assay.

The three approaches described above are used to identify antagonists of SLC38A9 ability in order to inhibit mTORC1 activity. These candidate molecules are then tested for the ability to inhibit cell growth and amino acid induced mTORC1 activity following, for example, the experimental setting used in FIGS. 4A, 4C and 4D and described in the corresponding method section. Cells are treated with candidate antagonist, starved and restimulated with amino acids Inhibition of mTORC1 is monitored by detection of phosphorylated S6 kinase, an established substrate of mTOR kinase, by immunoblot; see references 4, 5, 13, 14, which are incorporated herein by reference. Antagonists with activity in these in vitro system are tested in specific in vivo disease models described herein.

The nucleic acid sequence encoding for orthologous/homologous/identical (and thus related) sequences of the herein provided SLC38A9 is at least 70% homologous/identical to the nucleic acid sequence as, inter alia, shown in SEQ ID NO. 1, 2, 4, 12, 13, 15, 16, 18, 19, 21, 53, 55, 57, 59, 61, 67, 69, 71 or 73 (preferably SEQ ID NO. 1, 2 or 4). More preferably, the nucleic acid sequence encoding orthologous/homologous/identical (and thus related) sequences of the herein provided SLC38A9 is at least 75%, 80%, 85%, 90%, 91%, 92%, 93%, 94%, 95%, 96%, 97% or 98% homologous/identical to the nucleic acid sequence as, inter alia, shown in SEQ ID NOs. 1, 2, 4, 12, 13, 15, 16, 18 19, 21, 53, 55, 57, 59, 61, 67, 69, 71 or 73 (preferably SEQ ID NO. 1, 2 or 4), wherein the higher values are preferred. Most preferably, the nucleic acid sequence encoding for orthologous/homologous/identical (and thus related) sequences of the herein provided SLC38A9 is at least 99% homologous/identical to the nucleic acid sequence as, inter alia, shown in SEQ ID NOs. 1, 2, 4, 12, 13, 15, 16, 18, 19, 21, 53, 55, 57, 59, 61, 67, 69, 71 or 73 (preferably SEQ ID NO. 1, 2 or 4). The term “orthologous protein” or “orthologous gene” as used herein refers to proteins and genes, respectively, in different species that are similar to each other because they originated from a common ancestor.

Hybridization assays for the characterization of orthologs or other related sequences of known nucleic acid sequences are well known in the art; see e.g. Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989).

The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

In accordance with the present invention, the terms “homology” or “percent homology” or “identical” or “percent identity” or “percentage identity” or “sequence identity” in the context of two or more nucleic acid sequences refers to two or more sequences or subsequences that are the same, or that have a specified percentage of nucleotides that are the same (preferably at least 70% identity, more preferably at least 75%, 80%, 85%, 90%, 91%, 92%, 93%, 94%, 95%, 96%, 97% or 98% identity, most preferably at least 99% identity), when compared and aligned for maximum correspondence over a window of comparison (preferably over the full length), or over a designated region as measured using a sequence comparison algorithm as known in the art, or by manual alignment and visual inspection. Sequences having, for example, 75% to 90% or greater sequence identity may be considered to be substantially identical. Such a definition also applies to the complement of a test sequence. Preferably the described identity exists over a region that is at least about 15 to 25 nucleotides in length, more preferably, over a region that is at least about 50 to 100 nucleotides in length and most preferably, over a region that is at least about 800 to 1200 nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. The BLOSUM62 scoring matrix (Henikoff (1989) PNAS 89:10915) uses alignments (B) of 50, expectation (E) of 10, M=5, N=4, and a comparison of both strands.

In order to determine whether an nucleotide residue in a nucleic acid sequence corresponds to a certain position in the nucleotide sequence of e.g. SEQ ID NOs. 1, 2, 4, 12, 13, 15, 16, 18, 19, 21, 53, 55, 57, 59, 61, 67, 69, 71 or 73 (preferably SEQ ID NO. 1, 2 or 4), respectively, the skilled person can use means and methods well-known in the art, e.g., alignments, either manually or by using computer programs such as those mentioned herein. For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score, which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those, which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

The explanations and definitions given herein above in respect of “homology/identity of nucleic acid sequences” apply, mutatis mutandis, to “amino acid sequences” of members SLC38A9, in particular an amino acid sequence as depicted in SEQ ID NO: 3 (SLC38A9 isoform 1), SEQ ID NO: 14 (SLC38A9 isoform 2), SEQ ID NO: 17 (SLC38A9 isoform 3), SEQ ID NO: 20 (SLC38A9 isoform 4), or, in relation to the cytoplasmic region, SEQ ID NOs. 22, 54, 56, 58, 60 or 62, or, in relation to the transmembrane region, SEQ ID NOs: 68 (isoform 1), 70 (isoform 2), 72 (isoform 3) or 74 (isoform 4).

In one embodiment, the polypeptide to be used in accordance with the present invention has at least 70% homology/identity to a SLC38A9 protein/polypeptide having the amino acid sequence as, for example, depicted in SEQ ID NO: 3, 14, 17, 20. 22, 54, 56, 58, 60, 62, 68, 70, 72 or 74, (preferably SEQ ID NO: 3). More preferably, the polypeptide has at least 80%, 85%, 90%, 91%, 92%, 93%, 94%, 95%, 96%, 97% or 98% homology/identity to a SLC38A9 protein/polypeptide having the amino acid sequence as, for example, depicted in SEQ ID NO: 3, 14, 17, 20, 22, 54, 56, 58, 60, 62, 68, 70, 72 or 74, (preferably SEQ ID NO: 3), respectively, wherein the higher values are preferred. Most preferably, the polypeptide has at least 99% homology to a SLC38A9 protein/polypeptide having the amino acid sequence as, for example, depicted in SEQ ID NO: 3, 14, 17, 20, 22, 54, 56, 58, 60, 62, 68, 70, 72 or 74, (preferably SEQ ID NO: 3), respectively.

The terms “complement”, “reverse complement” and “reverse sequence” referred to herein are described in the following example: For sequence 5′AGTGAAGT3′, the complement is 3′TCACTTCAS′, the reverse complement is 3′ACTTCACTS' and the reverse sequence is 5′ TGAAGTGA3′.

The following relates to (transgenic) cell(s), (transgenic) tissue(s) or (transgenic) non-human animal(s) to be used and provided herein. These cell(s), tissue(s) or (animals) are, for example, useful in the herein provided screening assays, like assays for assessing the activity of candidate molecules suspected of being an antagonist of SLC38A9. In particular transgenic cell(s), transgenic tissue(s) or (a) transgenic non-human animal(s) having or comprising the nucleic acid as described and explained herein (or a vector comprising same), e.g. a nucleic acid comprising a sequence encoding SLC38A9 as defined herein are useful for such purpuses. Accordingly, such (transgenic) cell(s), (transgenic) tissue(s) or (a) (transgenic) non-human animal(s) can be used for screening and/or validation of a medicament for the treatment of a disease associated with mTORC1 activation.

The term “cell” as used in this context may also comprise a plurality of cells as well as cells comprised in a tissue. The cell to be used in the screening or validation method may be obtained from samples from a (transgenic) non-human animal or human suffering from a disease associated with mTORC1 activation. The cell (e.g. a tumor cell and the like) may also be obtained or derived from patient samples (e.g. biopsies), in particular a biopsy/biopsies from a patient/subject suffering from a disease associated with mTORC1 activation. Accordingly, the cell may be a human cell. Again, such a cell to be used in the present screening or validation methods may be comprised in a tissue or tissue sample, like in a sample biopsy.

The used non-human animal or cell may be transgenic or non transgenic. “Transgenic” in this context particularly means that SLC38A9 as described or defined herein is (over-) expressed and/or that the activity of SLC38A9 (protein) is present (or increased).

A preferred (transgenic) non-human animal or (transgenic) cell in context of the invention suffers from a disease associated with mTORC1 activation.

The term “transgenic non-human animal” or “transgenic cell” as used herein refers to a non-human animal or cell, not being a human, that comprises genetic material different from the genetic material of a corresponding wild-type animal/cell. “Genetic material” in this context may be any kind of a nucleic acid molecule, or analogues thereof, for example a nucleic acid molecule, or analogues thereof as defined herein. “Different” in this context means additional or fewer genetic material with respect to the genome of the wild-type animal/cell and/or rearranged genetic material, i.e. genetic material present at a different locus of the genome with respect to the genome of the wild-type animal/cell. An overview of examples of different expression systems to be used for generating transgenic cell/animal is, for instance, contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440).

In a preferred embodiment, the (transgenic) non-human animal or (transgenic) cell is or is derived from a mammal. Non-limiting examples of the (transgenic) non-human animal or derived (transgenic) cell are selected from the group consisting of a mouse, a rat, a rabbit, and a guinea pig.

The present invention also relates to a vector comprising the nucleic acid molecule of the present invention.

Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook et al. (loc cit.) and Ausubel, Current Protocols in Molecular Biology, Green Publishing Associates and Wiley Interscience, N.Y. (1989), (1994). Alternatively, the polynucleotides and vectors of the invention can be reconstituted into liposomes for delivery to target cells. As discussed in further details below, a cloning vector was used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

Preferably said vector comprises a nucleic acid sequence which is a regulatory sequence operably linked to said nucleic acid sequence defined herein.

The term “regulatory sequence” refers to DNA sequences, which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the recited vector is preferably an expression vector. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the P_{L}, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements, which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the recited nucleic acid sequence and are well known in the art; see also the appended Examples. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pCDM8, pRc/CMV, pcDNA1, pcDNA3 (In-vitrogene), pEF-DHFR, pEF-ADA or pEF-neo (Mack et al. PNAS (1995) 92, 7021-7025 and Raum et al. Cancer Immunol Immunother (2001) 50(3), 141-150) or pSPORT1 (GIBCO BRL).

Preferably, the expression control sequences will be eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the invention may follow; see, e.g., the appended examples.

An alternative expression system, which can be used to express a cell cycle interacting protein is an insect system. In one such system, Autographa californica nuclear polyhedrosis virus (AcNPV) is used as a vector to express foreign genes in Spodoptera frugiperda cells or in Trichoplusia larvae. The coding sequence of a recited nucleic acid molecule may be cloned into a nonessential region of the virus, such as the polyhedrin gene, and placed under control of the polyhedrin promoter. Successful insertion of said coding sequence will render the polyhedrin gene inactive and produce recombinant virus lacking coat protein coat. The recombinant viruses are then used to infect S. frugiperda cells or Trichoplusia larvae in which the protein of the invention is expressed (Smith, J. Virol. 46 (1983), 584; Engelhard, Proc. Nat. Acad. Sci. USA 91 (1994), 3224-3227).

Additional regulatory elements may include transcriptional as well as translational enhancers. Advantageously, the above-described vectors of the invention comprise a selectable and/or scorable marker.

Selectable marker genes useful for the selection of transformed cells and, e.g., plant tissue and plants are well known to those skilled in the art and comprise, for example, antimetabolite resistance as the basis of selection for dhfr, which confers resistance to methotrexate (Reiss, Plant Physiol. (Life Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule can be used alone or as part of a vector to express the polypeptide of the invention in cells, for, e.g., purification but also for gene therapy purposes. The nucleic acid molecules or vectors containing the DNA sequence(s) encoding any one of the above described polypeptide of the invention is introduced into the cells which in turn produce the polypeptide of interest. Gene therapy, which is based on introducing therapeutic genes into cells by ex-vivo or in-vivo techniques is one of the most important applications of gene transfer. Suitable vectors, methods or gene-delivery systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91 (1998), 30-36; Verma, Gene Ther. 5 (1998), 692-699; Nabel, Ann N.Y. Acad. Sci. 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; U.S. Pat. No. 5,589,466; or Schaper, Current Opinion in Biotechnology 7 (1996), 635-640. The recited nucleic acid molecules and vectors may be designed for direct introduction or for introduction via liposomes, or viral vectors (e.g., adenoviral, retroviral) into the cell. Preferably, said cell is a germ line cell, embryonic cell, or egg cell or derived there from, most preferably said cell is a stem cell. An example for an embryonic stem cell can be, inter alia, a stem cell as described in Nagy, Proc. Natl. Acad. Sci. USA 90 (1993), 8424-8428.

The invention also provides for a host transformed or transfected with a vector of the invention. Said host may be produced by introducing the above described vector of the invention or the above described nucleic acid molecule of the invention into the host. The presence of at least one vector or at least one nucleic acid molecule in the host may mediate the expression of a gene encoding the above described SLC38A9.

The described nucleic acid molecule or vector of the invention, which is introduced in the host may either integrate into the genome of the host or it may be maintained extrachromosomally.

The host can be any prokaryote or eukaryotic cell.

The term “prokaryote” is meant to include all bacteria, which can be transformed or transfected with DNA or RNA molecules for the expression of a protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the protein encoded by the polynucleotide of the present invention may be glycosylated or may be non-glycosylated. Especially preferred is the use of a plasmid or a virus containing the coding sequence of the polypeptide of the invention and genetically fused thereto an N-terminal FLAG-tag and/or C-terminal His-tag. Preferably, the length of said FLAG-tag is about 4 to 8 amino acids, most preferably 8 amino acids. An above described polynucleotide can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, loc cit.). Preferably, said the host is a bacterium or an insect, fungal, plant or animal cell.

It is particularly envisaged that the recited host may be a mammalian cell. Particularly preferred host cells comprise CHO cells, COS cells, myeloma cell lines like SP2/0 or NS/0. As illustrated in the appended examples, particularly preferred are CHO-cells as hosts.

More preferably said host cell is a human cell or human cell line, e.g. per.c6 (Kroos, Biotechnol. Prog., 2003, 19:163-168).

Herein provided is also a process for the production of a polypeptide to be used in accordance with the present invention, said process comprising culturing a host of the invention under conditions allowing the expression of the polypeptide of the invention and recovering the produced polypeptide from the culture.

The transformed hosts can be grown in fermentors and cultured according to techniques known in the art to achieve optimal cell growth. The polypeptide of the invention can then be isolated from the growth medium, cellular lysates, or cellular membrane fractions. The isolation and purification of the, e.g., microbially expressed polypeptides of the invention may be by any conventional means such as, for example, preparative chromatographic separations and immunological separations such as those involving the use of monoclonal or polyclonal antibodies directed, e.g., against a tag of the polypeptide of the invention or as described in the appended examples.

The conditions for the culturing of a host, which allow the expression are known in the art to depend on the host system and the expression system/vector used in such process. The parameters to be modified in order to achieve conditions allowing the expression of a recombinant polypeptide are known in the art. Thus, suitable conditions can be determined by the person skilled in the art in the absence of further inventive input.

Once expressed, the polypeptide of the invention can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure polypeptides of at least about 90 to 95% homogeneity are preferred, and 98 to 99% or more homogeneity are most preferred, for pharmaceutical uses. Once purified, partially or to homogeneity as desired, the polypeptide of the invention may then be used therapeutically (including extracorporeally) or in developing and performing assay procedures. Furthermore, examples for methods for the recovery of the polypeptide of the invention from a culture are described in detail in the appended examples.

Furthermore, the present invention provides a kit useful for carrying out the methods of the invention, the kit comprising a nucleic acid or an antibody capable of detecting the presence of SLC38A9 (like SLC38A9 mRNA or protein) as defined herein. Also envisaged herein is the use of the herein described kit for carrying out the herein provided methods.

For example, said kit may comprise (a) compound(s) required for specifically determining the presence of SLC38A9 (like SLC38A9 mRNA or protein) as defined herein. Moreover, the present invention also relates to the use of (a) compound(s) required for specifically determining the presence of SLC38A9 (like SLC38A9 mRNA or protein) as defined herein for the preparation of a kit for carrying out the methods of this invention. On the basis of the teaching of this invention, the skilled person knows which compound(s) is (are) required for specifically determining the presence of SLC38A9 (like SLC38A9 mRNA or protein) as defined herein. For example, such compound(s) may be (a) “binding molecule(s)”, like, for example, (a) antibody. Particularly, such compound(s) may be (a) (nucleotide) probe(s), (a) primer(s) (pair(s)), (an) antibody(ies) and/or (an) aptamer(s) specific for SLC38A9 (like SLC38A9 mRNA or protein) as defined herein. Such compounds may also be useful for determining the activity of SLC38A9 (like SLC38A9 mRNA or, primarily SLC38A9 protein) as defined herein. The kit (to be prepared in context) of this invention may be a diagnostic kit and in particular a kit for assaying candidate molecules.

The kit (to be prepared in context) of this invention may further comprise or be provided with (an) instruction manual(s). For example, said instruction manual(s) may guide the skilled person (how) to determine the presence of SLC38A9 as defined herein and/or (how) to determine the activity of SLC38A9 as defined herein. Said instruction manual(s) may comprise guidance to use or apply the herein provided methods or uses. The kit (to be prepared in context) of this invention may further comprise substances/chemicals and/or equipment suitable/required for carrying out the methods and uses of this invention. For example, such substances/chemicals and/or equipment are solvents, diluents and/or buffers for stabilizing and/or storing (a) compound(s) required for specifically determining the presence/activity of SLC38A9 as defined herein.

An exemplary antibody specifically binding to SLC38A9 that can be used in these kits and screening methods is commercially available from Sigma (HPA043785)).

Polyclonal or monoclonal antibodies or other antibodies (derived therefrom) for use in the kits or screening methods provided herein can be routinely prepared using, inter alia, standard immunization protocols; see Ed Harlow, David Lane, (December 1988), Antibodies: A Laboratory Manual, Cold Spring Harbor Laboratory; or Ed Harlow, David Lane, (December 1998), Portable Protocols (Using Antibodies): A Laboratory Manual 2^{nd} edition, Cold Spring Harbor Laboratory.

For example, the herein provided SLC38A9 proteins can be used as an antigen in the production of antagonizing antibodies. Inter alia, a protein or polypeptide can be used as antigen, wherein the protein/polypeptide consists of 15 to 25 contiguous amino acids of a SLC38A9 protein as defined herein (e.g. as shown in SEQ ID NO: 3 or related sequences). In particular, the protein/polypeptide can consist of 15 to 25 contiguous amino acids of the N-terminal cytoplasmic region of a SLC38A9 protein as defined and provided herein.

Preferably, a fragment of the protein shown in SEQ ID NO: 3 comprising or consisting of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids of the protein as shown in SEQ ID NO: 3 can be used as an antigen. As mentioned, particularly preferred are fragments of the protein shown in SEQ ID NO: 3 comprising or consisting of 15 to 25 contiguous amino acids of the protein as shown in SEQ ID NO: 3. Accordingly, a fragment of the protein shown in SEQ ID NO: 3 comprising or consisting of 15, 16, 17, 18, 19, 20, 21, 22, 23, 24, or 25 contiguous amino acids of the protein as shown in SEQ ID NO: 3 is provided and used herein.

The SLC38A9 protein can be produced recombinantly (i.e. produced in appropriate host cells) or synthetic (i.e. chemically synthesized). Recombinant production of SLC38A9 protein is described herein. For example, recombinant production can be achieved using any one of the molecular cloning and recombinant expression techniques known in the art. For example, a nucleic acid molecule encoding SLC38A9 protein can be introduced into an appropriate host cell, such as a bacterium, a yeast cell (e.g., a Pichia cell), an insect cell or a mammalian cell (e.g., CHO cell). The encoding nucleic acid molecule can be placed in an operable linkage to a promoter capable of effecting the expression of the SLC38A9 protein antigen in the host cell. SLC38A9 protein, which is expressed by the host cell, can be readily purified using routine protein purification techniques.

For example, the nucleotide sequence as set forth in SEQ ID NO: 1 or 2 or a nucleic acid sequence encoding the SLC38A9 shown in SEQ ID NO: 3 or encoding a fragment thereof, such as a protein consisting of 15 to 25 contiguous amino acids of the protein shown in SEQ ID NO: 3, can be cloned in an expression vector and placed in an operable linkage to a temperature sensitive promoter. The expression vector can be introduced into Escherichia coli and the antigen can be expressed upon heat induction. The cells can be lysed and the inclusion bodies where the antigen accumulates are separated by centrifugation. The recombinant protein in the inclusion bodies is solubilized using SDS or other solubilization agents known in the art such as urea, guanidine hydrochloride, sodium cholate, taurocholate, and sodium deoxycholate. In accordance with the present invention, a purified recombinant SLC38A9 protein can be combined with a pharmaceutically acceptable carrier to optimize its use as an antigen.

For example, immunization may involve the intraperitoneal or subcutaneous administration of the SLC38A9 protein/polypeptide (and/or fragments, isoforms, homologues and so on) as defined herein to a mammal (e.g. rodents such as mice, rats, hamsters and the like). Preferably, fragments of the SLC38A9 protein/polypeptide are used, wherein the fragment preferably bears the N-terminal cytoplasmic region of the SLC38A9 protein/polypeptide (or a fragment thereof) as defined herein.

Methods for the preparation and screening of antibodies that specifically bind to or specifically recognize a target protein/polypeptide are known in the art. For example, antibodies recognizing the SLC38A9 protein/polypeptide may be affinity purified. ELISA is commonly used for screening sera and/or assaying affinity column fractions. Western Blots can be used to demonstrate that the antibody can detect the SLC38A9 protein/polypeptide and to evaluate whether the antibody only recognizes the SLC38A9 protein/polypeptide, or if it cross-reacts with other proteins.

A person skilled in the art is in the position to apply and to adapt the teaching of these documents for the generation and validation of antibodies specifically binding to or specifically recognizing the SLC38A9 protein/polypeptide as defined herein in context of the present invention.

Herein provided is a method that uses SLC38A9 ((like the information associated with) the sequence encoding the protein or the amino acid sequence of the protein as defined herein) to derive pharmacological agents antagonising its activity and useful for treating, preventing or ameliorating a disease associated with mTORC1 activation comprising the administration of an antagonist of SLC38A9 to a subject in need of such a treatment, prevention or amelioration.

Herein provided is a method for treating, preventing or ameliorating a disease associated with mTORC1 activation comprising antagonizing/inhibiting (the activity of) SLC38A9 to a subject in need of such a treatment, prevention or amelioration.

All definitions provided herein above inter alia in relation to “SLC38A9”, “antagonists of SLC38A9” and “disease associated with mTORC1 activation”, “pharmaceutical compositions” and the like, apply mutatis mutandis here.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of.”

Thus, the terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means that specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

As used herein the term “about” refers to ±10%.

The present invention is further described by reference to the following non-limiting figures and examples. Unless otherwise indicated, established methods of recombinant gene technology were used as described, for example, in Sambrook, Russell “Molecular Cloning, A Laboratory Manual”, Cold Spring Harbor Laboratory, N.Y. (2001)) which is incorporated herein by reference in its entirety.

The Figures show:

FIG. 1:

a, Interactors of SLC38A9 identified by TAP-LC-MS/MS using GFP TAP as negative control. In addition proteins with a spectral count of 1 or a CRAPome³⁴ frequency of >0.1 were excluded. b and c, HEK293T cells were transfected with the indicated tagged constructs or empty vector (−). Anti-V5 immunoprecipitates (IP) and cell extracts (XT) were treated with PNGase (1 hour, 37° C.) and analysed by immunoblot. <: ST-HA-SLC38A9; *: non-specific band d, Lysates from control (empty vector) or shSLC38A9 transduced HEK293T cells were subjected to immunoprecipitation with the indicated antibodies, treated with PNGase and analysed by immunoblot e, Lysates from shGFP or shSLC38A9 transduced HeLa cells were subjected to immunoprecipitation with the indicated antibodies, treated with PNGase and analysed by immunoblot f, Lysates from K562 cells were subjected to immunoprecipitation with the indicated antibodies, treated with PNGase and analysed by immunoblot g, Tabular view of SLC38A9, SLC38A1, SLC38A2 and SLC36A1 TAP-LC-MS/MS analysis. Baits and interactors of SLC38A9 are shown with spectral counts (left, maximum of biological replicate) and sequence coverage (right, percentage). Empty box: not detected. h, HEK293T cells were transfected with the indicated tagged constructs or empty vector (−). Anti-HA immunoprecipitates (IP) and cell extracts (XT) were treated with PNGase (1 hour, 37° C.) and analysed by immunoblot i, Single-channel and merged confocal microscopy images of DAPI stained nuclei and indirect immunofluorescence against HA-tagged SLC38A9 and endogenous lysosomal markers LAMP1 (top panel) and LAMP2 (middle panel) and the non-induced and secondary-antibody only control (bottom panel) in HEK293 Flp-In TREx cells. Scale bar, 10 μm. Intensity profiles for SLC38A9 (white) and LAMP1, LAMP2 or secondary antibody control (black) along the cross-section lines indicated in the respective merged channel images are shown. Grey indicates signal overlap j, Quantification of HA-SLC38A9 signal above background (dashed lines in i) that colocalizes with LAMP1, LAMP2 or secondary-antibody only positive areas. Average and s.d. of at least two images is shown, analysing colocalization in 22, 34 and 27 cells respectively.

FIG. 2:

a, Interactors of LAMTOR1, LAMTOR3, LAMTOR4, LAMTOR5, RAGA and RAGC were identified by TAP-LC-MS/MS using GFP TAP as negative control. Proteins filtered using SAINT³⁵ that interacted with at least five bait proteins were retained. Previously published interactors of the Ragulator complex and RAG GTPases that were also detected are highlighted. b, Tabular view of spectral counts (maximum of biological replicate) and sequence coverage (percentage) of the core Ragulator network and published interactors detected. c HEK293T cells were transfected with the indicated constructs or empty vector (−). Anti-HA immunoprecipitates and cell extracts were analysed by immunoblot with the indicated antibodies. d and e, HEK293T cells were transfected with the indicated tagged SLC38A9 constructs or empty vector (−). Anti-HA immunoprecipitates and cell extracts were analysed by immunoblot with the indicated antibodies. Deletion constructs are labelled with the number of the encoded amino acids (d) and point mutants with the amino acid motif substituted to alanine (e).

FIG. 3:

a, Orientation of SLC38A9 in proteoliposomes. After purification on Ni-chelating chromatography His-SLC38A9 was incubated overnight at 37° C. in absence or in presence of 1 U thrombin. Immunoblot using anti-His or anti-SLC38A9 antibody. b, Proteoliposomes reconstituted with SLC38A9 were centrifuged, resuspended in isosmotic buffer, incubated overnight at 37° C. with 1 U thrombin, solubilized with SDS Immunoblot using anti-His or anti-SLC38A9 antibody. c, Time course of glutamine uptake by SLC38A9 in proteoliposomes reconstituted with purified SLC38A9 or with control empty vector protein. The uptake of 10 μM [³H]-glutamine was measured at different time intervals. Transport was stopped as described in Materials and Methods. Values represent means of specific transport ±s.d. from 8 different experiments. Significance was estimated by Student's t test (*P<0.01 or **P<0.001). d, Inhibition of the [³H]-glutamine uptake in proteoliposomes. The indicated amino acids were added together with 10 μM [³H]-glutamine Transport was measured at 60 min Values represent means of percent residual activity with respect to control (without added inhibitor) ±s.d. from 3 independent experiments. Significance was estimated by Student's t test (*P<0.01 or **P<0.001). e, Time course of glutamine efflux from proteoliposomes reconstituted with purified SLC38A9. Proteoliposomes were incubated with external 10 μM [³H]-glutamine After 120 min the residual external radioactivity was removed by gel-filtration and the time course of [³H]-glutamine efflux was measured as for the uptake (see FIG. 3 c). Values represent means of specific transport ±s.d. from 3 different experiments. f and g. HEK293T cells were transfected with the indicated tagged RAGA (0 or RAGB (g) and RAGC constructs or empty vector (−). Anti-HA immunoprecipitates and cell extracts were treated with PNGase and analysed by immunoblot with the indicated antibodies. Mutations are the following: RAGA: Q66L and T21N; RAGB: Q99L and T54N; RAGC: S75N and Q120N

FIG. 4:

a and b, HEK293T cells transduced with lentivirus-encoded shRNA against SLC38A9 or GFP were starved for 50 min in medium without amino acids and serum and then stimulated with amino acids (a) or cycloheximide (b, 25 μg/ml) for 10 or 20 min Cell lysates were analysed by immunoblot with antibodies against phosphorylated threonine 389 S6 kinase, phosphorylated S6, phosphorylated ULK1 and total S6 kinase. c, HEK293T were transfected with the indicated siRNA. After 72 h, cells were treated as in a.d, HeLa were transfected with the indicated siRNA. After 72 h, cells were starved for 50 min in medium without amino acids and serum and then stimulated with amino acids and insulin (1 uM). Cell lysates were analysed by immunoblot with antibodies against phosphorylated threonine 389 S6 kinase, phosphorylated S6 and total S6 kinase e, Model of Sentor/SLC38A9 as part of the lysosomal amino acid-sensing machinery

FIG. 5:

a, Table of SLCs belonging to amino acid transporter families robustly expressed in HEK293 and K562 cells as monitored by RNAseq. SLC members of amino acid transporter-containing families¹⁶ and expressed (FPKM>0.5) in both cell lines were ranked according to their expression level. The number of PubMed entries was obtained by querying the GeneSymbol. b, Expression of members of the SLC32, SLC36 and SLC38 families in HEK293 and K562 cells. c, Cell size measurements of HEK293T cells after short-hairpin (shRNA) mediated knockdown against GFP (control, dashed line) or SLC38A9 (solid line), measured by automated microscopy and image analysis. Sparse and interphase cells were selected using image analysis and machine learning, and nucleus diameter (μm) was used as robust proxy for cell size³³. Smoothed distributions of 2400 and 4165 cells, respectively, are shown. d, Cell proliferation measurement of HEK293T cells transduced with lentivirus-encoded shRNA against SLC38A9 or GFP. 10⁵ cells were seeded and counted every 24 h. Mean values ±s.d. from triplicates e. HEK293 Flp-In TREx cells inducibly expressing SLC38A9 were treated or not with doxycycline (Dox) for 24 h. Where indicated, cell lysates were treated with PNGase (1 hour, 37° C.) and analysed by immunoblot (IB)

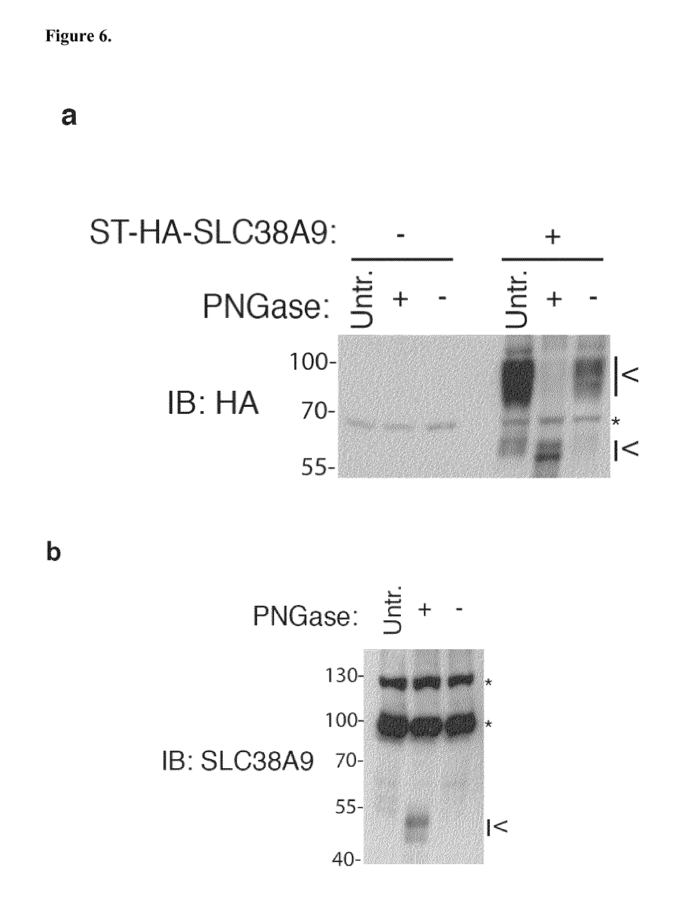

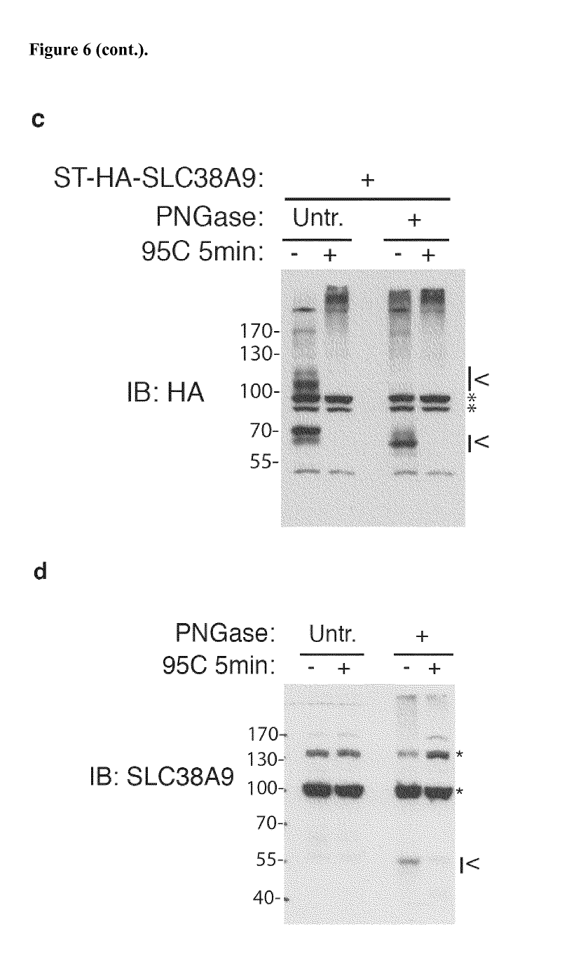

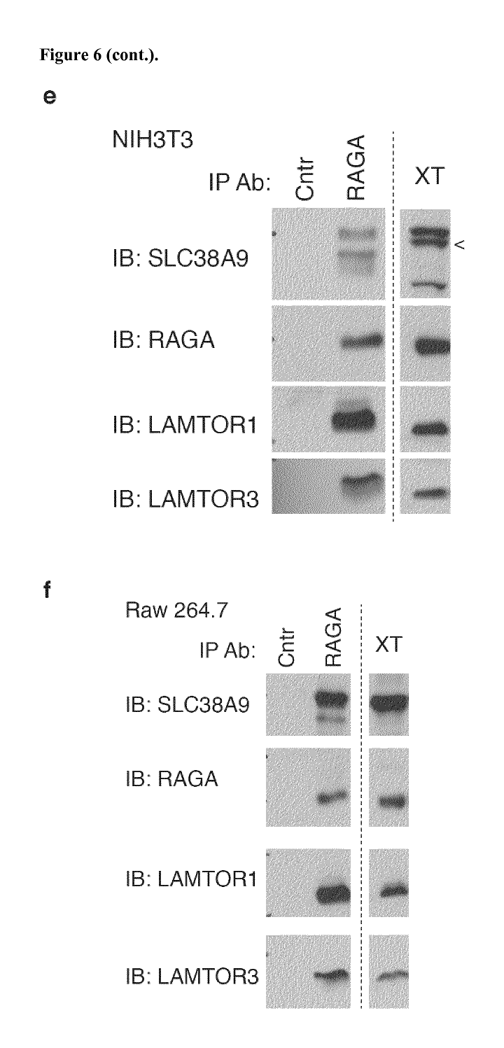

FIG. 6:

a-d, Where indicated, HEK293T cells were transfected with the tagged SLC38A9 constructs (+) or empty vector (a, c). Cell lysates were prepared and left untreated (Untr.) or incubated 1 hour at 37° C. in presence or absence of PNGase and analysed by immunoblot, <: SLC38A9; *: non-specific band. c and d depict the effect of boiling the lysates for 5 min at 95° C. after PNGase treatment. e and f Lysates from murine NIH3T3 (e) or Raw 264.7 (f) cells were subjected to immunoprecipitation with the indicated antibodies, treated with PNGase and analysed by immunoblot.

FIG. 7:

HEK293T cells were transfected with a streptavidin-HA tagged SLC38A9 construct. Single-channel and merged confocal microscopy images of DAPI stained nuclei and indirect immunofluorescence of HA-tagged SLC38A9 and endogenous lysosomal marker LAMP1. Scale bar, 10 μm.

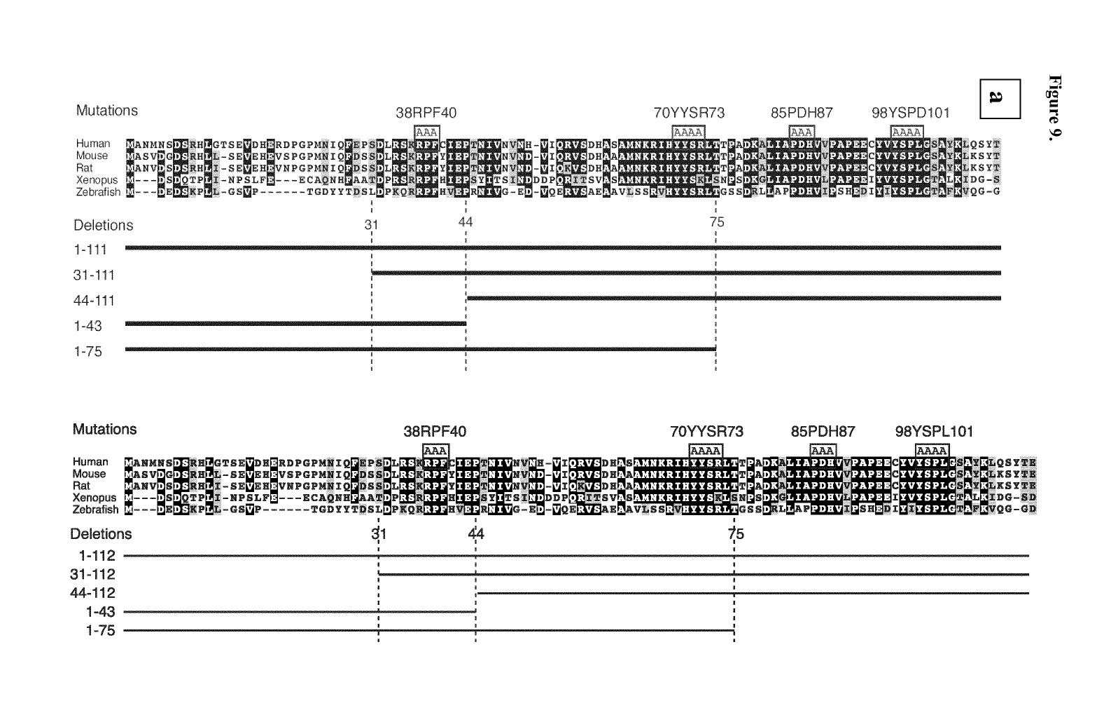

FIG. 8:

a-b, SLC38A9 peptides detected in LAMTOR1, 3, 4 and 5 (a) or in SLC38A9 (b) TAP-MS/MS analysis are mapped on SLC38A9 sequence and highlighted in bold. Transmembrane helices are underlined. Potential tryptic cleavage sites are in lower case.

FIG. 9:

a, Sequence alignment of the N-terminal cytoplasmic region (amino acids 1-111, and 1-112, respectively) of human, mouse, rat, Xenopus and zebrafish SLC38A9 Amino acids selected for deletion in FIG. 2c and motifs substituted to alanine in FIG. 2d-e are highlighted. Black and grey shading indicates >60% amino acid sequence identity and similarity, respectively. b and c HEK293T cells were transfected with the indicated tagged SLC38A9 constructs or empty vector (−). Anti-HA immunoprecipitates and cell extracts were analysed by immunoblot with the indicated antibodies. Deletion constructs are labelled with the number of the encoded amino acids (b) and point mutants with the amino acid motif substituted to alanine (c).

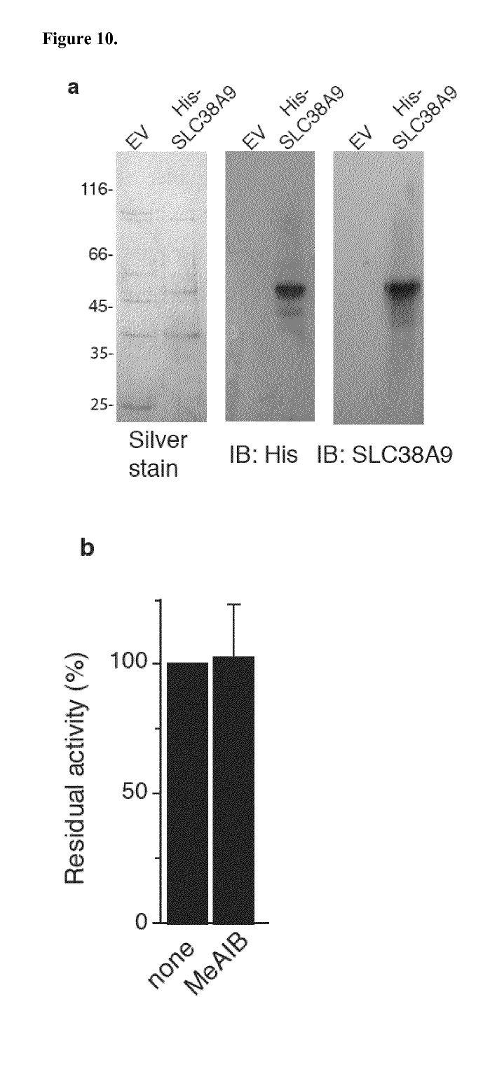

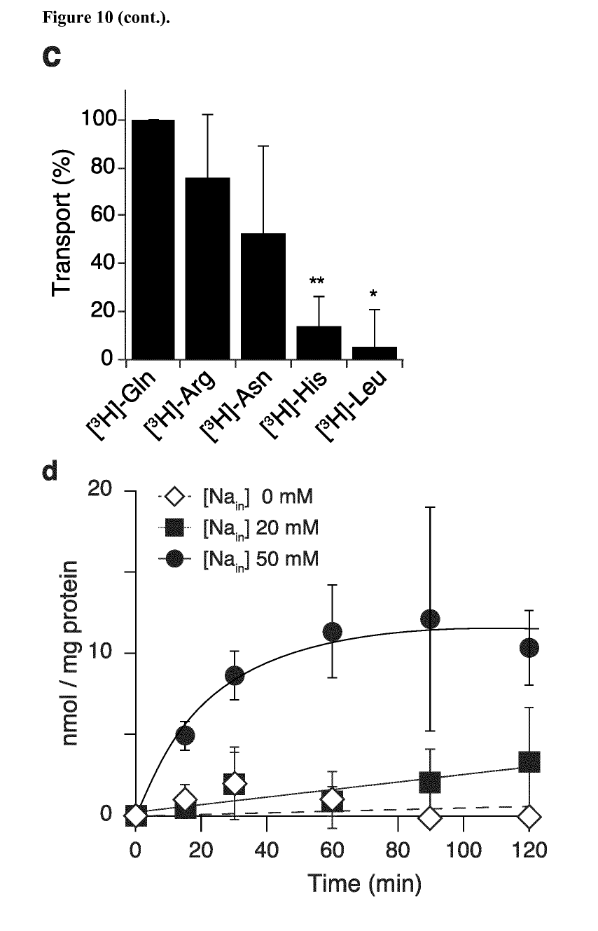

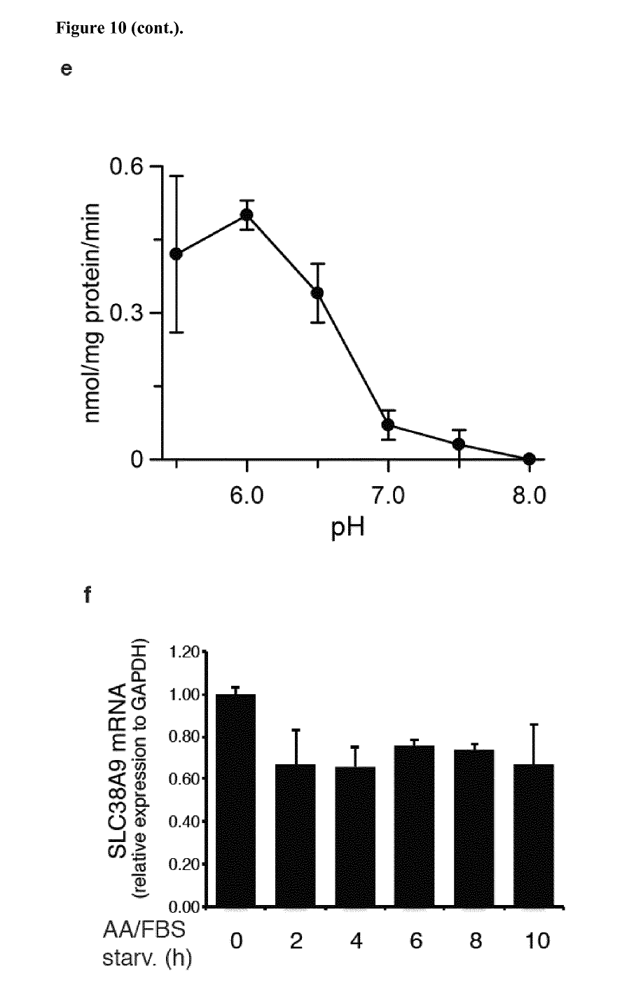

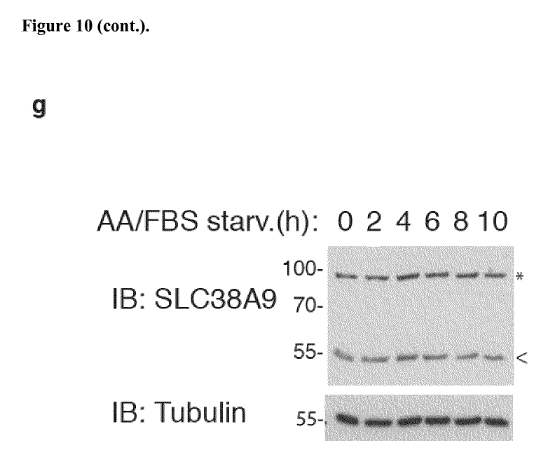

FIG. 10:

a, Purification of SLC38A9. Lanes represent empty vector control and SLC38A9 expressed in E. coli and purified on Ni-chelating chromatography Immunoblot of the same fractions using anti-His antibody or anti-SLC38A9 are shown. b, Inhibition of the [³H]-glutamine uptake in proteoliposomes. 1 mM MeAIB (α-(methylamino)isobutyric acid) was added together with 10 μM [³H]-glutamine Transport was measured at 60 min. Values represent means of percent residual activity with respect to control (without added inhibitor) ±s.d. from 3 independent experiments. Significance was estimated by Student's t test (*P<0.01 or **P<0.001). c, Uptake of the indicated [3H]-labelled amino acids (10 μM) by SLC38A9 in proteoliposomes measured at 60 min. Values represent means of percent in respect to glutamine transport measured in the same experiment ±s.d. from three independent experiments. Significance was estimated by Student's t test (*P<0.01 or **P<0.001). d, Time course of glutamine uptake by SLC38A9 in proteoliposomes reconstituted with the purified protein fraction. The uptake of 10 μM [³H]-glutamine was measured at different time intervals, in the presence of the indicated intraliposomal sodium concentrations. Transport was stopped as described in Materials and Methods and calculated by subtracting the radioactivity associated to proteoliposomes reconstituted with the empty vector fraction. Values represent means of specific transport ±s.d. from 3 different experiments. e, Effect of pH on the reconstituted SLC38A9. Reconstitution was performed as described in Materials and Methods except that 20 mM Hepes/Tris at the indicated pH was used. Transport was started by adding 10 μM [³H]-glutamine in 20 mM Hepes/Tris buffer at the indicated pH to proteoliposomes and stopped at 30 min. Results are means of specific transport rate ±s.d. from 3 different experiments f-g HEK293T cells were starved in medium without amino acids and serum for the indicated times. SLC38A9 expression was analysed by quantitative PCR (f) and immunoblot (g). <: SLC38A9; *: non-specific band

The Example illustrates the invention.

### EXAMPLE

### Sentor (SLC38A9) is an Integral Component of the Lysosomal Amino Acid-Sensing Machinery that Controls mTORC1 Activity and Antagonists of Sentor (SLC38A9) are Useful in the Therapy of Diseases Associated with mTORC1 Activity

### Material and Methods

### Antibodies

Antibodies used were SLC38A9 (HPA043785 Sigma), LAMTOR1 (8975 Cell Signaling), LAMTOR3 (8169 Cell Signaling), RAGA (4357 Cell Signaling), RAGC (5466 Cell Signaling), phospho-p70 S6 Kinase (Thr389) (9234 Cell Signaling), phospho-S6 (Ser240/244) (2215 Cell Signaling), phosphor-ULK1 (Ser757) (6888 Cell Signaling), raptor (2280 Cell Signaling), ATP6V1B2 (ab73404 Abcam), ATP6V1A (GTX110815 GeneTex), mouse anti-rabbit IgG (conformation specific) (3678 Cell Signaling), LAMP1 (ab25630 Abcam), LAMP2 (sc-18822 Santa Cruz), p70 S6 kinase (sc-230 Santa Cruz), Tubulin (ab7291 Abcam), HA (H6533 Sigma, MMS-101P Covance or sc-805 Santa Cruz), V5 (ab9116 Abcam), His (A7058 Sigma) and secondary HRP-conjugated antibodies (Jackson ImmunoResearch).

### Plasmids

Expression constructs were generated by PCR amplification followed by Gateway cloning (Invitrogen) into pTRACER-CVS-GW or pTO-SII-HA-GW¹ with N-terminal tagging for SLC38A9, SLC38A1, SLC38A2, SLC38A7, SLC36A1, SLC36A4, RAGA, RAGB, RAGC and LAMTOR3 (human and mouse) and C-terminal tagging for LAMTOR1, 4 and 5. Point mutations were introduced by site-directed mutagenesis (Invivogen).

### Cells

HEK293 Flp-In TREx cells that allow doxycycline-dependent transgene expression were from Invitrogen. HEK293 Flp-In TREx, HEK293T, HeLa, K562, NIH3T3 and Raw264.7 cells were kept in RPMI medium (PAA Laboratories) supplemented with 10% (v/v) FBS (Invitrogen) and antibiotics (100 U/ml penicillin and 100 mg/ml streptomycin).

### Transfections, Cell Lysis, Deglycosylation, and Immunoprecipitations

Cells were transfected with Polyfect (Qiagen) and used for experiments after 24 hours. For lysis, cells were resuspended in Nonidet-40 lysis buffer (1% NP-40, 50 mM Hepes pH7.4, 250 mM NaCl, 5 mM EDTA, Halt phosphatase inhibitor cocktail (ThermoScientific), one tablet of EDTA-free protease inhibitor (Roche) per 50 ml) on ice for 5 min. Lysates were cleared by centrifugation in a microcentrifuge (13000 r.p.m., 10 min, 4 C). Proteins were quantified with BCA (Pierce). For immunoprecipitations, lysates were precleared on Sepharose6 beads (Sigma) (40 min. with rotation, 4° C.) and then incubated either with HA- or V5-coupled beads (3 h with rotation, 4° C.) or with primary antibody and protein G-sepharose (GE healthcare) (14 h with rotation, 4° C.). Beads were recovered and washed four times with lysis buffer before analysis by SDS-PAGE and immunoblotting. When required, a mouse anti-rabbit IgG (conformation specific) antibody was used for immunoblot and revealed with an anti-mouse HRP-conjugated antibody to avoid detection of immunoglobulin heavy chains. In case of detection of endogenous SLC38A9, samples were treated with PNGase (NEB, 250 U for 30 ul, 1 h, 37° C.) before SDS-PAGE.

### RNAi

For shRNA-mediated knockdown, shRNA-encoding pLKO.1 targeting SLC38A9 (ThermoFisher, TRCN0000151238) or GFP (ThermoFisher, RHS4459) were used. Lentiviruses were produced using second-generation packaging plasmids pMD2-VSVG and pCMV-R8.91. HEK293T cells were co-transfected with packaging plasmids and the shRNA-encoding plasmids. Cells were washed 16 h after transfection. Virus-containing supernatants were collected 24 h after washing, filtered and used for infection. After infection, HEK293T cells were selected with puromycin (4 μg/ml). For siRNA-mediated knockdown, HEK293T cells were transfected with HiPerfect (Qiagen) with 60 nM of siRNA pool. After 72 h cells were subjected to amino acid stimulation as described. ON-TARGETplus SMARTpool against SLC38A9 (L-007337-02-0005, Target sequences: ACACUGAAGGAUACGGUAA (SEQ ID NO: 77), GAUCCUGGACCUAUGAAUA (SEQ ID NO: 78), GAAGAGUGCUAUGUGUAUA (SEQ ID NO: 79), CAUGUCAUUCAGAGGGUUA (SEQ ID NO: 80)), LAMTOR1 (L-020916-02-0010, Target sequences: UCUCCAGGAUAGCUGCUUA (SEQ ID NO: 81), GGCUUAUACAGUACCCUAA (SEQ ID NO: 82), AAGUGAGGGUAGAACCUUU (SEQ ID NO: 83), GUUUGUCACCCUCGAUAAA (SEQ ID NO: 84)) and Non-targeting pool (D-001810-10-05) were from ThermoScientific.

### Proteomics

Hp-in HEK293 T-Rex cell lines inducibly expressing SII-HA-tagged SLC38A9, SLC38A1, SLC38A2, SLC36A1, RAGA, RAGC, GFP or LAMTOR complex subunits were generated as described². Tandem affinity STREP-HA purifications were performed as previously described³. In brief, cells were stimulated with doxycycline/tetracycline for 24 h to induce expression of SII-HA-tagged bait proteins. Induction of SII-HA-LAMTOR 3 was combined with 9 h starvation in serum free medium. Protein complexes were isolated by TAP using streptavidin agarose followed by elution with biotin, and a second purification step using HA-agarose beads. Proteins were eluted with 100 mM formic acid, neutralized with triethylammonium bicarbonate (TEAB) and digested with trypsin, and the peptides were analysed by LC-MS/MS.

### MS Data Analysis

Peak list data were extracted from RAW files using ProteoWizard (release 3.0.3201-http at proteowizard.sourceforge.net/) and searched against human SwissProt database version v2013.01_20130110 (37,261 sequences and common contaminants). The search engines MASCOT (v2.3.02, MatrixScience, London, UK) and Phenyx (v2.5.14, GeneBio, Geneva, Switzerland)⁴ were used. The searches were submitted to MASCOT using in-house perl scripts at precursor and fragment ions mass tolerances ±10 ppm and ±0.6 Da, respectively. Using the high-confidence identifications from this search, precursor and fragment ion masses were recalibrated for a second-pass search on MASCOT and Phenyx with precursor and fragment ions mass tolerances ±4 ppm and ±0.3 Da, respectively. One tryptic missed-cleavage was permitted. Carbamidomethyl cysteine and oxidized methionine were set as fixed and variable modifications, respectively. A false discovery rate of <0.25% and <0.1% were used for proteins and peptides, respectively, as described′. SAINT AP-MS filtering software was used to filter the interactions. All prey proteins with a SAINT AvgP of >0.95 were identified as high-confidence interactors.

### Immunofluorescence

HEK293T cells were plated on fibronectin-coated glass coverslips and, after 16 hours, induced with doxycycline or transfected. After 24 h, cells were washed with PBS, fixed (PBS, 4% formaldehyde) and permeabilized (PBS, 0.3% Saposin, 10% FBS). Slides were incubated with anti-HA (sc-805 Santa Cruz), anti-LAMP1 (ab25630 Abcam) or anti-LAMP2 (sc-18822 Santa Cruz) antibodies (1 hour, 25° C., PBS, 0.3% Saposin, 10% FBS). After three washes slides were incubated with goat anti-mouse AlexaFluor568 or anti-rabbit AlexaFlour488 antibodies (Invitrogen, 1 hour, 25° C., PBS, 0.3% Saposin, 10% FBS). After DAPI staining, slides were washed three times and mounted on coverslips with ProLong Gold (Invitrogen). Images were taken with a Zeiss Laser Scanning Microscope (LSM) 700. Images were exported from lsm files to tiff files, and analysed using custom Matlab code. Nuclei and cell outlines were detected based on the DAPI and combined immunofluorescence stains respectively, and colocalization measurements were restricted to cytoplasmic regions. Colocalization was measured as the percentage of SCL38A9 (green) pixel values above background that are also above background in the LAMP1 or LAMP2 (red) channel. The SLC38A9 and LAMP1 or LAMP2 colocalization was verified to be robust to variations in the background threshold, and also shows up as significant pixel value correlations between the red and green channels.

### Cell Size Measurements

HEK293T cells transduced with shRNA against SLC38A9 or GFP cells were seeded 24 h before fixation (PBS, 4% formaldehyde), permeabilized (PBS, 0.3% Saposin, 10% FBS) and stained with DAPI. Images were taken by automated microscopy using the PerkinElmer Operetta with 20× magnification in confocal mode. Images were analysed using CellProfiler (www at cellprofiler.org), CellClassifier (http at www.pelkmanslab.org/?page_id=63), Population Context measurement code (https at www.pelkmanslab.org/?page_id=1150) and custom Matlab code written specifically for this study. CellProfiler was used to detect individual nuclei on each image, and iterative machine learning using CellClassifier was applied to detect properly segmented interphase nuclei. Population context measurement code was used to measure the local cell density of each individual cell, and cell size measurements were restricted to sparse cells to avoid local crowding from confounding the measurements. We used the typical nucleus diameter (i.e. the diameter of a circle with the same area as that measured for each nucleus) as a robust proxy for cell size⁶. We confirmed that the cell size reduction induced by SLC38A9 shRNA treatment were present for a broad range of different local cell densities.

### Cell Proliferation Measurements

HEK293T cells transduced with shRNA against SLC38A9 or GFP were seeded and counted every 24 h using Casy (Roche).

### Amino Acids Starvation and Stimulation

HEK293T cells were washed with PBS and incubated in amino acid-free RPMI for 50 min Cell were then stimulated for 10 or 20 min by the addition of RPMI containing a two time concentrated solution of amino acids. After stimulation, the final concentration of amino acids in the media was the same as in RPMI. In case of cycloheximide treatment, amino acid-starved cells were stimulated by addition of cycloheximide diluted in amino acid-free RPMI at a final concentration of 25 μg/ml. HeLa cells were stimulated for 10 or 20 min by the addition of RPMI containing a two time concentrated solution of amino acids and insulin (1 uM final concentration, Sigma, 19278) Amino acid-free RPMI medium powder (R8999-04A, US biological) was complemented with sodium bicarbonate and sodium phosphate, dissolved in water, adjusted to pH7.4 and filtered. RPMI containing a two time concentrated solution of amino acids was obtained by complementing amino acid-free RPMI medium with RPMI 1640 amino acids solution (R7131, Sigma), adjusted to pH7.4 and filtered. L-glutamine (59202C, Sigma) was added shortly before usage.

### Q-PCR

RNA was isolated with RNeasy kit (Qiagen) and reverse-transcribed with oligo(dT) primers and a RevertAlD RT-PCR kit (Fermentas) and was analyzed by quantitative PCR. Primers:

SLC38A9_Fw:
(SEQ ID NO: 85)
TCCTTTGGGCAGTGGTCGAG
SLC38A9_Rev:
(SEQ ID NO: 86)
ACTCCCGGCACTTGGACAAA
GAPDH_Fw:
(SEQ ID NO: 87)
GAAGGTGAAGGTCGGAGT
GAPDH_Rev:
(SEQ ID NO: 88)
GAAGATGGTGATGGGATTTC

### Cloning, Expression and Purification of Recombinant Human SLC38A9

The human SLC38A9 cDNA was optimized according to E. coli codon usage by GenScript. In this optimized gene, the Codon Adaptation Index (CAI) was upgraded from 0.63 (wild type) to 0.87, the GC content and unfavourable peaks were optimized to prolong the half-life of the mRNA and a ribosome binding site was removed. The optimized cDNA was then sub-cloned cloned into expression vector (pH6EX3-His₆-hSLC38A9)⁷. The plasmid was used to transform E. coli Lemo21(DE3)pLysS (NEB). Selection on LB-agar was performed as previously described⁷. 0.1 mM rhamnose was added to modulate RNA polymerase expression. After addition of 0.4 mM IPTG cells were grown at 39° C. for 2 h. Cells were treated as previously described⁷. The protein patterns of the cell lysate fractions were analyzed by SDS-PAGE. The insoluble cell fraction (about 1.5 mg proteins) from cells expressing SLC38A9 or empty vector transfected cells, was washed with 100 mM Tris/HCl and resuspended in 100 mM (β-ME, 3.5 M urea, 0.5% sarkosyl, 200 mM NaCl, 10% glycerol, 20 mM Tris/HCl pH 8.0 and centrifuged at 12,000 g for 10 min. at 4° C. The resulting supernatant (about 1 mL) was applied onto a column (0.5 cm×2.5) filled with His select nickel affinity gel (Sigma) pre-conditioned with 8 mL of 0.1% sarkosyl, 200 mM NaCl, 10% glycerol, 10 mM Tris/HCl pH 8.0. The elution was performed with 10 mL of 0.1% C₁₂E₈, 150 mM NaCl, 10% glycerol, 5 mM DTE, 10 mM Tris/HCl pH 8.0 (washing buffer), 1.4 mL of the same buffer plus 10 mM imidazole; then the purified protein fraction (4-7 μg protein) was eluted by 1.4 mL of the same buffer plus 50 mM imidazole.

### Reconstitution of SLC38A9 in Proteoliposomes and Transport Measurements

The purified fractions from SLC38A9 or empty vector preparation were reconstituted by removing the detergent as previously described⁸ with a batch-wise procedure from a mixture of 400 μL of protein (about 2 μg protein in 0.1% C₁₂E₈, β-ME 6 mM, 10% glycerol, 20 mM Tris/HCl pH 8.0, 150 mM NaCl, 50 mM imidazole), 80 μL of 10% C₁₂E₈, 100 μL of 10% egg yolk phospholipids (w/v), 20 mM Hepes/Tris pH 6.5. 600 μL of proteoliposomes were passed through a Sephadex G-75 column (0.7 cm diameter×15 cm height) preequilibrated with 20 mM Hepes/Tris pH 6.5. Transport (uptake) measurement was started adding 10 μM [³H]glutamine or other radioactive substrates as indicated (0.5 μCi/nmol) to 100 μL proteoliposomes aliquots at 25° C. Transport was stopped by applying each sample of proteoliposomes on a Sephadex G-75 column (0.6×8 cm) to separate the external from the internal radioactivity. For efflux measurements, aliquots of the same pool of proteoliposomes passed through a Sephadex G-75 column (0.7 cm diameter×15 cm height) preequilibrated with 20 mM Hepes/Tris pH 6.5 were incubated with external 10 μM [³H]glutamine After 120 mM of loading, proteoliposomes were passed again through a Sephadex G-75 column (0.7 cm diameter×15 cm height) preequilibrated with 20 mM Hepes/Tris pH 6.5, for removing the residual external (not taken up) radioactivity. The time course of [³H]glutamine efflux was then measured stopping the efflux reaction at each time interval by applying proteoliposome samples on a Sephadex G-75 column (0.6×8 cm) to separate the external from the internal radioactivity. In both uptake and efflux assays, proteoliposomes eluted with 1 mL 50 mM NaCl were collected in scintillation cocktail for counting. The amount of reconstituted recombinant protein was estimated as previously described⁷. Time course data were interpolated by a first order rate equation from which the initial rate of transport was calculated as k x transport at equilibrium. L-Glutamine [3,4-³H(N)] from PerkinElmer; L-Histidine [ring-2,5-³H], L-Asparagine [³H] from Campro Scientific.

### Orientation of SLC38A9 in Proteoliposomes.

After purification as described in the section “Cloning, expression and purification of recombinant human SLC38A9”, His-SLC38A9 was incubated overnight at 37° C. in absence or in presence of 1 U thrombin (GE healthcare reconstituted according to the manufacturers in PBS 1× Invitrogen). After incubation the different samples were assayed by immunoblotting analysis using anti-His or anti-SLC38A9 antibody. This assay represents the control for the following orientation assay. To assess the orientation of SLC38A9, proteoliposomes (200 μl) reconstituted as described in the section “Reconstitution of SLC38A9 in proteoliposomes and transport measurements”, were centrifuged at 108.000×g for 90 minutes, resuspended in 20 mM Hepes/Tris pH 6.5, incubated overnight at 37° C. with 1 U thrombin in the same conditions of the purified protein. After incubation proteoliposomes were dissolved by 2.5% SDS and 0.2M Tris/HCl pH 6.8 immunoblotting analysis was performed as described for the purified protein.

### Assay of pH Dependence of the SLC38A9 Function.

Reconstitution was performed as described in the section “Reconstitution of SLC38A9 in proteoliposomes and transport measurements”, using buffer (Hepes/Tris) at different pH. Transport (uptake) was started as described in the section “Reconstitution of SLC38A9 in proteoliposomes and transport measurements”, by adding, to proteoliposomes, 10 μM [³H]-glutamine in 20 mM Hepes/Tris buffer at the same pH of the reconstitution mixture and stopped after 30 mM, as described in the same section.

### Analysis of Intraliposomal Sodium Dependence

SLC38A9 was purified as described in the section “Cloning, expression and purification of recombinant human SLC38A9” omitting NaCl from elution buffer (0.1% C₁₂E₈, 10% glycerol, 5 mM DTE, 10 mM Tris/HCl pH 8.0, plus imidazole 50 mM). Reconstitution was performed as described in section “Reconstitution of SLC38A9 in proteoliposomes and transport measurements”, in the absence or in the presence of 20 or 50 mM NaCl. Transport (uptake) measurement was performed as described in the same section.

### Results

Several members of the solute carrier (SLC) group belonging to families capable of transporting amino acids at the plasma membrane have been shown to regulate mTOR activity¹⁵. Thus, we assumed that other amino acid transporting SLCs might be candidates for the missing sensor of amino acid availability at the lysosome. As this is a fundamental process in human cells, we started by assuming ubiquitous presence and monitored expression levels of the members of SLC families competent for amino acid transport¹⁶ by RNAseq in two different cell lines (FIG. 5A). Among the list of robustly expressed SLCs, after exclusion of those characterized in numerous publications, we focused on member 9 of the SLC38 family as it was completely uncharacterized, showed vesicular staining¹⁷ and had been associated to lysosomes by proteomic analysis¹⁸.

The SLC38 (also known as sodium-coupled neutral amino acid transporter, SNAT) family counts eleven members, and is part of a major phylogenetic cluster of amino acid transporters comprising also the SLC32 and SLC36 families¹⁹′²⁰(FIG. 5B). SLC38A9 (UniProt: D6RDH2_HUMAN; NCBI NP_775785.2) is predicted to encompass eleven transmembrane helices and a 120-residue cytoplasmic N-terminal region. Overexpressed SLC38A9 was detected on SDS-PAGE mainly as a diffused band migrating higher than the expected size, suggesting postranslational modification, possibly glycosylation. Indeed treatment with peptide-N-glycosidase (PNGase) F resulted in the collapse of the different forms to faster migrating defined bands (FIG. 6A). Accordingly, endogenous SLC38A9 was detected only after deglycosylation (FIG. 6B).

We set out to test the possibility of an involvement of SLC38A9 in mTORC1 signalling. As mTORC1 is involved in cell growth′, we monitored cell size and cell proliferation after down-regulation of SLC38A9 by short hairpin RNA (shRNA) interference in HEK293T cells. Silencing of SLC38A9 resulted in a clear reduction of cell size and impairment in the ability of the targeted cells to proliferate, supporting a possible role of this protein in growth regulatory pathways and motivating further investigations (FIG. 5C-D).

If the SLC38A9 protein was indeed involved in the regulation of the mTORC1 complex, there should be a physical association with previously characterized members of the mTORC1 multiprotein complex. We engineered HEK293 cells to express tagged SLC38A9 upon doxycycline treatment and verified localisation to lysosomes (FIG. 1I, FIG. 5E, FIG. 7). We used this system to induce SLC38A9 expression and purify endogenously assembled protein complexes using tandem affinity purification (TAP) coupled to one-dimensional gel-free liquid chromatography tandem mass spectrometry (LC-MS/MS) and bioinformatic analysis. The choice of a gel-free approach was critical as we noticed that upon boiling SLC38A9 formed insoluble aggregates that impaired the ability of the protein to enter SDS-polyacrylamide gels (FIG. 6C-D). Remarkably, the analysis resulted in the identification of all the five members of the Ragulator complex (LAMTOR1-5) as well as all the four RAG GTPases as specific interactors of SLC38A9, while none of these proteins were identified in control purifications using GFP (FIGS. 1A and 1G). Such collective high sequence coverage of all components of the Ragulator/RAG GTPases complex strongly indicated that SLC38A9 was an additional uncharacterized component. We hypothesized that previous mass spectrometry-based characterizations may have missed this particular moiety because of the heat-induced aggregation combined with gel-based purification procedures (FIG. 6C-D).

When co-expressed in HEK293T cells, SLC38A9 co-immunoprecipitated with LAMTOR1 (FIG. 1B). Furthermore, overexpressed LAMTOR1 bound endogenous SLC38A9 (FIG. 1C). Importantly, we could validate complex membership entirely at the endogenous level in different human cell lines. With extract from HEK293T we used an antibody specific for SLC38A9 (commercially available from Sigma HPA043785) and detected coimmunoprecipitated RAGA and LAMTOR1 proteins (FIG. 1D). Conversely, immunoprecipitation of RAGA resulted in the specific recruitment of endogenous SLC38A9 (FIG. 1D). This association was not observed when SLC38A9 was previously silenced by shRNA confirming the specificity of the detected interactions. Association of endogenous SLC38A9 and RAGA was confirmed also in HeLa and K562 cells (FIGS. 1E and 1F). Furthermore, we could detect this complex in two murine cell lines, i.e NIH3T3 fibroblasts and Raw264.7 macrophage (FIGS. 6E and 6F).

To further challenge the specificity of these interactions we decided to investigate the protein complexes formed around the two highest expressed members of the SLC38 family, SLC38A1 and SLC38A2, as well as SLC36A1/PAT1, that has been previously associated to amino acid induced mTOR activation and the Ragulator/RAG GTPase complex and applied the identical proteomic strategy. Despite very high bait recovery, none of the member of Ragulator/RAG GTPase complex was identified among the interactors of these closely related transporters highlighting that the association of SLC38A9 with this complex is a unique property of this family member (FIG. 1G). To corroborate the proteomic analysis and further test specificity we immunoprecipitated SLC38A9, SLC38A1, SLC38A2, SLC36A1/PAT1 as well as a lysosomal member of the SLC38 family, SLC38A7, or the second member of the SLC36 family that has been shown to influence cell growth, SLC36A4/PAT4. Of these only SLC38A9 co-immunoprecipitated endogenous LAMTOR1, LAMTOR3, RAGA and RAGC, with both low and high bait expression levels (FIG. 1H). Thus, all available evidence was compatible with SLC38A9 being a lysosomal component the Ragulator/RAG GTPase complex.

The highest possible requirement for membership to this multiprotein complex would entail physical association with any of the several detected members in reciprocal purifications. We performed affinity purification coupled to mass spectrometry with LAMTOR1, 3, 4 and 5. We combined all independent purifications and reasoned that proteins that would be bound by all four proteins with robust sequence coverage would qualify as integral members of the complex′. Notably, at the core of the interacting network with all four baits we found all members of the Ragulator complex, the four RAG GTPases, RAPTOR and, satisfyingly, SLC38A9 (FIG. 2A-B). We further extended this proteomic analysis by determining the interactors of RAGA and RAGC: this resulted in the identification of SLC38A9, RAPTOR and all component of the Ragulator (FIG. 2A-B). The lower overall sequence coverage of SLC38A9 compared to other members of the complex could be ascribed to not accessible proteolytic cleavage of the transmembrane portions of the protein (FIG. 2B). Indeed the cytoplasmic N-terminus was sequenced with almost perfect efficiency and reflects the coverage obtained when SLC38A9 was used as bait (FIG. 8). Moreover we confirmed by immunoprecipitation the interaction of endogenous SLC38A9 with all baits used (FIG. 2C). The quality of the proteomic survey was also indicated by detection of the subunit VA0D1 of the V-ATPase complex and the FLCN-FNIP2 complex, a recently identified GAP for RAGC/D²³. Interestingly, we did not detect any other SLC member of the amino acid transporter family in any of the LAMTOR purifications, indicating that SLC38A9 is, at least in this cellular system, the only SLC interacting with the Ragulator complex.

Altogether these data established SLC38A9 as an integral part of the lysosomal amino acid-sensing machinery known to control mTOR activation.

To define the molecular basis of the interaction of SLC38A9 with the Ragulator/RAG GTPase complex we generated SLC38A9 deletion constructs encoding the N-terminal cytoplasmic tail (amino acids 1-111, or amino acids 1-112) or the remaining eleven transmembrane-containing regions (113-561). The cytoplasmic region of SLC38A9 retained the ability to interact with endogenous LAMTOR1, LAMTOR3, RAGA and RAGC proteins similar to the full-length protein, whereas binding was completely lost when the region was deleted (FIG. 2D). This indicated that the cytoplasmic tail, devoid of any transmembrane region, is required and sufficient to bind the Ragulator/RAG GTPases complex. Further deletion studies mapped the minimal binding region to amino acids 31-111, or to amino acids 31-112 (FIGS. 9A and B). Next, we identified four conserved motifs in the region (38RPF40, 70YYSR73, 85PDH87 and 98YSPL101) and individually mutated them to alanine (FIG. 9A). Disruption of any of the first three motifs completely abolished the binding ability of the cytoplasmic region of SLC38A9 towards LAMTOR1, LAMTOR3, RAGA and RAGC whereas mutation of the fourth motif had no effect (FIG. 2E). This observation was also confirmed in the context of full length SLC38A9 (FIG. 9C). Whereas the N-terminal cytoplasmic region is evolutionary conserved across SLC38A9 proteins, we could not detect any significant homology with the N-terminal region of any of the other SLC38 family members. These results defined the unique cytoplasmic region of SLC38A9 as responsible for the interaction with the lysosomal mTOR-activating machinery and indicated that evolutionary conserved motifs are required for this interaction to occur.

It has previously been shown that members of the SLC38 family of transporters prefer glutamine^{19,20,24} which is, together with leucine and arginine, the main amino acid involved in the regulation of mTORC1 activity^{6,12,15}. Therefore, we monitored the transporter activity of SLC38A9 towards this amino acid in a heterologous, biochemically defined system without confounding transporters or regulators.

We expressed SLC38A9 in E. coli using a codon-optimized tagged form, purified it and incubated with detergent and lipids to reconstitute proteoliposomes (FIG. 10A)²⁵. The orientation of the transporter in proteoliposomes corresponded to that observed in lysosomes (FIGS. 3A and B). Addition of [³H]-glutamine resulted in a time-dependent transport that was significant over the control proteoliposomes (FIG. 3C). Intraliposomal sodium was required for transport (FIG. 10D), but not addition of external sodium (not shown). Membrane potential artificially created by potassium gradients in the presence of valinomycin both positive outside or inside did not influence the transport activity of SLC38A9 (not shown). The study of the pH dependence of [3H]-glutamine uptake revealed that transport was more active at acidic pH range (pH 5.5-6.5) (FIG. 10E), according to the localization of the transporter in lysosomes and differently from the pH dependence described for SLC38A1 which is localized in the plasma membrane and is more active at alkaline pH range. Experiments showed that some polar amino acids were capable of competing efficiently for glutamine transport whereas inhibitor of system A SLC38 family member MeAIB had not effect (FIG. 3D, FIG. 10B). Direct transport assays further revealed SLC38A9 competence for [3H]arginine and [3H]asparagine, but not for [3H]leucine or [3H]histidine (FIG. 10C). The low ability of arginine to compete with glutamine transport, as previously reported also for SLC38A7, may reflect differences in binding and/or transport properties for the two amino acids. In term of transport efficiency, the initial rate calculated for 10 μM glutamine in the time course was 0.42±0.10 nmol per mg of protein per minute, which is moderate when compared to other reconstituted transporters′. Efflux of [3H]-glutamine from proteoliposomes was detected, with a calculated rate of 1.7±0.30 nmol/min/mg indicating that SLC38A9 is competent for bidirectional transport of amino acids (FIG. 3E). This suggests that SLC38A9 may be a low capacity transporter similar to SLC38A7²⁴ and resembling the properties of amino acid sensors described in yeast and Drosophila²⁷.

The ability of RAG GTPase to recruit mTOR by binding Raptor is critically dependent on their nucleotide loading status that is in turn regulated by amino acid availability. To test whether SLC38A9 interaction with RAG GTPase is dependent on their nucleotide loading we took advantage of widely used RAG nucleotide-binding mutants. RAGA Q66L, RAGB Q99L and RAGC Q120L mutation abolished GTPase activity and therefore are loaded with GTP, whereas RAGA T21N, RAGB T54N and RAGC S75N have much lower affinity for all nucleotide with preferential binding to GDT. By immunoprecipitating different combinations of mutants of RAGA or RAGB with RAGC we could recapitulate previous regulated interaction with RAPTOR and LAMTOR proteins (FIGS. 3F and G). Strikingly, SLC38A9 binding to RAG GTPase was dramatically different between the different RAG GTPases mutant pairs and show opposite behaviours than what observed for RAPTOR (FIGS. 3F and G). These results indicate that this interaction is regulated and dependent on RAG GTPase nucleotide loading status.

As expected for positive regulators of mTORC1 activity, silencing of SLC38A9 resulted in reduced cell size and proliferation. Thus, we investigated the functional relevance of SLC38A9 in amino acid sensing by monitoring mTORC1 activation in response to amino acids through the phosphorylation of its substrate ULK1 and S6 kinase²⁸ as well as its downstream target S6. Withdrawal of amino acids results in a rapid inactivation of mTORC1 that can be reverted by amino acid readdition. Suppression of SLC38A9 expression in HEK293T by shRNA resulted in a strong reduction of amino acid-induced mTORC1 activation (FIG. 4A). Similar results were obtained when SLC38A9 expression was reduced by small interfering RNA (siRNA): silencing of SLC38A9 suppressed amino acid-induced mTORC1 activation with similar efficiency as silencing of the positive control Lamtor1 (FIG. 4C). Moreover, we confirmed the role SLC38A9 in HeLa cells where knockdown of SLC38A9 expression blunted mTORC1 activity after amino acid and insulin stimulation (FIG. 4D). Cycloheximide has been shown to mimic amino acids stimulation by blocking protein synthesis and thus inducing accumulation of intracellular amino acids^{4,29,30}. Depletion of SLC38A9 also impaired cycloheximide-induced mTORC1 activation (FIG. 4B). This suggested that SLC38A9 participated to mTORC1 activation at the lysosome rather than contributing to the import of extracellular amino acid at the plasma membrane. Moreover, in contrast to several SLCs responsible for importing amino acids at the plasma membrane, including SLC38A2³¹, that are induced upon amino acid starvation, SLC38A9 mRNA or protein levels did not appear to be regulated by amino acid starvation (FIG. 10E-G).

Altogether the work presented here identifies SLC38A9 as a novel integral and druggable³² component of the lysosomal machinery that controls mTORC1 activity in response to amino acids (FIG. 4E). SLC38A9 displays the characteristics expected for the missing lysosomal amino acid sensor required for activation of mTOR and we therefore name it herein Sentor, for sensor of mTOR.

The present invention refers to the following nucleotide and amino acid sequences:

The sequences provided herein are available in the NCBI database and can be retrieved from www at ncbi.nlm.nih.gov/sites/entrez?db=gene; Theses sequences also relate to annotated and modified sequences. The present invention also provides techniques and methods wherein homologous sequences, and variants of the concise sequences provided herein are used. Preferably, such “variants” are genetic variants.

### SEQ ID No. 1:

Nucleotide sequence encoding homo sapiens SLC38A9 (Sentor) (NCBI geneID: 153129; Isoform1: NM_173514.3->NP_775785.2, complete sequence)

### SEQ ID No. 2:

Nucleotide sequence encoding homo sapiens SLC38A9 (Sentor) (NCBI geneID: 153129; Isoform1: NM_173514.3->NP_775785.2, open reading frame):

### SEQ ID No. 3:

Amino acid sequence of homo sapiens SLC38A9 isoform1 (NP_775785.2):

### SEQ ID No. 4:

Nucleotide sequence encoding homo sapiens SLC38A9 (Sentor) (NCBI geneID: 153129; Isoform1: NM_173514.3->NP_775785.2, open reading frame, mRNA):

### SEQ ID No. 5:

Nucleotide sequence of siRNA 1 (antisense sequence) targeting nucleotide sequence encoding homo sapiens SLC38A9 (Sentor)

compl-rev:
UUACCGUAUCCUUCAGUGU

### SEQ ID No. 6:

Nucleotide sequence of siRNA 2 (antisense sequence) targeting nucleotide sequence encoding homo sapiens SLC38A9 (Sentor)

compl-rev:
UAUUCAUAGGUCCAGGAUC

### SEQ ID No. 7:

Nucleotide sequence of siRNA 3 (antisense sequence) targeting nucleotide sequence encoding homo sapiens SLC38A9 (Sentor)

compl-rev:
UAUACACAUAGCACUCUUC

### SEQ ID No. 8:

Nucleotide sequence of siRNA 4 (antisense sequence) targeting nucleotide sequence encoding homo sapiens SLC38A9 (Sentor)

compl-rev:
UAACCCUCUGAAUGACAUG

### SEQ ID No. 9:

Nucleotide sequence of shRNA targeting nucleotide sequence encoding homo sapiens SLC38A9 (Sentor)

CCGGGCCUUGACAACAGUUCUAUAUCUCGAGAUAUAGAACUGUUGUCAA
GGCUUUUUUG

### SEQ ID No. 10:

Nucleotide sequence of mature antisense sequence derived from shRNA targeting nucleotide sequence encoding homo sapiens SLC38A9 (Sentor)

AUAUAGAACUGUUGUCAAGGC

### SEQ ID No. 11:

Nucleotide target sequence of human SLC38A9 mRNA (corresponding to nt 1931-1951 of human SLC38A9 mRNA (NM_173514.3))

GCCTTGACAACAGTTCTATAT

### SEQ ID No. 12:

Nucleotide sequence encoding homo sapiens SLC38A9 (Sentor) (Isoform 2: NM_001258286.1->NP_001245215.1, open reading frame): >SLC38A9|NM_001258286.1|CDS

### SEQ ID No. 13:

Nucleotide sequence encoding homo sapiens SLC38A9 (Sentor) (Isoform 2: NM_001258286.1->NP_001245215.1, mRNA): >SLC38A9|NM_001258286.1|mRNA

### SEQ ID No. 14:

Amino acid sequence of homo sapiens SLC38A9 isoform 2 (Isoform2: NP_001245215.1):

### SEQ ID No. 15:

Nucleotide sequence encoding homo sapiens SLC38A9 (Sentor) (Isoform3: NM_001258287.1

->NP_001245216.1, open reading frame):

### >SLC38A9|NM_001258287.1|CDS

### SEQ ID No. 16:

Nucleotide sequence encoding homo sapiens SLC38A9 (Sentor) (Isoform3: NM_001258287.1

->NP_001245216.1, mRNA):
>SLC38A9|NM_001258287.1|mRNA

### SEQ ID No. 17:

Amino acid sequence of homo sapiens SLC38A9 isoform 3 (NP_001245216.1):

### SEQ ID No. 18:

Nucleotide sequence encoding homo sapiens SLC38A9 (Sentor) (Isoform 4: NM_001282429.1->NP_001269358.1, open reading frame):

### >SLC38A9|NM_001282429.1|CDS

### SEQ ID No. 19:

Nucleotide sequence encoding homo sapiens SLC38A9 (Sentor) (Isoform 4: NM_001282429.1->NP_001269358.1, mRNA):

>SLC38A9|NM_001282429.1|mRNA

### SEQ ID No. 20:

Amino acid sequence of homo sapiens SLC38A9 isoform 4 (NP_001269358.1):

### SEQ ID No. 21:

Nucleotide sequence encoding the N-terminal cytoplasmic region of homo sapiens SLC38A9 (Sentor) isoform 1:

### SEQ ID No. 22:

Amino acid sequence of the N-terminal cytoplasmic region of homo sapiens SLC38A9 (Sentor) isoform 1.

### SEQ ID No. 23:

Nucleotide sequence encoding homo sapiens Deptor

>Deptor|NM_022783.2|mRNA

### SEQ ID No. 24:

Nucleotide sequence encoding homo sapiens Deptor

### >Deptor|NM_022783.2|CDS

### SEQ ID No. 25:

Amino acid sequence of homo sapiens Deptor

>Deptor|NM_022783.2|protein

### SEQ ID No. 26:

Nucleotide sequence encoding homo sapiens mLST8

>mLST8|NM_001199173.1|mRNA

### SEQ ID No. 27:

Nucleotide sequence encoding homo sapiens mLST8

>mLST8|NM_001199173.1|CDS

### SEQ ID No. 28:

Amino acid sequence of homo sapiens mLST8

>mLST8|NM_001199173.1|protein

### SEQ ID No. 29:

Nucleotide sequence encoding homo sapiens mLST8

>mLST8|NM_001199174.1|mRNA

### SEQ ID No. 30:

Nucleotide sequence encoding homo sapiens mLST8

>mLST8|NM_001199174.1|CDS

### SEQ ID No. 31:

Amino acid sequence of homo sapiens mLST8

>mLST8|NM_001199174.1|protein

### SEQ ID No. 32:

Nucleotide sequence encoding homo sapiens mLST8

>mLST8|NM_001199175.1|mRNA

### SEQ ID No. 33:

Nucleotide sequence encoding homo sapiens mLST8

>mLST8|NM_001199175.1|CDS

### SEQ ID No. 34:

Amino acid sequence of homo sapiens mLST8

>mLST8|NM_001199175.1|protein

### SEQ ID No. 35:

Nucleotide sequence encoding homo sapiens mLST8

>mLST8|NM_022372.4|mRNA

### SEQ ID No. 36:

Nucleotide sequence encoding homo sapiens mLST8

>mLST8|NM_022372.41 CDS

### SEQ ID No. 37:

Amino acid sequence of homo sapiens mLST8

>mLST8|NM_022372.4|protein

### SEQ ID No. 38:

Nucleotide sequence encoding homo sapiens mTOR

>mTOR|NM_004958.3|mRNA

### SEQ ID No. 39:

Nucleotide sequence encoding homo sapiens mTOR

>mTOR|NM_004958.3|CDS

### SEQ ID No. 40:

Amino acid sequence of homo sapiens mTOR

>mTOR|NM_004958.3|protein

### SEQ ID No. 41:

Nucleotide sequence encoding homo sapiens Raptor

>Raptor|NM_020761.2|mRNA

### SEQ ID No. 42:

Nucleotide sequence encoding homo sapiens Raptor

### >Raptor|NM_020761.2|CDS

### SEQ ID No. 43:

Amino acid sequence of homo sapiens Raptor

>Raptor|NM_020761.2|protein

### SEQ ID No. 44:

Nucleotide sequence encoding homo sapiens PRAS40

>PRAS40|NM_001098632.1|mRNA

### SEQ ID No. 45:

Nucleotide sequence encoding homo sapiens PRAS40

### >PRAS40|NM_001098632.1|CDS

### SEQ ID No. 46:

Amino acid sequence of homo sapiens PRAS40

>PRAS40|NM_001098632.1|protein

### SEQ ID No. 47:

Nucleotide sequence encoding homo sapiens PRAS40

>PRAS40|NM_001098633.2|mRNA

### SEQ ID No. 48:

Nucleotide sequence encoding homo sapiens PRAS40

### >PRAS40|NM_001098633.2|CDS

### SEQ ID No. 49:

Amino acid sequence of homo sapiens PRAS40

>PRAS40|NM_001098633.2|protein

### SEQ ID No. 50:

Nucleotide sequence encoding homo sapiens PRAS40

>PRAS40|NM_032375.4|mRNA

### SEQ ID No. 51:

Nucleotide sequence encoding homo sapiens PRAS40

### >PRAS40|NM_032375.4|CDS

### SEQ ID No. 52:

Amino acid sequence of homo sapiens PRAS40

>PRAS40|NM_032375.4|protein

### LIST OF REFERENCES

1 Dibble, C. C. & Manning, B. D. Signal integration by mTORC1 coordinates nutrient input with biosynthetic output. Nature cell biology 15, 555-564, doi:10.1038/ncb2763 (2013).
2 Alessi, D. R. & Kulathu, Y. Structural biology: Security measures of a master regulator. Nature 497, 193-194, doi:10.1038/nature12101 (2013).
3 Kim, E., Goraksha-Hicks, P., Li, L., Neufeld, T. P. & Guan, K. L. Regulation of TORC1 by RAG GTPases in nutrient response. Nature cell biology 10, 935-945, doi:10.1038/ncb1753 (2008).
4 Sancak, Y. et al. The RAG GTPases bind RAPTOR and mediate amino acid signaling to mTORC1. Science 320, 1496-1501, doi:10.1126/science.1157535 (2008).
5 Sancak, Y. et al. Ragulator-Rag complex targets mTORC1 to the lysosomal surface and is necessary for its activation by amino acids. Cell 141, 290-303, doi:10.1016/j.cell.2010.02.024 (2010).
6 Jewell, J. L., Russell, R. C. & Guan, K. L. Amino acid signalling upstream of mTOR. Nature reviews. Molecular cell biology 14, 133-139, doi:10.1038/nrm3522 (2013).
7 Cornu, M., Albert, V. & Hall, M. N. mTOR in aging, metabolism, and cancer. Current opinion in genetics & development 23, 53-62, doi:10.1016/j.gde.2012.12.005 (2013).
8 Laplante, M. & Sabatini, D. M. mTOR signaling in growth control and disease. Cell 149, 274-293, doi:10.1016/j.cell.2012.03.017 (2012).
9 Hara, K. et al Amino acid sufficiency and mTOR regulate p70 S6 kinase and eIF-4E BP1 through a common effector mechanism. The Journal of biological chemistry 273, 14484-14494 (1998).
10 Han, J. M. et al. Leucyl-tRNA synthetase is an intracellular leucine sensor for the mTORC1-signaling pathway. Cell 149, 410-424, doi:10.1016/j.cell.2012.02.044 (2012).
11 Bonfils, G. et al. Leucyl-tRNA synthetase controls TORC1 via the EGO complex. Molecular cell 46, 105-110, doi:10.1016/j.molce1.2012.02.009 (2012).
12 Duran, R. V. et al. Glutaminolysis activates Rag-mTORC1 signaling. Molecular cell 47, 349-358, doi:10.1016/j.molce1.2012.05.043 (2012).
13 Zoncu, R. et al. mTORC1 senses lysosomal amino acids through an inside-out mechanism that requires the vacuolar H(+)-ATPase. Science 334, 678-683, doi:10.1126/science.1207056 (2011).
14 Bar-Peled, L., Schweitzer, L. D., Zoncu, R. & Sabatini, D. M. Ragulator is a GEF for the RAG GTPases that signal amino acid levels to mTORC1. Cell 150, 1196-1208, doi:10.1016/j.cell.2012.07.032 (2012).
15 Nicklin, P. et al. Bidirectional transport of amino acids regulates mTOR and autophagy. Cell 136, 521-534, doi:10.1016/j.cell.2008.11.044 (2009).
16 Hediger, M. A., Clemencon, B., Burrier, R. E. & Bruford, E. A. The ABCs of membrane transporters in health and disease (SLC series): introduction. Molecular aspects of medicine 34, 95-107, doi:10.1016/j.mam.2012.12.009 (2013).
17 Uhlen, M. et al. Towards a knowledge-based Human Protein Atlas. Nature biotechnology 28, 1248-1250, doi:10.1038/nbt1210-1248 (2010).
18 Chapel, A. et al. An extended proteome map of the lysosomal membrane reveals novel potential transporters. Molecular & cellular proteomics: MCP 12, 1572-1588, doi:10.1074/mcp.M112.021980 (2013).
19 Schioth, H. B., Roshanbin, S., Hagglund, M. G. & Fredriksson, R. Evolutionary origin of amino acid transporter families SLC32, SLC36 and SLC38 and physiological, pathological and therapeutic aspects. Molecular aspects of medicine 34, 571-585, doi:10.1016/j.mam 2012.07.012 (2013).
20 Mackenzie, B. & Erickson, J. D. Sodium-coupled neutral amino acid (System N/A) transporters of the SLC38 gene family. Pflugers Archiv: European journal of physiology 447, 784-795, doi:10.1007/s00424-003-1117-9 (2004).
21 Shaw, R. J. & Cantley, L. C. Ras, PI(3)K and mTOR signalling controls tumour cell growth. Nature 441, 424-430, doi:10.1038/nature04869 (2006).
22 Kocher, T. & Superti-Furga, G. Mass spectrometry-based functional proteomics: from molecular machines to protein networks. Nature methods 4, 807-815, doi:10.1038/nmeth1093 (2007).
23 Tsun, Z. Y. et al. The Folliculin Tumor Suppressor Is a GAP for the RAGC/D GTPases That Signal Amino Acid Levels to mTORC1. Molecular cell, doi:10.1016/j.molce1.2013.09.016 (2013).
24 Hagglund, M. G. et al. Identification of SLC38A7 (SNAT7) protein as a glutamine transporter expressed in neurons. The Journal of biological chemistry 286, 20500-20511, doi:10.1074/jbc.M110.162404 (2011).
25 Indiveri, C., Galluccio, M., Scalise, M. & Pochini, L. Strategies of bacterial over expression of membrane transporters relevant in human health: the successful case of the three members of OCTN subfamily. Molecular biotechnology 54, 724-736, doi:10.1007/s12033-012-9586-8 (2013).
26 Gaber, R. F., Ottow, K., Andersen, H. A. & Kielland-Brandt, M. C. Constitutive and hyperresponsive signaling by mutant forms of Saccharomyces cerevisiae amino acid sensor Ssy1. Eukaryotic cell 2, 922-929 (2003).
27 Goberdhan, D. C., Meredith, D., Boyd, C. A. & Wilson, C. PAT-related amino acid transporters regulate growth via a novel mechanism that does not require bulk transport of amino acids. Development 132, 2365-2375, doi:10.1242/dev.01821 (2005).
28 Dann, S. G., Selvaraj, A. & Thomas, G. mTOR Complex1-S6K1 signaling: at the crossroads of obesity, diabetes and cancer. Trends in molecular medicine 13, 252-259, doi:10.1016/j.molmed.2007.04.002 (2007).
29 Beugnet, A., Tee, A. R., Taylor, P. M. & Proud, C. G. Regulation of targets of mTOR (mammalian target of rapamycin) signalling by intracellular amino acid availability. The Biochemical journal 372, 555-566, doi:10.1042/BJ20021266 (2003).
30 Price, D. J., Nemenoff, R. A. & Avruch, J. Purification of a hepatic S6 kinase from cycloheximide-treated Rats. The Journal of biological chemistry 264, 13825-13833 (1989).
31 Gaccioli, F. et al Amino acid starvation induces the SNAT2 neutral amino acid transporter by a mechanism that involves eukaryotic initiation factor 2alpha phosphorylation and cap-independent translation. The Journal of biological chemistry 281, 17929-17940, doi:10.1074/jbc.M600341200 (2006).
32 Giacomini, K. M. et al. Membrane transporters in drug development. Nature reviews. Drug discovery 9, 215-236, doi:10.1038/nrd3028 (2010).
33 Snijder, B. et al. Population context determines cell-to-cell variability in endocytosis and virus infection. Nature 461, 520-523, doi:10.1038/nature08282 (2009).
34 Mellacheruvu, D. et al. The CRAPome: a contaminant repository for affinity purification-mass spectrometry data. Nature methods 10, 730-736, doi:10.1038/nmeth.2557 (2013).
35 Choi, H. et al. SAINT: probabilistic scoring of affinity purification-mass spectrometry data. Nature methods 8, 70-73, doi:10.1038/nmeth.1541 (2011).

### LIST OF REFERENCES CITED IN THE MATERIAL & METHODS SECTION OF THE EXAMPLE

1 Glatter, T., Wepf, A., Aebersold, R. & Gstaiger, M. An integrated workflow for charting the human interaction proteome: insights into the PP2A system. Molecular systems biology 5, 237, doi:10.1038/msb.2008.75 (2009).
2 Pichlmair, A. et al. Viral immune modulators perturb the human molecular network by common and unique strategies. Nature 487, 486-490, doi:10.1038/nature11289 (2012).
3 Varjosalo, M. et al. Interlaboratory reproducibility of large-scale human protein-complex analysis by standardized AP-MS. Nature methods 10, 307-314, doi:10.1038/nmeth.2400 (2013).
4 Colinge, J., Masselot, A., Giron, M., Dessingy, T. & Magnin, J. OLAV: towards high-throughput tandem mass spectrometry data identification. Proteomics 3, 1454-1463, doi:10.1002/pmic.200300485 (2003).
5 Bennett, K. L. et al. Proteomic analysis of human cataract aqueous humour: Comparison of one-dimensional gel LCMS with two-dimensional LCMS of unlabelled and iTRAQ(R)-labelled specimens. Journal of proteomics 74, 151-166, doi:10.1016/j.jprot.2010.10.002 (2011).
6 Snijder, B. et al. Population context determines cell-to-cell variability in endocytosis and virus infection. Nature 461, 520-523, doi:10.1038/nature08282 (2009).
7 Galluccio, M. et al. Over-expression in E. coli and purification of the human OCTN1 transport protein. Protein expression and purification 68, 215-220, doi:10.1016/j.pep.2009.06.015 (2009).
8 Pochini, L., Scalise, M., Galluccio, M., Amelio, L. & Indiveri, C. Reconstitution in liposomes of the functionally active human OCTN1 (SLC22A4) transporter overexpressed in Escherichia coli. The Biochemical journal 439, 227-233, doi:10.1042/BJ20110544 (2011).

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. An antagonist of SLC38A9 for use in treating a disease associated with mTORC1 activation.

2. A method for treating, preventing or ameliorating a disease associated with mTORC1 activation comprising the administration of an antagonist of SLC38A9 to a subject in need of such a treatment, prevention or amelioration.

3. The antagonist of claim 1, or the method of claim 2, wherein said disease associated with mTORC1 activation is a proliferative disease, a metabolic disorder, a disorder of the immune system, a disorder causing premature aging, an ophthalmic disorder or a neurological disorder.

4. The antagonist of claim 3, or the method of claim 3, wherein said proliferative disease is a cancerous disease or a benign proliferative disease.

5. The antagonist of claim 4, or the method of claim 4, wherein said cancerous disease is selected from the group consisting of lung cancer, breast cancer, bladder cancer, pancreatic cancer, ovarian cancer, colon carcinoma, leukemia, lymphoma, melanoma, esophageal cancer and stomach cancer.

6. The antagonist of claim 3, or the method of claim 3, wherein said metabolic disorder is overweight (pre-obesity), obesity or diabetes.

7. The antagonist of claim 6, or the method of claim 6, wherein said overweight (pre-obesity) is defined as a body mass index (BMI) between 25 to 30 kg/m² of the subject to be treated.

8. The antagonist of claim 6, or the method of claim 6, wherein said obesity is defined as a body mass index (BMI) of higher than 30 kg/m² of the subject to be treated.

9. The antagonist of claim 6, or the method of claim 6, wherein said diabetes is type 2 diabetes.

10. The antagonist of any one of claims 6 to 9, or the method of any one of claims 6 to 9, wherein said disease is characterized as 20% or more body fat in the subject to be treated.

11. The antagonist of any one of claims 1 and 3 to 10, or the method of any one of claims 2 to 10, wherein said SLC38A9 is selected from the group consisting of
(a) a polypeptide comprising an amino acid encoded by a nucleic acid molecule having the nucleic acid sequence as depicted in SEQ ID NO: 1, 2 or 4;
(b) a polypeptide having an amino acid sequence as depicted in SEQ ID NO:3;
(c) a polypeptide encoded by a nucleic acid molecule encoding a peptide having an amino acid sequence as depicted in SEQ ID NO: 3;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

12. The antagonist of any one of claims 1 and 3 to 11, or the method of any one of claims 2 to 11, wherein said antagonist is selected from the group consisting of binding molecules, small molecule drugs, siRNA, shRNA, miRNA, dsRNA, stRNA and antisense molecules.

13. The antagonist of claim 12, or the method of claim 12, wherein said binding molecule is selected from the group consisting of aptamers and intramers.

14. The antagonist of claim 12 or 13, or the method of claim 12 or 13, wherein said binding molecule specifically binds to SLC38A9, particularly SLC38A9 as defined in claim 11.

15. The antagonist of claim 12 or 13, or the method of claim 12 or 13, wherein said binding molecule, siRNA, shRNA, miRNA, dsRNA, stRNA, or antisense molecule targets a nucleic acid molecule having a sequence encoding SLC38A9.

16. The antagonist of claim 15, or the method of claim 15, wherein said nucleic acid is selected from the group consisting of
(a) a nucleic acid encoding a polypeptide comprising an amino acid sequence as depicted in SEQ ID NO: 3;
(b) a nucleic acid comprising a nucleotide sequence as depicted in SEQ ID NO: 4;
(c) a nucleic acid hybridizing under stringent conditions to the complementary strand of the nucleic acid as defined in (a) or (b);
(d) a nucleic acid comprising a nucleotide sequence with at least 70% identity to the nucleotide sequence of the nucleic acids of any one of (a) to (c); and
(e) a nucleic acid comprising a nucleotide sequence which is degenerate as a result of the genetic code to the nucleotide sequence of a nucleic acid of any one of (a) to (d).

17. The antagonist of claim 16, or the method of claim 16, wherein said nucleic acid comprises a nucleotide as shown in SEQ ID NO. 63, SEQ ID NO. 64, SEQ ID NO. 65, or SEQ ID NO. 66.

18. The antagonist of any one of claims 12 and 15 to 17, or the method of any one of claims 12 and 15 to 17, wherein said siRNA comprises a nucleic acid molecule comprising at least eight contiguous bases having a sequence as shown in the sequence of SEQ ID NO: 5, 6, 7 or 8.

19. The antagonist of claim 18, or the method of claim 18, wherein up to 10% of the contiguous bases are non-complementary to the target sequence.

20. The antagonist of claim 18 or 19, or the method of claim 18 or 19, wherein said siRNA further comprises at least one base at the 5′ end and/or at least one base at the 3′ end.

21. The antagonist of any one of claims 12 and 15 to 20, or the method of any one of claims 12 and 15 to 20, wherein said siRNA consists of a molecule as shown in SEQ ID NO: 5, 6, 7 or 8.

22. The antagonist of any one of claims 12 and 15 to 17, or the method of any one of claims 12 and 15 to 17, wherein said shRNA comprises a nucleic acid molecule comprising at least eight contiguous nucleotides having a sequence as shown in the sequence of SEQ ID NO: 9 or 10.

23. The antagonist of any one of claims 1 and 3 to 22, or the method of any one of claims 2 to 22, wherein the antagonist is a selective antagonist of SLC38A9.

24. An antagonist of SLC38A9.

25. An antagonist of SLC38A9 for use in medicine.

26. The antagonist of any claim 24 or 25, wherein said SLC38A9 is selected from the group consisting of
(a) a polypeptide comprising an amino acid encoded by a nucleic acid molecule having the nucleic acid sequence as depicted in SEQ ID NO: 1, 2 or 4;
(b) a polypeptide having an amino acid sequence as depicted in SEQ ID NO:3;
(c) a polypeptide encoded by a nucleic acid molecule encoding a peptide having an amino acid sequence as depicted in SEQ ID NO: 3;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

27. The antagonist of any one of claims 24 to 26, wherein said antagonist is selected from the group consisting of binding molecules, small molecule drugs, siRNA, shRNA, miRNA, dsRNA, stRNA and antisense molecules.

28. The antagonist of claim 27, wherein said binding molecule is selected from the group consisting of aptamers and intramers.

29. The antagonist of claim 27 or 28, wherein said binding molecule specifically binds to SLC38A9, particularly SLC38A9 as defined in claim 26.

30. The antagonist of claim 27 or 28, wherein said binding molecule, siRNA, shRNA, miRNA, dsRNA, stRNA, or antisense molecule targets a nucleic acid molecule having a sequence encoding SLC38A9.

31. The antagonist of claim 30, wherein said nucleic acid is selected from the group consisting of
(a) a nucleic acid encoding a polypeptide comprising an amino acid sequence as depicted in SEQ ID NO: 3;
(b) a nucleic acid comprising a nucleotide sequence as depicted in SEQ ID NO: 4;
(c) a nucleic acid hybridizing under stringent conditions to the complementary strand of the nucleic acid as defined in (a) or (b);
(d) a nucleic acid comprising a nucleotide sequence with at least 70% identity to the nucleotide sequence of the nucleic acids of any one of (a) to (c); and
(e) a nucleic acid comprising a nucleotide sequence which is degenerate as a result of the genetic code to the nucleotide sequence of a nucleic acid of any one of (a) to (d).

32. The antagonist of claim 31, wherein said nucleic acid comprises a nucleotide as shown in SEQ ID NO. 63, SEQ ID NO. 64, SEQ ID NO. 65, or SEQ ID NO. 66.

33. The antagonist of any one of claims 27 and 30 to 32, wherein said siRNA comprises a nucleic acid molecule comprising at least eight contiguous bases having a sequence as shown in the sequence of SEQ ID NO: 5, 6, 7 or 8.

34. The antagonist of claim 33, wherein up to 10% of the contiguous bases are non-complementary to the target sequence.

35. The antagonist of claim 33 or 34, wherein said siRNA further comprises at least one base at the 5′ end and/or at least one base at the 3′ end.

36. The antagonist of any one of claims 27 and 30 to 35, wherein said siRNA consists of a molecule as shown in SEQ ID NO: 5, 6, 7 or 8.

37. The antagonist of any one of claims 27 and 30 to 32, wherein said shRNA comprises a nucleic acid molecule comprising at least eight contiguous nucleotides having a sequence as shown in the sequence of SEQ ID NO: 9 or 10.

38. The antagonist of any one of claims 24 to 37, wherein the antagonist is a selective antagonist of SLC38A9.

39. Method for assessing the activity of a candidate molecule suspected of being an antagonist of SLC38A9 comprising the steps of:
(a) contacting a cell, tissue or a non-human animal comprising SLC38A9 with said candidate molecule;
(b) detecting a decrease in activity of said SLC38A9; and
(c) selecting a candidate molecule that decreases activity of said SLC38A9;
wherein a decrease of the SLC38A9 activity is indicative for the capacity of the selected molecule to antagonise mTORC1.

40. The method of claim 39, wherein said SLC38A9 is selected from the group consisting of
(a) a polypeptide comprising an amino acid encoded by a nucleic acid molecule having the nucleic acid sequence as depicted in SEQ ID NO: 1, 2 or 4;
(b) a polypeptide having an amino acid sequence as depicted in SEQ ID NO:3;
(c) a polypeptide encoded by a nucleic acid molecule encoding a peptide having an amino acid sequence as depicted in SEQ ID NO: 3;
(d) a polypeptide comprising an amino acid encoded by a nucleic acid molecule hybridizing under stringent conditions to the complementary strand of nucleic acid molecules as defined in (a) or (c);
(e) a polypeptide having at least 70% identity to the polypeptide of any one of (a) to (d); and
(f) a polypeptide comprising an amino acid encoded by a nucleic acid molecule being degenerate as a result of the genetic code to the nucleotide sequence of a nucleic acid molecule as defined in (a), (c) and (d).

41. The method of claim 39 or 40, wherein said candidate molecule is selected from the group consisting of binding molecules, small molecule drugs, siRNA, shRNA, miRNA, dsRNA, stRNA and antisense molecules.

42. The method of claim 41, wherein said binding molecule is selected from the group consisting of aptamers and intramers.

43. The method of claim 41 or 42, wherein said binding molecule specifically binds to SLC38A9, particularly SLC38A9 as defined in claim 40.

44. The method of claim 41 or 42, wherein said binding molecule, siRNA, shRNA, miRNA, dsRNA, stRNA, or antisense molecule targets a nucleic acid molecule having a sequence encoding SLC38A9.

45. The method of claim 44, wherein said nucleic acid is selected from the group consisting of
(a) a nucleic acid encoding a polypeptide comprising an amino acid sequence as depicted in SEQ ID NO: 3;
(b) a nucleic acid comprising a nucleotide sequence as depicted in SEQ ID NO: 4;
(c) a nucleic acid hybridizing under stringent conditions to the complementary strand of the nucleic acid as defined in (a) or (b);
(d) a nucleic acid comprising a nucleotide sequence with at least 70% identity to the nucleotide sequence of the nucleic acids of any one of (a) to (c); and
(e) a nucleic acid comprising a nucleotide sequence which is degenerate as a result of the genetic code to the nucleotide sequence of a nucleic acid of any one of (a) to (d).

46. The method of claim 45, wherein said nucleic acid comprises a nucleotide as shown in SEQ ID NO. 63, SEQ ID NO. 64, SEQ ID NO. 65, or SEQ ID NO. 66.

47. The method of any one of claims 41 and 44 to 46, wherein said siRNA comprises a nucleic acid molecule comprising at least eight contiguous bases having a sequence as shown in the sequence of SEQ ID NO: 5, 6, 7 or 8.

48. The method of claim 47, wherein up to 10% of the contiguous bases are non-complementary to the target sequence.

49. The method of claim 47 or 48, wherein said siRNA further comprises at least one base at the 5′ end and/or at least one base at the 3′ end.

50. The method of any one of claims 41 and 44 to 46, wherein said siRNA consists of a molecule as shown in SEQ ID NO: 5, 6, 7 or 8.

51. The method of any one of claims 41 and 44 to 46, wherein said shRNA comprises a nucleic acid molecule comprising at least eight contiguous nucleotides having a sequence as shown in the sequence of SEQ ID NO: 9 or 10.

52. The method of any one of claims 39 to 51, wherein the candidate molecule is suspected of being a selective antagonist of SLC38A9.


SEQUENCE LISTING (114 sequences)

SEQ ID NO: 1 - DNA, 2543 bp, Homo sapiens
  Nucleotide sequence encoding homo sapiens SLC 38A9 (Sentor) (NCBI gene ID 153129 Isoform 1 NM_173514.3 ->NP_775785.2)
aggctattgccgcttcctgtttggactaggagccccgcggtcgcaaattgcgagaatttgggaagggaatctttatcttctcgtctagttctccgaggttgaaggctcggcctgctcagagaaggaaactgaggtccaccgagttggagaaacctactcaacaccaggactaacttcttcagtgcttagagtgtgagaaaaatggcaaatatgaatagtgattctaggcatcttggcacctctgaggtagatcatgaaagagatcctggacctatgaatatccagtttgagccatcggatctaagatccaaaaggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaaggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctaggcacagtgtctgtcctttatttgattttccttgtcacctttaaggctgttcgcttgggatttcatttggaatttcattggtttataccaacagaattttttgtaccagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtgaaatacctcaactgtttttttcaagagctctcatgatattttgagccttgacaacagttctatataaattcacttgtaaatgctgctgttgtgtaattctaaatattttctaagataatttgaaagcaagggaaatagtggccccttaatgagtatttttttattggggtggggaaaggggcaaaaagaatgatcttagtgtctttacctttctcatattaactcacctctttattctgtggtcttttctgaatagaaatgtatgccctaggaagaaatcatgctgggttttgcttttagagataaaaggtggtggatttattttgcctgcagtaaagattctcagggtgtcagagcagcatattgtcaaatcctgcttctgttttatgtttcagtgtattcactttcattttcttacttactagaccatttctgcagtttgcccaaacctctactgtttgggacagtaagccaaatacctcatttttaaaaagaagttttcatggcatcagtgttaataaagtacatttttaactgagtcttaatctctatttgaagaaaaagtagagacaaaagtaatgtcaatgtaatccccaggatcatgaaatgtatacaaaataaataaagtaggagagtttgttgctgtctaaaaaaaaa

SEQ ID NO: 2 - DNA, 1686 bp, Homo sapiens
  Nucleotide sequence encoding homo sapiens SLC38A9 (Sentor) (NCBI geneID 153129; Isoform1  NM_173514.3 -> NP_775785.2, open reading frame)
atggcaaatatgaatagtgattctaggcatcttggcacctctgaggtagatcatgaaagagatcctggacctatgaatatccagtttgagccatcggatctaagatccaaaaggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaaggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctaggcacagtgtctgtcctttatttgattttccttgtcacctttaaggctgttcgcttgggatttcatttggaatttcattggtttataccaacagaattttttgtaccagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtga

SEQ ID NO: 3 - protein, 561 aa, Homo sapiens
  Amino acid sequence of homo sapiens SLC38A9 isoform1 (NP_775785.2)
MANMNSDSRHLGTSEVDHERDPGPMNIQFEPSDLRSKRPFCIEPTNIVNVNHVIQRVSDHASAMNKRIHYYSRLTTPADKALIAPDHVVPAPEECYVYSPLGSAYKLQSYTEGYGKNTSLVTIFMIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILGTVSVLYLIFLVTFKAVRLGFHLEFHWFIPTEFFVPEIRFQFPQLTGVLTLAFFIHNCIITLLKNNKKQENNVRDLCIAYMLVTLTYLYIGVLVFASFPSPPLSKDCIEQNFLDNFPSSDTLSFIARIFLLFQMMTVYPLLGYLARVQLLGHIFGDIYPSIFHVLILNLIIVGAGVIMACFYPNIGGIIRYSGAACGLAFVFIYPSLIYIISLHQEERLTWPKLIFHVFIIILGVANLIVQFFM

SEQ ID NO: 4 - RNA, 2543 nt, Homo sapiens
  Nucleotide sequence encoding homo sapiens SLC38A9 (Sentor) (NCBI geneID 153129; Isoform1  NM_173514.3 -> NP_775785.2, open reading frame, mRNA)
aggcuauugccgcuuccuguuuggacuaggagccccgcggucgcaaauugcgagaauuugggaagggaaucuuuaucuucucgucuaguucuccgagguugaaggcucggccugcucagagaaggaaacugagguccaccgaguuggagaaaccuacucaacaccaggacuaacuucuucagugcuuagagugugagaaaaauggcaaauaugaauagugauucuaggcaucuuggcaccucugagguagaucaugaaagagauccuggaccuaugaauauccaguuugagccaucggaucuaagauccaaaaggccuuucuguauagagcccacaaacaucgugaaugugaaucaugucauucagaggguuagugaccaugccucugccaugaacaagagaauucauuacuacagccggcucaccacuccugcagacaaggcacugauugccccagaccauguaguuccagcuccagaagagugcuauguguauaguccauugggcucugcuuauaaacuucaaaguuacacugaaggauacgguaaaaacaccaguuuaguaaccauuuuuaugauuuggaauaccaugaugggaacaucuauacuaagcauuccuuggggcauaaaacaggcuggauuuacuacuggaaugugugucaucauacugaugggccuuuuaacacuuuauugcugcuacagaguagugaaaucacggacuaugauguuuucguuggauaccacuagcugggaauauccagaugucugcagacauuauuucggcuccuuugggcaguggucgagucuccuuuucuccuuggugucucucauuggagcaaugauaguuuauugggugcuuaugucaaauuuucuuuuuaauacuggaaaguuuauuuuuaauuuuauucaucacauuaaugacacagacacuauacugaguaccaauaauagcaacccugugauuuguccaagugccgggaguggaggccauccugacaacagcucuaugauuuucuaugccaaugacacaggagcccaacaguuugaaaaguggugggauaaguccaggacaguccccuuuuaucuuguagggcuccuccucccacugcucaauuucaagucuccuucauuuuuuucaaaauuuaauauccuaggcacagugucuguccuuuauuugauuuuccuugucaccuuuaaggcuguucgcuugggauuucauuuggaauuucauugguuuauaccaacagaauuuuuuguaccagagauaagauuucaguuuccacagcugacuggagugcuuacccuugcuuuuuuuauucauaauuguaucaucacacucuugaagaacaacaagaaacaagaaaacaaugugagggacuugugcauugcuuauaugcuggugacauuaacuuaucucuauauuggaguccugguuuuugcuucauuuccuucaccaccauuauccaaagauuguauugagcagaauuuuuuagacaacuucccuagcagugacacccuguccuucauugcaaggauauuccugcuguuccagaugaugacuguauacccacucuuaggcuaccuggcucguguccagcuuuugggccauaucuucggugacauuuauccuagcauuuuccaugugcugauucuuaaucuaauuauugugggagcuggagugaucauggccuguuucuacccaaacauaggagggaucauaagauauucaggagcagcauguggacuggccuuuguauucauauacccaucucucaucuauauaauuucccuccaccaagaagagcgucugacauggccuaaauuaaucuuccacguuuucaucaucauuuugggcguggcuaaccugauuguucaguuuuuuaugugaaauaccucaacuguuuuuuucaagagcucucaugauauuuugagccuugacaacaguucuauauaaauucacuuguaaaugcugcuguuguguaauucuaaauauuuucuaagauaauuugaaagcaagggaaauaguggccccuuaaugaguauuuuuuuauugggguggggaaaggggcaaaaagaaugaucuuagugucuuuaccuuucucauauuaacucaccucuuuauucuguggucuuuucugaauagaaauguaugcccuaggaagaaaucaugcuggguuuugcuuuuagagauaaaaggugguggauuuauuuugccugcaguaaagauucucagggugucagagcagcauauugucaaauccugcuucuguuuuauguuucaguguauucacuuucauuuucuuacuuacuagaccauuucugcaguuugcccaaaccucuacuguuugggacaguaagccaaauaccucauuuuuaaaaagaaguuuucauggcaucaguguuaauaaaguacauuuuuaacugagucuuaaucucuauuugaagaaaaaguagagacaaaaguaaugucaauguaauccccaggaucaugaaauguauacaaaauaaauaaaguaggagaguuuguugcugucuaaaaaaaaa

SEQ ID NO: 5 - RNA, 19 nt, Homo sapiens
  Nucleotide sequence of siRNA 1 (antisense sequence) targeting nucleotide sequence encoding homo sapiens SLC38A9 (Sentor)
uuaccguauccuucagugu

SEQ ID NO: 6 - RNA, 19 nt, Homo sapiens
  Nucleotide sequence of siRNA 2 (antisense sequence) targeting nucleotide sequence encoding homo sapiens SLC38A9 (Sentor)
uauucauagguccaggauc

SEQ ID NO: 7 - RNA, 19 nt, Homo sapiens
  Nucleotide sequence of siRNA 3 (antisense sequence) targeting nucleotide sequence encoding homo sapiens SLC38A9 (Sentor)
uauacacauagcacucuuc

SEQ ID NO: 8 - RNA, 19 nt, Homo sapiens
  Nucleotide sequence of siRNA 4 (antisense sequence) targeting nucleotide sequence encoding homo sapiens SLC38A9 (Sentor)
uaacccucugaaugacaug

SEQ ID NO: 9 - RNA, 59 nt, Homo sapiens
  Nucleotide sequence of shRNA targeting nucleotide sequence encoding homo sapiens SLC38A9 (Sentor)
ccgggccuugacaacaguucuauaucucgagauauagaacuguugucaaggcuuuuuug

SEQ ID NO: 10 - RNA, 21 nt, Homo sapiens
  Nucleotide sequence of mature antisense sequence derived from shRNA targeting nucleotide sequence encoding homo sapiens SLC38A9 (Sentor)
auauagaacuguugucaaggc

SEQ ID NO: 11 - RNA, 21 nt, Homo sapiens
  Nucleotide target sequence of human SLC38A9 mRNA (corresponding to nt 1931-1951 of human SLC38A9 mRNA (NM_173514.3))
gccttgacaacagttctatat

SEQ ID NO: 12 - DNA, 1497 bp, Homo sapiens
  Nucleotide sequence encoding homo sapiens SLC38A9 (Sentor) (Isoform 2  NM_001258286.1 -> NP_001245215.1, open reading frame)
atgaacaagagaattcattactacagccggctcaccactcctgcagacaaggcactgattgccccagaccatgtagttccagctccagaagagtgctatgtgtatagtccattgggctctgcttataaacttcaaagttacactgaaggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctaggcacagtgtctgtcctttatttgattttccttgtcacctttaaggctgttcgcttgggatttcatttggaatttcattggtttataccaacagaattttttgtaccagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtga

SEQ ID NO: 13 - RNA, 2693 nt, Homo sapiens
  Nucleotide sequence encoding homo sapiens SLC38A9 (Sentor) (Isoform 2  NM_001258286.1 -> NP_001245215.1, mRNA)
aagagaugaaagaugguugguuuaagcuaaucuugauaauccuauuuucauucgcacauacuuaaguuuuuagucuuucuuaaaacuagagguggaaaugugaaauuuaucacaugagaaauuuaucauugucauuuauaguggccaucagaaaagauaaugaaauaagacuuuugauucuuacuuauauaauccaaugucaguaaauauaagcacuaaugaaugcugacuugaaugcucaggguuuucuugaaaauucuaaauucugugauaaauaauugaccacugugacaacagaauguaagucauuaaagugcauuccauuacaguaaguuggcaggaguucaaacaguucucuguaaauuuguuuaacugcguagauuaaauggcaauauauuuucuauaaugaaacuuguugaaaugagagggaaucagcuauuuacaaucauggcauaacuaccuaggccuuucuguauagagcccacaaacaucgugaaugugaaucaugucauucagaggguuagugaccaugccucugccaugaacaagagaauucauuacuacagccggcucaccacuccugcagacaaggcacugauugccccagaccauguaguuccagcuccagaagagugcuauguguauaguccauugggcucugcuuauaaacuucaaaguuacacugaaggauacgguaaaaacaccaguuuaguaaccauuuuuaugauuuggaauaccaugaugggaacaucuauacuaagcauuccuuggggcauaaaacaggcuggauuuacuacuggaaugugugucaucauacugaugggccuuuuaacacuuuauugcugcuacagaguagugaaaucacggacuaugauguuuucguuggauaccacuagcugggaauauccagaugucugcagacauuauuucggcuccuuugggcaguggucgagucuccuuuucuccuuggugucucucauuggagcaaugauaguuuauugggugcuuaugucaaauuuucuuuuuaauacuggaaaguuuauuuuuaauuuuauucaucacauuaaugacacagacacuauacugaguaccaauaauagcaacccugugauuuguccaagugccgggaguggaggccauccugacaacagcucuaugauuuucuaugccaaugacacaggagcccaacaguuugaaaaguggugggauaaguccaggacaguccccuuuuaucuuguagggcuccuccucccacugcucaauuucaagucuccuucauuuuuuucaaaauuuaauauccuaggcacagugucuguccuuuauuugauuuuccuugucaccuuuaaggcuguucgcuugggauuucauuuggaauuucauugguuuauaccaacagaauuuuuuguaccagagauaagauuucaguuuccacagcugacuggagugcuuacccuugcuuuuuuuauucauaauuguaucaucacacucuugaagaacaacaagaaacaagaaaacaaugugagggacuugugcauugcuuauaugcuggugacauuaacuuaucucuauauuggaguccugguuuuugcuucauuuccuucaccaccauuauccaaagauuguauugagcagaauuuuuuagacaacuucccuagcagugacacccuguccuucauugcaaggauauuccugcuguuccagaugaugacuguauacccacucuuaggcuaccuggcucguguccagcuuuugggccauaucuucggugacauuuauccuagcauuuuccaugugcugauucuuaaucuaauuauugugggagcuggagugaucauggccuguuucuacccaaacauaggagggaucauaagauauucaggagcagcauguggacuggccuuuguauucauauacccaucucucaucuauauaauuucccuccaccaagaagagcgucugacauggccuaaauuaaucuuccacguuuucaucaucauuuugggcguggcuaaccugauuguucaguuuuuuaugugaaauaccucaacuguuuuuuucaagagcucucaugauauuuugagccuugacaacaguucuauauaaauucacuuguaaaugcugcuguuguguaauucuaaauauuuucuaagauaauuugaaagcaagggaaauaguggccccuuaaugaguauuuuuuuauugggguggggaaaggggcaaaaagaaugaucuuagugucuuuaccuuucucauauuaacucaccucuuuauucuguggucuuuucugaauagaaauguaugcccuaggaagaaaucaugcuggguuuugcuuuuagagauaaaaggugguggauuuauuuugccugcaguaaagauucucagggugucagagcagcauauugucaaauccugcuucuguuuuauguuucaguguauucacuuucauuuucuuacuuacuagaccauuucugcaguuugcccaaaccucuacuguuugggacaguaagccaaauaccucauuuuuaaaaagaaguuuucauggcaucaguguuaauaaaguacauuuuuaacugagucuuaaucucuauuugaagaaaaaguagagacaaaaguaaugucaauguaauccccaggaucaugaaauguauacaaaauaaauaaaguaggagaguuuguugcugucuaaaaaaaaa

SEQ ID NO: 14 - protein, 462 aa, Homo sapiens
  Amino acid sequence of homo sapiens SLC38A9 isoform 2 (Isoform2  NP_001245215.1)
MANMNSDSRHLGTSEVDHERDPGPMNIQFEPSDLRSKRPFCIEPTNIVNVNHVIQRVSDHASAMNKRIHYYSRLTTPADKALIAPDHVVPAPEECYVYSPLGSAYKLQSYTEGYGKNTSLVTIFMIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILGTVSVLYLIFLVTFKAVRLGFHLEFHWFIPTEFFVPEIRFQFPQLTGVLTLAFFIHNCIITLLKNNKKQENNVRDLCIAYMLVTLTYLYIGVLVFASFPSPPLSKDCIEQVRHRVPSLCDCVHFHVFIVGRVIQWQDITSDRPGF

SEQ ID NO: 15 - DNA, 1497 bp, Homo sapiens
  Nucleotide sequence encoding homo sapiens SLC38A9 (Sentor) (Isoform3  NM_001258287.1 -> NP_001245216.1, open reading frame)
atggctatttgcattttaacatggagaatccggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaaggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtga

SEQ ID NO: 16 - RNA, 2689 nt, Homo sapiens
  Nucleotide sequence encoding homo sapiens SLC38A9 (Sentor) (Isoform3  NM_001258287.1 -> NP_001245216.1, mRNA)
aagagaugaaagaugguugguuuaagcuaaucuugauaauccuauuuucauucgcacauacuuaaguuuuuagucuuucuuaaaacuagagguggaaaugugaaauuuaucacaugagaaauuuaucauugucauuuauaguggccaucagaaaagauaaugaaauaagacuuuugauucuuacuuauauaauccaaugucaguaaauauaagcacuaaugaaugcugacuugaaugcucaggguuuucuugaaaauucuaaauucugugauaaauaauugaccacugugacaacagaauguaagucauuaaagugcauuccauuacaguaaguuggcaggaguucaaacaguucucuguaaauuuguuuaacugcguagauuaaauggcaauauauuuucuauaaugaaacuuguugaaaugagagggaaucagcuauuuacaaucauggcauaacuaccuagggcucuguugucagcuugguauuauaauggaaaacaauuggauaugucagagacuuagucuauuacauuuccauggcuauuugcauuuuaacauggagaauccggccuuucuguauagagcccacaaacaucgugaaugugaaucaugucauucagaggguuagugaccaugccucugccaugaacaagagaauucauuacuacagccggcucaccacuccugcagacaaggcacugauugccccagaccauguaguuccagcuccagaagagugcuauguguauaguccauugggcucugcuuauaaacuucaaaguuacacugaaggauacgguaaaaacaccaguuuaguaaccauuuuuaugauuuggaauaccaugaugggaacaucuauacuaagcauuccuuggggcauaaaacaggcuggauuuacuacuggaaugugugucaucauacugaugggccuuuuaacacuuuauugcugcuacagaguagugaaaucacggacuaugauguuuucguuggauaccacuagcugggaauauccagaugucugcagacauuauuucggcuccuuugggcaguggucgagucuccuuuucuccuuggugucucucauuggagcaaugauaguuuauugggugcuuaugucaaauuuucuuuuuaauacuggaaaguuuauuuuuaauuuuauucaucacauuaaugacacagacacuauacugaguaccaauaauagcaacccugugauuuguccaagugccgggaguggaggccauccugacaacagcucuaugauuuucuaugccaaugacacaggagcccaacaguuugaaaaguggugggauaaguccaggacaguccccuuuuaucuuguagggcuccuccucccacugcucaauuucaagucuccuucauuuuuuucaaaauuuaauauccuagagauaagauuucaguuuccacagcugacuggagugcuuacccuugcuuuuuuuauucauaauuguaucaucacacucuugaagaacaacaagaaacaagaaaacaaugugagggacuugugcauugcuuauaugcuggugacauuaacuuaucucuauauuggaguccugguuuuugcuucauuuccuucaccaccauuauccaaagauuguauugagcagaauuuuuuagacaacuucccuagcagugacacccuguccuucauugcaaggauauuccugcuguuccagaugaugacuguauacccacucuuaggcuaccuggcucguguccagcuuuugggccauaucuucggugacauuuauccuagcauuuuccaugugcugauucuuaaucuaauuauugugggagcuggagugaucauggccuguuucuacccaaacauaggagggaucauaagauauucaggagcagcauguggacuggccuuuguauucauauacccaucucucaucuauauaauuucccuccaccaagaagagcgucugacauggccuaaauuaaucuuccacguuuucaucaucauuuugggcguggcuaaccugauuguucaguuuuuuaugugaaauaccucaacuguuuuuuucaagagcucucaugauauuuugagccuugacaacaguucuauauaaauucacuuguaaaugcugcuguuguguaauucuaaauauuuucuaagauaauuugaaagcaagggaaauaguggccccuuaaugaguauuuuuuuauugggguggggaaaggggcaaaaagaaugaucuuagugucuuuaccuuucucauauuaacucaccucuuuauucuguggucuuuucugaauagaaauguaugcccuaggaagaaaucaugcuggguuuugcuuuuagagauaaaaggugguggauuuauuuugccugcaguaaagauucucagggugucagagcagcauauugucaaauccugcuucuguuuuauguuucaguguauucacuuucauuuucuuacuuacuagaccauuucugcaguuugcccaaaccucuacuguuugggacaguaagccaaauaccucauuuuuaaaaagaaguuuucauggcaucaguguuaauaaaguacauuuuuaacugagucuuaaucucuauuugaagaaaaaguagagacaaaaguaaugucaauguaauccccaggaucaugaaauguauacaaaauaaauaaaguaggagaguuuguugcugucuaaaaaaaaa

SEQ ID NO: 17 - protein, 498 aa, Homo sapiens
  Amino acid sequence of homo sapiens SLC38A9 isoform 3 (NP_001245216.1 )
MAICILTWRIRPFCIEPTNIVNVNHVIQRVSDHASAMNKRIHYYSRLTTPADKALIAPDHVVPAPEECYVYSPLGSAYKLQSYTEGYGKNTSLVTIFMIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILEIRFQFPQLTGVLTLAFFIHNCIITLLKNNKKQENNVRDLCIAYMLVTLTYLYIGVLVFASFPSPPLSKDCIEQNFLDNFPSSDTLSFIARIFLLFQMMTVYPLLGYLARVQLLGHIFGDIYPSIFHVLILNLIIVGAGVIMACFYPNIGGIIRYSGAACGLAFVFIYPSLIYIISLHQEERLTWPKLIFHVFIIILGVANLIVQFFM

SEQ ID NO: 18 - DNA, 1314 bp, Homo sapiens
  Nucleotide sequence encoding homo sapiens SLC38A9 (Sentor) (Isoform 4  NM_001282429.1 -> NP_001269358.1, open reading frame)
atgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctaggcacagtgtctgtcctttatttgattttccttgtcacctttaaggctgttcgcttgggatttcatttggaatttcattggtttataccaacagaattttttgtaccagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtga

SEQ ID NO: 19 - RNA, 2410 nt, Homo sapiens
  Nucleotide sequence encoding homo sapiens SLC38A9 (Sentor) (Isoform 4  NM_001282429.1 -> NP_001269358.1, mRNA)
aggcuauugccgcuuccuguuuggacuaggagccccgcggucgcaaauugcgagaauuugggaagggaaucuuuaucuucucgucuaguucuccgagguugaaggcucggccugcucagagaaggaaacugagguccaccgaguuggagaaaccuacucaacaccaggacuaacuucuucagugcuuagagugugagaaaaauggcaaauaugaauagugauucuaggcaucuuggcaccucugagguagaucaugaaagagauccuggaccuaugaauauccaguuugagccaucggaucuaagauccaaaagauugccccagaccauguaguuccagcuccagaagagugcuauguguauaguccauugggcucugcuuauaaacuucaaaguuacacugaaggauacgguaaaaacaccaguuuaguaaccauuuuuaugauuuggaauaccaugaugggaacaucuauacuaagcauuccuuggggcauaaaacaggcuggauuuacuacuggaaugugugucaucauacugaugggccuuuuaacacuuuauugcugcuacagaguagugaaaucacggacuaugauguuuucguuggauaccacuagcugggaauauccagaugucugcagacauuauuucggcuccuuugggcaguggucgagucuccuuuucuccuuggugucucucauuggagcaaugauaguuuauugggugcuuaugucaaauuuucuuuuuaauacuggaaaguuuauuuuuaauuuuauucaucacauuaaugacacagacacuauacugaguaccaauaauagcaacccugugauuuguccaagugccgggaguggaggccauccugacaacagcucuaugauuuucuaugccaaugacacaggagcccaacaguuugaaaaguggugggauaaguccaggacaguccccuuuuaucuuguagggcuccuccucccacugcucaauuucaagucuccuucauuuuuuucaaaauuuaauauccuaggcacagugucuguccuuuauuugauuuuccuugucaccuuuaaggcuguucgcuugggauuucauuuggaauuucauugguuuauaccaacagaauuuuuuguaccagagauaagauuucaguuuccacagcugacuggagugcuuacccuugcuuuuuuuauucauaauuguaucaucacacucuugaagaacaacaagaaacaagaaaacaaugugagggacuugugcauugcuuauaugcuggugacauuaacuuaucucuauauuggaguccugguuuuugcuucauuuccuucaccaccauuauccaaagauuguauugagcagaauuuuuuagacaacuucccuagcagugacacccuguccuucauugcaaggauauuccugcuguuccagaugaugacuguauacccacucuuaggcuaccuggcucguguccagcuuuugggccauaucuucggugacauuuauccuagcauuuuccaugugcugauucuuaaucuaauuauugugggagcuggagugaucauggccuguuucuacccaaacauaggagggaucauaagauauucaggagcagcauguggacuggccuuuguauucauauacccaucucucaucuauauaauuucccuccaccaagaagagcgucugacauggccuaaauuaaucuuccacguuuucaucaucauuuugggcguggcuaaccugauuguucaguuuuuuaugugaaauaccucaacuguuuuuuucaagagcucucaugauauuuugagccuugacaacaguucuauauaaauucacuuguaaaugcugcuguuguguaauucuaaauauuuucuaagauaauuugaaagcaagggaaauaguggccccuuaaugaguauuuuuuuauugggguggggaaaggggcaaaaagaaugaucuuagugucuuuaccuuucucauauuaacucaccucuuuauucuguggucuuuucugaauagaaauguaugcccuaggaagaaaucaugcuggguuuugcuuuuagagauaaaaggugguggauuuauuuugccugcaguaaagauucucagggugucagagcagcauauugucaaauccugcuucuguuuuauguuucaguguauucacuuucauuuucuuacuuacuagaccauuucugcaguuugcccaaaccucuacuguuugggacaguaagccaaauaccucauuuuuaaaaagaaguuuucauggcaucaguguuaauaaaguacauuuuuaacugagucuuaaucucuauuugaagaaaaaguagagacaaaaguaaugucaauguaauccccaggaucaugaaauguauacaaaauaaauaaaguaggagaguuuguugcugucuaaaaaaaaa

SEQ ID NO: 20 - protein, 498 aa, Homo sapiens
  Amino acid sequence of homo sapiens SLC38A9 isoform 4 (NP_001269358.1)
MNKRIHYYSRLTTPADKALIAPDHVVPAPEECYVYSPLGSAYKLQSYTEGYGKNTSLVTIFMIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILGTVSVLYLIFLVTFKAVRLGFHLEFHWFIPTEFFVPEIRFQFPQLTGVLTLAFFIHNCIITLLKNNKKQENNVRDLCIAYMLVTLTYLYIGVLVFASFPSPPLSKDCIEQNFLDNFPSSDTLSFIARIFLLFQMMTVYPLLGYLARVQLLGHIFGDIYPSIFHVLILNLIIVGAGVIMACFYPNIGGIIRYSGAACGLAFVFIYPSLIYIISLHQEERLTWPKLIFHVFIIILGVANLIVQFFM

SEQ ID NO: 21 - DNA, 333 bp, Homo sapiens
  Nucleotide sequence encoding the N-terminal cytoplasmic region of homo sapiens SLC38A9 (Sentor) isoform 1
atggcaaatatgaatagtgattctaggcatcttggcacctctgaggtagatcatgaaagagatcctggacctatgaatatccagtttgagccatcggatctaagatccaaaaggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacact

SEQ ID NO: 22 - protein, 111 aa, Homo sapiens
  Amino acid sequence of the N-terminal cytoplasmic region of homo sapiens SLC38A9 (Sentor) isoform 1
MANMNSDSRHLGTSEVDHERDPGPMNIQFEPSDLRSKRPFCIEPTNIVNVNHVIQRVSDHASAMNKRIHYYSRLTTPADKALIAPDHVVPAPEECYVYSPLGSAYKLQSYT

SEQ ID NO: 23 - RNA, 2631 nt, Homo sapiens
  Nucleotide sequence encoding homo sapiens Deptor NM_022783.2
ucuggggccggacuagcccagcgcgucugccuacccauagggauucccucuccagccaauccagucagagcagcggagcugccccgaacaaagauggcgcgggaagcgucugugagggcagacugauccgagcacccaaacccucggcggacagcggagccagugguagccgcacggcccuaaaaccauggaggagggcggcagcacuggcagugcuggcagugacagcagcaccagcgggaguggcggggcgcagcaaagggagcuggagcgcauggcugaggucuuggucaccggggaacagcuacggcucaggcugcacgaagaaaagguuauuaaagauagacgucaucaucucaagaccuacccaaacuguuuugucgcaaaagaacugauugacuggcugauugaacacaaagaggcuucugacagagagacggcaauuaaacucaugcagaaauuagcagaccggggcauuauucaccaugugugugaugagcauaaggaauucaaggaugucaaacucuucuaccgcuuuagaaaggaugacggcaccuucccauuggauaaugaagugaaggccuuuaugagaggacagaggcuauaugaaaagcugaugagcccugaaaacacacuccugcagcccagggaggaggaaggggucaaguaugagcgcaccuucauggcaucugaauuccuggacuggcugguucaggaaggugaggccaccacgaggaaagaggcagagcagcuuugccaccggcuuauggagcauggcaucauccagcauguguccaacaagcacccauuuguggacagcaaucuucucuaccaguucagaaugaacuuccggcggaggcgaagacugauggagcugcucaaugaaaagucccccuccucccaggaaacucaugacagucccuucugccugaggaagcagagccaugacaaucggaaaucuaccagcuuuaugucagugagccccagcaaggagaucaagaucgugucugcagugaggagaagcagcaugagcagcuguggcagcagcggcuacuucagcagcagccccacccucagcagcagccccccugugcucugcaaccccaaguccgugcugaagagaccugucaccucugaggaacuccuuacucccggggcuccguaugcaaggaagacauucacgauuguuggugacgcgguuggcugggguuuuguggugcgaggaaguaagccaugccacauccaggcuguagaccccaguggcccugcagccgcagcaggaaugaaggucugucaguuugucgucucugucaacgggcucaauguccugcauguagacuaccggaccgugagcaaucugauucugacgggcccacggacgauugucauggaagucauggaggaguuagagugcugagcuccugggccucccagcccuccaguggccugugggugagggaagccagaaugacacaaagcaaugcaaagacaagauugccaugcaaauggaugguuuuggacauacgagucuucuccgcacauacaugucuaaaguugaguuuuauacacugaauguggaagaaccggguaucauaucuuuuuuaaaaaaugucaguguagaaaacauuugggaaaccauuuuccuacaugauagaacugccuuacuagauuucuauuuguagcucucauucauuguuuuuuaucuuaguuugcagaaagguguugaaaugcuucucuagcccaaacagcgacaugcuaaaguccccuucuucagagucaauagaguaguuguuaaagguuuuaaauuguacuuucuccaaaauuagcaugcagcuauuuaauagggaaucuagauuucaccaagauucaaaucaaagcaacauuuaaaggaauaagaccuguucacuagcauuuucaaggggguucuaaagcauucaagugcuuaaaagccauaaaaaaugacuucuuaauuccugccuuuagugucaacuuuuaaguuaauacagguuucaauuguggcauuaggaaaaaaaaaaaccuugugaugcuaugguuggggguaguuagggagagacuacaugaaauugugugccccuauuuucuuucugauccuaaaucauuuuguuuuauaaaucagcuauagcaucuuucuagaauuaauccugaauauguugaauguuaaaauagagaaguuuguauauacacauaauuaaaaaucaacccuucuggcaagauuucacuuugaaggugucuguuuuuaagggaaggcuaaaacuuugcugauaugugauaaaacuugaacucuaaauugccuccuuagccugaauuuuuacuagcuuucuaguguaacauauucuaaggacaugacaugcugcuuauuuggggggauuauuuuucaccaaaaugaucaucuuguguuguaacuuuucagcucacuuguauauacgcacguuuucauuuuuggucuagaaaauagugcauuuuggggcucagcgugcagaggaaauuccucaaagugccaaauuaggcagucaggaaauacuaaauucuucauuccaaauaauggaucugaaugaugacguuaauuuuuuaucaugcauuaacaaaauaaaauaugagaauuguaccaca

SEQ ID NO: 24 - DNA, 1230 bp, Homo sapiens
  Nucleotide sequence encoding homo sapiens Deptor NM_022783.2
atggaggagggcggcagcactggcagtgctggcagtgacagcagcaccagcgggagtggcggggcgcagcaaagggagctggagcgcatggctgaggtcttggtcaccggggaacagctacggctcaggctgcacgaagaaaaggttattaaagatagacgtcatcatctcaagacctacccaaactgttttgtcgcaaaagaactgattgactggctgattgaacacaaagaggcttctgacagagagacggcaattaaactcatgcagaaattagcagaccggggcattattcaccatgtgtgtgatgagcataaggaattcaaggatgtcaaactcttctaccgctttagaaaggatgacggcaccttcccattggataatgaagtgaaggcctttatgagaggacagaggctatatgaaaagctgatgagccctgaaaacacactcctgcagcccagggaggaggaaggggtcaagtatgagcgcaccttcatggcatctgaattcctggactggctggttcaggaaggtgaggccaccacgaggaaagaggcagagcagctttgccaccggcttatggagcatggcatcatccagcatgtgtccaacaagcacccatttgtggacagcaatcttctctaccagttcagaatgaacttccggcggaggcgaagactgatggagctgctcaatgaaaagtccccctcctcccaggaaactcatgacagtcccttctgcctgaggaagcagagccatgacaatcggaaatctaccagctttatgtcagtgagccccagcaaggagatcaagatcgtgtctgcagtgaggagaagcagcatgagcagctgtggcagcagcggctacttcagcagcagccccaccctcagcagcagcccccctgtgctctgcaaccccaagtccgtgctgaagagacctgtcacctctgaggaactccttactcccggggctccgtatgcaaggaagacattcacgattgttggtgacgcggttggctggggttttgtggtgcgaggaagtaagccatgccacatccaggctgtagaccccagtggccctgcagccgcagcaggaatgaaggtctgtcagtttgtcgtctctgtcaacgggctcaatgtcctgcatgtagactaccggaccgtgagcaatctgattctgacgggcccacggacgattgtcatggaagtcatggaggagttagagtgctga

SEQ ID NO: 25 - protein, 409 aa, Homo sapiens
  Amino acid sequence of homo sapiens Deptor NM_022783.2
MEEGGSTGSAGSDSSTSGSGGAQQRELERMAEVLVTGEQLRLRLHEEKVIKDRRHHLKTYPNCFVAKELIDWLIEHKEASDRETAIKLMQKLADRGIIHHVCDEHKEFKDVKLFYRFRKDDGTFPLDNEVKAFMRGQRLYEKLMSPENTLLQPREEEGVKYERTFMASEFLDWLVQEGEATTRKEAEQLCHRLMEHGIIQHVSNKHPFVDSNLLYQFRMNFRRRRRLMELLNEKSPSSQETHDSPFCLRKQSHDNRKSTSFMSVSPSKEIKIVSAVRRSSMSSCGSSGYFSSSPTLSSSPPVLCNPKSVLKRPVTSEELLTPGAPYARKTFTIVGDAVGWGFVVRGSKPCHIQAVDPSGPAAAAGMKVCQFVVSVNGLNVLHVDYRTVSNLILTGPRTIVMEVMEELEC

SEQ ID NO: 26 - RNA, 1901 nt, Homo sapiens
  Nucleotide sequence encoding homo sapiens mLST8 NM_001199173.1
guagugggucagggagggcaccggaacagcgcgacccgacagcccgggaaacggacagccgugucagaagggcaggugccagugggcaggaggccggagagaaagccgcagcuuccuuccaccucgcgccgggcccgcggccgcgcacggggaccgcuccgaguuccucccggcgggaaccccccgccccagaacuuuggucucgucccacccacccccgcccgcgccauggucucgcccuagggagccaucgauaacucuacgcucggccucgaucgacucgcuccggcuccccucgccguccuggacacggcggagugcggagccgcccguaagcucuagggcccgugcaggccacaccaugaacaccuccccaggcacggugggcagugacccggucauccuggccacugcaggcuacgaccacaccgugcgcuucuggcaggcccacagcggcaucugcacccggacggugcagcaccaggacucccaggugaaugccuuggaggucacaccggaccgcagcaugauugcugcugcagguuaccagcacauccgcauguaugaucucaacuccaauaacccuaaccccaucaucagcuacgacggcgucaacaagaacaucgcgucugugggcuuccacgaagacggccgcuggauguacacgggcggcgaggacugcacagccaggaucugggaccucaggucccggaaccugcagugccagcggaucuuccaggugaacgcacccauuaacugcgugugccugcaccccaaccaggcagagcucaucgugggugaccagagcggggcuauccacaucugggacuugaaaacagaccacaacgagcagcugaucccugagcccgaggucuccaucacguccgcccacaucgaucccgacgccagcuacauggcagcugucaauagcaccggaaacugcuaugucuggaaucugacggggggcauuggugacgaggugacccagcucauccccaagacuaagaucccugcccacacgcgcuacgcccugcagugucgcuucagccccgacuccacgcuccucgccaccugcucggcugaucagacgugcaagaucuggaggacguccaacuucucccugaugacggagcugagcaucaagagcggcaaccccggggaguccucccgcggcuggauguggggcugcgccuucucgggggacucccaguacaucgucacugcuuccucggacaaccuggcccggcucugguguguggagacuggagagaucaagagagaguauggcggccaccagaaggcuguugucugccuggccuucaaugacagugugcugggcuagccugugaccccucgggacugccuggugcaggugguggcagcuggagggacccaugcagcacccaggucagagcagacccuccccugccggccugcgccagcuggaccugauggcccccuguggcgccuugaccugcugggccaggcugcccugggacucucagcccccaguugcuuauccagaugugacagagcucgacccaagccaggcugcacacuccuggacugggcuagccugcacugccugggaaagucggccgagggcccaaagcugcugaggggucugaggcuggugcccacccccaagcuaguguguucucugccccucccugcccgcguuucagggccucgguccauagagaacaccaccaccauggccagguggaaggguuuauuagucccugccagcagcuguccucccuggugcagguggccuggccagcccacuggauuggggacgggccaggcugggccaggucgggggcucagucugggagguaauaaaagcagaccgacacgcagauguugcucgggaagcagaaaaaaaaaaaaaaaaaaaa

SEQ ID NO: 27 - DNA, 981 bp, Homo sapiens
  Nucleotide sequence encoding homo sapiens mLST8 NM_001199173.1
atgaacacctccccaggcacggtgggcagtgacccggtcatcctggccactgcaggctacgaccacaccgtgcgcttctggcaggcccacagcggcatctgcacccggacggtgcagcaccaggactcccaggtgaatgccttggaggtcacaccggaccgcagcatgattgctgctgcaggttaccagcacatccgcatgtatgatctcaactccaataaccctaaccccatcatcagctacgacggcgtcaacaagaacatcgcgtctgtgggcttccacgaagacggccgctggatgtacacgggcggcgaggactgcacagccaggatctgggacctcaggtcccggaacctgcagtgccagcggatcttccaggtgaacgcacccattaactgcgtgtgcctgcaccccaaccaggcagagctcatcgtgggtgaccagagcggggctatccacatctgggacttgaaaacagaccacaacgagcagctgatccctgagcccgaggtctccatcacgtccgcccacatcgatcccgacgccagctacatggcagctgtcaatagcaccggaaactgctatgtctggaatctgacggggggcattggtgacgaggtgacccagctcatccccaagactaagatccctgcccacacgcgctacgccctgcagtgtcgcttcagccccgactccacgctcctcgccacctgctcggctgatcagacgtgcaagatctggaggacgtccaacttctccctgatgacggagctgagcatcaagagcggcaaccccggggagtcctcccgcggctggatgtggggctgcgccttctcgggggactcccagtacatcgtcactgcttcctcggacaacctggcccggctctggtgtgtggagactggagagatcaagagagagtatggcggccaccagaaggctgttgtctgcctggccttcaatgacagtgtgctgggctag

SEQ ID NO: 28 - protein, 326 aa, Homo sapiens
  Amino acid sequence of homo sapiens mLST8 NM_001199173.1
MNTSPGTVGSDPVILATAGYDHTVRFWQAHSGICTRTVQHQDSQVNALEVTPDRSMIAAAGYQHIRMYDLNSNNPNPIISYDGVNKNIASVGFHEDGRWMYTGGEDCTARIWDLRSRNLQCQRIFQVNAPINCVCLHPNQAELIVGDQSGAIHIWDLKTDHNEQLIPEPEVSITSAHIDPDASYMAAVNSTGNCYVWNLTGGIGDEVTQLIPKTKIPAHTRYALQCRFSPDSTLLATCSADQTCKIWRTSNFSLMTELSIKSGNPGESSRGWMWGCAFSGDSQYIVTASSDNLARLWCVETGEIKREYGGHQKAVVCLAFNDSVLG

SEQ ID NO: 29 - RNA, 1740 nt, Homo sapiens
  Nucleotide sequence encoding homo sapiens mLST8 NM_001199174.1
cuugcgcacgcgcagcccgccccugcggcagaguggcgcccgcgcgugacuccccccugccggcugcggaggugggggggggacggcgcccccgccgugugcguggggcggggauggagcacgcgcccuggagccccggagccagaugcucugaccuuugaccccugccguucagcucuagggcccgugcaggccacaccaugaacaccuccccaggcacggugggcagugacccggucauccuggccacugcaggcuacgaccacaccgugcgcuucuggcaggcccacagcggcaucugcacccggacggugcagcaccaggacucccaggugaaugccuuggaggucacaccggaccgcagcaugauugcugcugcagguuaccagcacauccgcauguaugaucucaacuccaauaacccuaaccccaucaucagcuacgacggcgucaacaagaacaucgcgucugugggcuuccacgaagacggccgcuggauguacacgggcggcgaggacugcacagccaggaucugggaccucaggucccggaaccugcagugccagcggaucuuccaggugaacgcacccauuaacugcgugugccugcaccccaaccaggcagagcucaucgugggugaccagagcggggcuauccacaucugggacuugaaaacagaccacaacgagcagcugaucccugagcccgaggucuccaucacguccgcccacaucgaucccgacgccagcuacauggcagcugucaauagcaccggaaacugcuaugucuggaaucugacggggggcauuggugacgaggugacccagcucauccccaagacuaagaucccugcccacacgcgcuacgcccugcagugucgcuucagccccgacuccacgcuccucgccaccugcucggcugaucagacgugcaagaucuggaggacguccaacuucucccugaugacggagcugagcaucaagagcggcaaccccggggaguccucccgcggcuggauguggggcugcgccuucucgggggacucccaguacaucgucacugcuuccucggacaaccuggcccggcucugguguguggagacuggagagaucaagagagaguauggcggccaccagaaggcuguugucugccuggccuucaaugacagugugcugggcuagccugugaccccucgggacugccuggugcaggugguggcagcuggagggacccaugcagcacccaggucagagcagacccuccccugccggccugcgccagcuggaccugauggcccccuguggcgccuugaccugcugggccaggcugcccugggacucucagcccccaguugcuuauccagaugugacagagcucgacccaagccaggcugcacacuccuggacugggcuagccugcacugccugggaaagucggccgagggcccaaagcugcugaggggucugaggcuggugcccacccccaagcuaguguguucucugccccucccugcccgcguuucagggccucgguccauagagaacaccaccaccauggccagguggaaggguuuauuagucccugccagcagcuguccucccuggugcagguggccuggccagcccacuggauuggggacgggccaggcugggccaggucgggggcucagucugggagguaauaaaagcagaccgacacgcagauguugcucgggaagcagaaaaaaaaaaaaaaaaaaaa

SEQ ID NO: 30 - DNA, 981 bp, Homo sapiens; identical to SEQ ID NO: 27
  Nucleotide sequence encoding homo sapiens mLST8 NM_001199174.1

SEQ ID NO: 31 - protein, 326 aa, Homo sapiens; identical to SEQ ID NO: 28
  Amino acid sequence of homo sapiens mLST8 NM_001199174.1

SEQ ID NO: 32 - RNA, 1928 nt, Homo sapiens
  Nucleotide sequence encoding homo sapiens mLST8 NM_001199175.1
guagugggucagggagggcaccggaacagcgcgacccgacagcccgggaaacggacagccgugucagaagggcaggugccagugggcaggaggccggagagaaagccgcagcuuccuuccaccucgcgccgggcccgcggccgcgcacggggaccgcuccgaguuccucccggcgggaaccccccgccccagaacuuuggucucgucccacccacccccgcccgcgccauggucucgcccuagggagccaucgauaacucuacgcucggccucgaucgacucgcuccggcuccccucgccguccuggacacggcggagugcggagccgcccguaagaugcucugaccuuugaccccugccguucagcucuagggcccgugcaggccacaccaugaacaccuccccaggcacggugggcagugacccggucauccuggccacugcaggcuacgaccacaccgugcgcuucuggcaggcccacagcggcaucugcacccggacggugcagcaccaggacuccgugaaugccuuggaggucacaccggaccgcagcaugauugcugcugcagguuaccagcacauccgcauguaugaucucaacuccaauaacccuaaccccaucaucagcuacgacggcgucaacaagaacaucgcgucugugggcuuccacgaagacggccgcuggauguacacgggcggcgaggacugcacagccaggaucugggaccucaggucccggaaccugcagugccagcggaucuuccaggugaacgcacccauuaacugcgugugccugcaccccaaccaggcagagcucaucgugggugaccagagcggggcuauccacaucugggacuugaaaacagaccacaacgagcagcugaucccugagcccgaggucuccaucacguccgcccacaucgaucccgacgccagcuacauggcagcugucaauagcaccggaaacugcuaugucuggaaucugacggggggcauuggugacgaggugacccagcucauccccaagacuaagaucccugcccacacgcgcuacgcccugcagugucgcuucagccccgacuccacgcuccucgccaccugcucggcugaucagacgugcaagaucuggaggacguccaacuucucccugaugacggagcugagcaucaagagcggcaaccccggggaguccucccgcggcuggauguggggcugcgccuucucgggggacucccaguacaucgucacugcuuccucggacaaccuggcccggcucugguguguggagacuggagagaucaagagagaguauggcggccaccagaaggcuguugucugccuggccuucaaugacagugugcugggcuagccugugaccccucgggacugccuggugcaggugguggcagcuggagggacccaugcagcacccaggucagagcagacccuccccugccggccugcgccagcuggaccugauggcccccuguggcgccuugaccugcugggccaggcugcccugggacucucagcccccaguugcuuauccagaugugacagagcucgacccaagccaggcugcacacuccuggacugggcuagccugcacugccugggaaagucggccgagggcccaaagcugcugaggggucugaggcuggugcccacccccaagcuaguguguucucugccccucccugcccgcguuucagggccucgguccauagagaacaccaccaccauggccagguggaaggguuuauuagucccugccagcagcuguccucccuggugcagguggccuggccagcccacuggauuggggacgggccaggcugggccaggucgggggcucagucugggagguaauaaaagcagaccgacacgcagauguugcucgggaagcagaaaaaaaaaaaaaaaaaaaa

SEQ ID NO: 33 - DNA, 978 bp, Homo sapiens
  Nucleotide sequence encoding homo sapiens mLST8 NM_001199175.1
atgaacacctccccaggcacggtgggcagtgacccggtcatcctggccactgcaggctacgaccacaccgtgcgcttctggcaggcccacagcggcatctgcacccggacggtgcagcaccaggactccgtgaatgccttggaggtcacaccggaccgcagcatgattgctgctgcaggttaccagcacatccgcatgtatgatctcaactccaataaccctaaccccatcatcagctacgacggcgtcaacaagaacatcgcgtctgtgggcttccacgaagacggccgctggatgtacacgggcggcgaggactgcacagccaggatctgggacctcaggtcccggaacctgcagtgccagcggatcttccaggtgaacgcacccattaactgcgtgtgcctgcaccccaaccaggcagagctcatcgtgggtgaccagagcggggctatccacatctgggacttgaaaacagaccacaacgagcagctgatccctgagcccgaggtctccatcacgtccgcccacatcgatcccgacgccagctacatggcagctgtcaatagcaccggaaactgctatgtctggaatctgacggggggcattggtgacgaggtgacccagctcatccccaagactaagatccctgcccacacgcgctacgccctgcagtgtcgcttcagccccgactccacgctcctcgccacctgctcggctgatcagacgtgcaagatctggaggacgtccaacttctccctgatgacggagctgagcatcaagagcggcaaccccggggagtcctcccgcggctggatgtggggctgcgccttctcgggggactcccagtacatcgtcactgcttcctcggacaacctggcccggctctggtgtgtggagactggagagatcaagagagagtatggcggccaccagaaggctgttgtctgcctggccttcaatgacagtgtgctgggctag

SEQ ID NO: 34 - protein, 325 aa, Homo sapiens
  Amino acid sequence of homo sapiens mLST8 NM_001199175.1
MNTSPGTVGSDPVILATAGYDHTVRFWQAHSGICTRTVQHQDSVNALEVTPDRSMIAAAGYQHIRMYDLNSNNPNPIISYDGVNKNIASVGFHEDGRWMYTGGEDCTARIWDLRSRNLQCQRIFQVNAPINCVCLHPNQAELIVGDQSGAIHIWDLKTDHNEQLIPEPEVSITSAHIDPDASYMAAVNSTGNCYVWNLTGGIGDEVTQLIPKTKIPAHTRYALQCRFSPDSTLLATCSADQTCKIWRTSNFSLMTELSIKSGNPGESSRGWMWGCAFSGDSQYIVTASSDNLARLWCVETGEIKREYGGHQKAVVCLAFNDSVLG

SEQ ID NO: 35 - RNA, 1931 nt, Homo sapiens
  Nucleotide sequence encoding homo sapiens mLST8 NM_022372.4
guagugggucagggagggcaccggaacagcgcgacccgacagcccgggaaacggacagccgugucagaagggcaggugccagugggcaggaggccggagagaaagccgcagcuuccuuccaccucgcgccgggcccgcggccgcgcacggggaccgcuccgaguuccucccggcgggaaccccccgccccagaacuuuggucucgucccacccacccccgcccgcgccauggucucgcccuagggagccaucgauaacucuacgcucggccucgaucgacucgcuccggcuccccucgccguccuggacacggcggagugcggagccgcccguaagaugcucugaccuuugaccccugccguucagcucuagggcccgugcaggccacaccaugaacaccuccccaggcacggugggcagugacccggucauccuggccacugcaggcuacgaccacaccgugcgcuucuggcaggcccacagcggcaucugcacccggacggugcagcaccaggacucccaggugaaugccuuggaggucacaccggaccgcagcaugauugcugcugcagguuaccagcacauccgcauguaugaucucaacuccaauaacccuaaccccaucaucagcuacgacggcgucaacaagaacaucgcgucugugggcuuccacgaagacggccgcuggauguacacgggcggcgaggacugcacagccaggaucugggaccucaggucccggaaccugcagugccagcggaucuuccaggugaacgcacccauuaacugcgugugccugcaccccaaccaggcagagcucaucgugggugaccagagcggggcuauccacaucugggacuugaaaacagaccacaacgagcagcugaucccugagcccgaggucuccaucacguccgcccacaucgaucccgacgccagcuacauggcagcugucaauagcaccggaaacugcuaugucuggaaucugacggggggcauuggugacgaggugacccagcucauccccaagacuaagaucccugcccacacgcgcuacgcccugcagugucgcuucagccccgacuccacgcuccucgccaccugcucggcugaucagacgugcaagaucuggaggacguccaacuucucccugaugacggagcugagcaucaagagcggcaaccccggggaguccucccgcggcuggauguggggcugcgccuucucgggggacucccaguacaucgucacugcuuccucggacaaccuggcccggcucugguguguggagacuggagagaucaagagagaguauggcggccaccagaaggcuguugucugccuggccuucaaugacagugugcugggcuagccugugaccccucgggacugccuggugcaggugguggcagcuggagggacccaugcagcacccaggucagagcagacccuccccugccggccugcgccagcuggaccugauggcccccuguggcgccuugaccugcugggccaggcugcccugggacucucagcccccaguugcuuauccagaugugacagagcucgacccaagccaggcugcacacuccuggacugggcuagccugcacugccugggaaagucggccgagggcccaaagcugcugaggggucugaggcuggugcccacccccaagcuaguguguucucugccccucccugcccgcguuucagggccucgguccauagagaacaccaccaccauggccagguggaaggguuuauuagucccugccagcagcuguccucccuggugcagguggccuggccagcccacuggauuggggacgggccaggcugggccaggucgggggcucagucugggagguaauaaaagcagaccgacacgcagauguugcucgggaagcagaaaaaaaaaaaaaaaaaaaa

SEQ ID NO: 36 - DNA, 981 bp, Homo sapiens; identical to SEQ ID NO: 27
  Nucleotide sequence encoding homo sapiens mLST8 NM_022372.4

SEQ ID NO: 37 - protein, 326 aa, Homo sapiens; identical to SEQ ID NO: 28
  Amino acid sequence of homo sapiens mLST8 NM_022372.4

SEQ ID NO: 38 - RNA, 8733 nt, Homo sapiens
  Nucleotide sequence encoding homo sapiens mTOR NM_004958.3
gcucccggcuuagaggacagcggggaaggcgggcgguggggcagggggccugaagcggcgguaccggugcuggcggcggcagcugaggccuuggccgaagccgcgcgaaccucagggcaagaugcuuggaaccggaccugccgccgccaccaccgcugccaccacaucuagcaaugugagcguccugcagcaguuugccaguggccuaaagagccggaaugaggaaaccagggccaaagccgccaaggagcuccagcacuaugucaccauggaacuccgagagaugagucaagaggagucuacucgcuucuaugaccaacugaaccaucacauuuuugaauugguuuccagcucagaugccaaugagaggaaagguggcaucuuggccauagcuagccucauaggaguggaaggugggaaugccacccgaauuggcagauuugccaacuaucuucggaaccuccuccccuccaaugacccaguugucauggaaauggcauccaaggccauuggccgucuugccauggcaggggacacuuuuaccgcugaguacguggaauuugaggugaagcgagcccuggaauggcugggugcugaccgcaaugagggccggagacaugcagcuguccugguucuccgugagcuggccaucagcgucccuaccuucuucuuccagcaagugcaacccuucuuugacaacauuuuuguggccgugugggaccccaaacaggccauccgugagggagcuguagccgcccuucgugccugucugauucucacaacccagcgugagccgaaggagaugcagaagccucagugguacaggcacacauuugaagaagcagagaagggauuugaugagaccuuggccaaagagaagggcaugaaucgggaugaucggauccauggagccuuguugauccuuaacgagcugguccgaaucagcagcauggagggagagcgucugagagaagaaauggaagaaaucacacagcagcagcugguacacgacaaguacugcaaagaucucaugggcuucggaacaaaaccucgucacauuacccccuucaccaguuuccaggcuguacagccccagcagucaaaugccuugguggggcugcugggguacagcucucaccaaggccucaugggauuugggaccucccccaguccagcuaaguccacccugguggagagccgguguugcagagacuugauggaggagaaauuugaucaggugugccagugggugcugaaaugcaggaauagcaagaacucgcugauccaaaugacaauccuuaauuuguugccccgcuuggcugcauuccgaccuucugccuucacagauacccaguaucuccaagauaccaugaaccauguccuaagcugugucaagaaggagaaggaacguacagcggccuuccaagcccuggggcuacuuucuguggcugugaggucugaguuuaaggucuauuugccucgcgugcuggacaucauccgagcggcccugcccccaaaggacuucgcccauaagaggcagaaggcaaugcagguggaugccacagucuucacuugcaucagcaugcuggcucgagcaauggggccaggcauccagcaggauaucaaggagcugcuggagcccaugcuggcagugggacuaagcccugcccucacugcagugcucuacgaccugagccgucagauuccacagcuaaagaaggacauucaagaugggcuacugaaaaugcugucccugguccuuaugcacaaaccccuucgccacccaggcaugcccaagggccuggcccaucagcuggccucuccuggccucacgacccucccugaggccagcgaugugggcagcaucacucuugcccuccgaacgcuuggcagcuuugaauuugaaggccacucucugacccaauuuguucgccacugugcggaucauuuccugaacagugagcacaaggagauccgcauggaggcugcccgcaccugcucccgccugcucacacccuccauccaccucaucaguggccaugcucaugugguuagccagaccgcagugcaagugguggcagaugugcuuagcaaacugcucguaguugggauaacagauccugacccugacauucgcuacugugucuuggcgucccuggacgagcgcuuugaugcacaccuggcccaggcggagaacuugcaggccuuguuuguggcucugaaugaccagguguuugagauccgggagcuggccaucugcacugugggccgacucaguagcaugaacccugccuuugucaugccuuuccugcgcaagaugcucauccagauuuugacagaguuggagcacagugggauuggaagaaucaaagagcagagugcccgcaugcuggggcaccuggucuccaaugccccccgacucauccgccccuacauggagccuauucugaaggcauuaauuuugaaacugaaagauccagacccugauccaaacccaggugugaucaauaauguccuggcaacaauaggagaauuggcacagguuaguggccuggaaaugaggaaauggguugaugaacuuuuuauuaucaucauggacaugcuccaggauuccucuuuguuggccaaaaggcagguggcucuguggacccugggacaguugguggccagcacuggcuauguaguagagcccuacaggaaguacccuacuuugcuugaggugcuacugaauuuucugaagacugagcagaaccaggguacacgcagagaggccauccguguguuagggcuuuuaggggcuuuggauccuuacaagcacaaagugaacauuggcaugauagaccagucccgggaugccucugcugucagccugucagaauccaagucaagucaggauuccucugacuauagcacuagugaaaugcuggucaacaugggaaacuugccucuggaugaguucuacccagcuguguccaugguggcccugaugcggaucuuccgagaccagucacucucucaucaucacaccaugguuguccaggccaucaccuucaucuucaagucccugggacucaaaugugugcaguuccugccccaggucaugcccacguuccuuaacgucauucgagucugugauggggccauccgggaauuuuuguuccagcagcugggaauguugguguccuuugugaagagccacaucagaccuuauauggaugaaauagucacccucaugagagaauucugggucaugaacaccucaauucagagcacgaucauucuucucauugagcaaauugugguagcucuugggggugaauuuaagcucuaccugccccagcugaucccacacaugcugcgugucuucaugcaugacaacagcccaggccgcauugucucuaucaaguuacuggcugcaauccagcuguuuggcgccaaccuggaugacuaccugcauuuacugcugccuccuauuguuaaguuguuugaugccccugaagcuccacugccaucucgaaaggcagcgcuagagacuguggaccgccugacggagucccuggauuucacugacuaugccucccggaucauucacccuauuguucgaacacuggaccagagcccagaacugcgcuccacagccauggacacgcugucuucacuuguuuuucagcuggggaagaaguaccaaauuuucauuccaauggugaauaaaguucuggugcgacaccgaaucaaucaucagcgcuaugaugugcucaucugcagaauugucaagggauacacacuugcugaugaagaggaggauccuuugauuuaccagcaucggaugcuuaggaguggccaaggggaugcauuggcuaguggaccaguggaaacaggacccaugaagaaacugcacgucagcaccaucaaccuccaaaaggccuggggcgcugccaggagggucuccaaagaugacuggcuggaauggcugagacggcugagccuggagcugcugaaggacucaucaucgcccucccugcgcuccugcugggcccuggcacaggccuacaacccgauggccagggaucucuucaaugcugcauuuguguccugcuggucugaacugaaugaagaucaacaggaugagcucaucagaagcaucgaguuggcccucaccucacaagacaucgcugaagucacacagacccucuuaaacuuggcugaauucauggaacacagugacaagggcccccugccacugagagaugacaauggcauuguucugcugggugagagagcugccaagugccgagcauaugccaaagcacuacacuacaaagaacuggaguuccagaaaggccccaccccugccauucuagaaucucucaucagcauuaauaauaagcuacagcagccggaggcagcggccggaguguuagaauaugccaugaaacacuuuggagagcuggagauccaggcuaccugguaugagaaacugcacgagugggaggaugcccuuguggccuaugacaagaaaauggacaccaacaaggacgacccagagcugaugcugggccgcaugcgcugccucgaggccuugggggaauggggucaacuccaccagcagugcugugaaaaguggacccugguuaaugaugagacccaagccaagauggcccggauggcugcugcagcugcaugggguuuaggucagugggacagcauggaagaauacaccuguaugaucccucgggacacccaugauggggcauuuuauagagcugugcuggcacugcaucaggaccucuucuccuuggcacaacagugcauugacaaggccagggaccugcuggaugcugaauuaacugcgauggcaggagagaguuacagucgggcauauggggccaugguuucuugccacaugcuguccgagcuggaggagguuauccaguacaaacuuguccccgagcgacgagagaucauccgccagaucuggugggagagacugcagggcugccagcguaucguagaggacuggcagaaaauccuuauggugcggucccuuguggucagcccucaugaagacaugagaaccuggcucaaguaugcaagccugugcggcaagaguggcaggcuggcucuugcucauaaaacuuuaguguugcuccugggaguugauccgucucggcaacuugaccauccucugccaacaguucacccucaggugaccuaugccuacaugaaaaacauguggaagagugcccgcaagaucgaugccuuccagcacaugcagcauuuuguccagaccaugcagcaacaggcccagcaugccaucgcuacugaggaccagcagcauaagcaggaacugcacaagcucauggcccgaugcuuccugaaacuuggagaguggcagcugaaucuacagggcaucaaugagagcacaauccccaaagugcugcaguacuacagcgccgccacagagcacgaccgcagcugguacaaggccuggcaugcgugggcagugaugaacuucgaagcugugcuacacuacaaacaucagaaccaagcccgcgaugagaagaagaaacugcgucaugccagcggggccaacaucaccaacgccaccacugccgccaccacggccgccacugccaccaccacugccagcaccgagggcagcaacagugagagcgaggccgagagcaccgagaacagccccaccccaucgccgcugcagaagaaggucacugaggaucuguccaaaacccuccugauguacacggugccugccguccagggcuucuuccguuccaucuccuugucacgaggcaacaaccuccaggauacacucagaguucucaccuuaugguuugauuauggucacuggccagaugucaaugaggccuuaguggagggggugaaagccauccagauugauaccuggcuacagguuauaccucagcucauugcaagaauugauacgcccagacccuuggugggacgucucauucaccagcuucucacagacauuggucgguaccacccccaggcccucaucuacccacugacaguggcuucuaagucuaccacgacagcccggcacaaugcagccaacaagauucugaagaacaugugugagcacagcaacacccugguccagcaggccaugauggugagcgaggagcugauccgaguggccauccucuggcaugagauguggcaugaaggccuggaagaggcaucucguuuguacuuuggggaaaggaacgugaaaggcauguuugaggugcuggagcccuugcaugcuaugauggaacggggcccccagacucugaaggaaacauccuuuaaucaggccuauggucgagauuuaauggaggcccaagaguggugcaggaaguacaugaaaucagggaaugucaaggaccucacccaagccugggaccucuauuaucauguguuccgacgaaucucaaagcagcugccucagcucacauccuuagagcugcaauauguuuccccaaaacuucugaugugccgggaccuugaauuggcugugccaggaacauaugaccccaaccagccaaucauucgcauucaguccauagcaccgucuuugcaagucaucacauccaagcagaggccccggaaauugacacuuaugggcagcaacggacaugaguuuguuuuccuucuaaaaggccaugaagaucugcgccaggaugagcgugugaugcagcucuucggccugguuaacacccuucuggccaaugacccaacaucucuucggaaaaaccucagcauccagagauacgcugucaucccuuuaucgaccaacucgggccucauuggcuggguuccccacugugacacacugcacgcccucauccgggacuacagggagaagaagaagauccuucucaacaucgagcaucgcaucauguugcggauggcuccggacuaugaccacuugacucugaugcagaagguggagguguuugagcaugccgucaauaauacagcuggggacgaccuggccaagcugcuguggcugaaaagccccagcuccgaggugugguuugaccgaagaaccaauuauacccguucuuuagcggucaugucaaugguuggguauauuuuaggccugggagauagacacccauccaaccugaugcuggaccgucugagugggaagauccugcacauugacuuuggggacugcuuugagguugcuaugacccgagagaaguuuccagagaagauuccauuuagacuaacaagaauguugaccaaugcuauggagguuacaggccuggauggcaacuacagaaucacaugccacacagugauggaggugcugcgagagcacaaggacagugucauggccgugcuggaagccuuugucuaugaccccuugcugaacuggaggcugauggacacaaauaccaaaggcaacaagcgaucccgaacgaggacggauuccuacucugcuggccagucagucgaaauuuuggacgguguggaacuuggagagccagcccauaagaaaacggggaccacagugccagaaucuauucauucuuucauuggagacgguuuggugaaaccagaggcccuaaauaagaaagcuauccagauuauuaacaggguucgagauaagcucacuggucgggacuucucucaugaugacacuuuggauguuccaacgcaaguugagcugcucaucaaacaagcgacaucccaugaaaaccucugccagugcuauauuggcuggugcccuuucugguaacuggaggcccagaugugcccaucacguuuuuucugaggcuuuuguacuuuaguaaaugcuuccacuaaacugaaaccauggugagaaaguuugacuuuguuaaauauuuugaaauguaaaugaaaagaacuacuguauauuaaaaguugguuugaaccaacuuucuagcugcuguugaagaauauauugucagaaacacaaggcuugauuugguucccaggacagugaaacauaguaauaccacguaaaucaagccauucauuuuggggaacagaagauccauaacuuuagaaauacggguuuugacuuaacucacaagagaacucaucauaaguacuugcugauggaagaaugaccuaguugcuccucucaacauggguacagcaaacucagcacagccaagaagccucaggucguggagaacauggauuaggauccuagacuguaaagacacagaagaugcugaccucaccccugccaccuaucccaagaccucacuggucuguggacagcagcagaaauguuugcaagauaggccaaaaugaguacaaaaggucugucuuccaucagacccagugaugcugcgacucacacgcuucaauucaagaccugaccgcuaguagggagguuuauucagaucgcuggcagccucggcugagcagaugcacagaggggaucacugugcagugggaccacccucacuggccuucugcagcaggguucugggauguuuucaguggucaaaauacucuguuuagagcaagggcucagaaaacagaaauacugucauggaggugcugaacacagggaaggucugguacauauuggaaauuaugagcagaacaaauacucaacuaaaugcacaaaguauaaaguguagccaugucuagacaccauguuguaucagaauaauuuuugugccaauaaaugacaucagaauuuuaaacauauguaaaaaaaaa

SEQ ID NO: 39 - DNA, 7650 bp, Homo sapiens
  Nucleotide sequence encoding homo sapiens mTOR NM_004958.3
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 40 - protein, 2549 aa, Homo sapiens
  Amino acid sequence of homo sapiens mTOR NM_004958.3
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 41 - RNA, 6856 nt, Homo sapiens
  Nucleotide sequence encoding homo sapiens Raptor NM_020761.2
cggugcauucuggguccuggcaauauggcguccuccuugaugggcugaugagaugaguuucacuguagcuccaaaccagagggcaaagcucccaugacccaauaagcccacauugucccuuuccuccgugguuccgugucgcccguuucucaggacucguucucaggcaggagagagccucggggcugaaggccaggaccagccaggccgcgcggaccugagguugaggaaccgggugcaggcgagcacgaugggccggucguggcucugguugcagcagcucagacgagugcgggacccgcagggcugagaguggcuggaggagacccagggcccuuugaacccgaucccuuggccggagaccucagcccagucggcccagugggcgaaccggcaccaagagcggccugccugucuucggaacugcugaggcgguggaggccgagagcagggucaucgugaggccugaagucucuuacgcuuuuggcagcuccccucgcagccccucuggaaacguacagccucaggagcagccaguggcuugggaccugggguggugugugucugcggagcuucuugggcugccccauuuccuagcggcccccaccuccccacuucccgcucagaguuagagauaaggaucucagacuuuugccugaguaagggucuccgcacucuuuauccauuugguuuucgauuucccguuuuuguuucuuauuucaccaauucugguacacgcuaguuuuuaaggcuggagguucucgagcgcuugcugccaaggacucccccacccccucccccacugauggaguccgaaaugcugcaaucgccucuucugggccugggggaggaagaugaggcugaucuuacagacuggaaccuaccuuuggcuuuuaugaaaaagaggcacugugagaaaauugaaggcuccaaauccuuagcucagagcuggaggaugaaggaucggaugaagacagucaguguugccuuaguuuugugccugaauguugguguggacccucccgauguggugaagaccacgcccugugcacgcuuggaaugcuggaucgauccucugucgauggguccucagaaagcucuggaaaccaucggugcaaauuuacagaagcaguacgagaacuggcagccaagggcccgguacaagcagagccuugacccaacuguggaugaagucaagaagcucugcacguccuuacgucgcaacgccaaggaggagcgaguccucuuucacuacaauggccacggggugccccggcccacagucaacggggaggucugggucuucaacaagaacuacacgcaguacaucccucuguccauauaugaccugcagacguggaugggcagcccgucgaucuucgucuacgacugcuccaaugcuggcuugaucgucaaguccuucaagcaguucgcacuacagcgggagcaggagcuggagguagcugcaaucaacccaaaucacccucuugcucagaugccuuugccuccgucgaugaaaaacugcauccagcuggcagccugcgaggccaccgagcugcugcccaugauccccgaccucccggcugaccuauucaccuccugccucaccacccccaucaagaucgcccugcgcugguuuugcaugcagaaaugugucagucuggugccuggcgucacacuggauuugauagaaaagaucccuggccgccugaacgacaggaggacgccccugggugaacugaacuggaucuucacagccaucacagacaccaucgcguggaacgugcucccccgggaucucuuccaaaagcucuucagacaggacuugcugguggcuagucuguuucgaaauuuuuuauuggcggaaaggauuaugaggucguauaacugcacucccgucagcagcccgcgucugccgcccacguacaugcacgccauguggcaagccugggaccuggcuguugacaucugucugucucagcugccgacgaucaucgaggaaggcacugcguuucggcacagcccguucuucgccgagcagcugaccgcauuccagguguggcucaccaugggcguggagaaccgaaacccacccgaacagcugcccaucguccugcaggugcuguuaagccaagugcaccggcugagagcauuggacuugcuuggaagauuuuuggaccugggucccugggcagugagccuggccuugucugucggcaucuuccccuacgugcugaagcugcuccagagcucggcccgagagcugcggccacuucucguuuucaucugggccaagauccucgcaguggacagcucgugccaagcggaccucgugaaggacaacggccacaaguacuuccugucgguccuggcggaccccuacaugccagcugaacaccggaccaugacggcuuucauucucgccgugaucgucaacagcuaucacacggggcaggaagccugccuucagggaaaccucauugccaucugccuggagcagcucaacgacccgcaccccuugcugcgccaguggguggccaucugccucggcaggaucuggcagaacuucgacucggcgagguggugcggcgugagggacagcgcucaugagaagcucuacagccuccucuccgaccccauucccgagguccgcugcgcagcggucuucgcccuuggcacguucgugggcaacucugcagagaggacggaccacuccaccaccaucgaccacaacguggccaugaugcuggcccagcuggucagcgacgggagccccaugguccggaaggagcuggugguggcucugagucaucuugugguucaguaugaaagcaauuucugcaccguggcccugcaguucauagaagaggaaaagaacuacgccuugccuucuccagcaaccacagagggagggaguuugaccccagugcgagacagcccgugcacccccagacuucguucugugagcuccuauggaaacauccgugcugucgccacagccaggagccucaacaaaucuuugcagaaccugaguuugacagaggaaucugguggcgcgguggcguucucccccggaaaccucagcaccagcagcagcgccagcagcacccugggcagccccgagaaugaggagcauauccuguccuucgagaccaucgacaagaugcgccgcgccagcuccuacuccucccucaacucccucaucggaguuuccuuuaacaguguuuacacucagauuuggagaguccugcugcaccuggcugcugaccccuauccagaggucucggacguggccaugaaaguacucaacagcaucgccuacaaggccaccgugaacgcccggccgcagcgcguccuggacaccuccucccucacgcagucggcccccgccagccccaccaacaagggcgugcacauccaccaggcggggggcuccccuccggcguccagcaccagcagcuccagccugaccaacgauguggccaagcagccggucagccgagacuugccuucuggccggccgggcaccacaggccccgcuggggcgcaguacaccccucacucccaccaguucccccggacacggaagauguucgacaagggcccagagcagacugcggacgacgcggacgaugcugcuggacacaaaaguuucaucuccgccacggugcagacgggguucugcgacuggagcgcccgcuauuuugcccagcccgucaugaagaucccagaagagcacgaccuggagagucagauccgcaaggagcgggaguggcgguuccugcgaaacagccgugucaggaggcaggcccagcaagucauucagaagggcauuacgagauuggacgaccaaauauuucugaacaggaaccccggcguccccucuguggugaaauuccaccccuucacgccgugcaucgccguagccgacaaggacagcaucugcuuuugggacugggagaaaggggagaagcuggauuauuuccacaaugggaacccucgguacacgagggucacugccauggaguaucugaacggccaggacugcucgcuucugcugacggccacagacgauggugccaucagggucuggaagaauuuugcugauuuggaaaagaacccagagauggugaccgcguggcaggggcucucggacaugcugccaacgacgcgaggagcugggauggugguggacugggagcaggagaccggccuccucaugagcucaggagacgugcggaucguccggaucugggacacagaccgugagaugaaggugcaggacaucccuacgggcgcagacagcugugugacgagucuguccugugauucccaccgcucacucaucguggcuggccucggugacggcuccauccgcgucuacgacagaaggauggcacucagcgaaugccgcgucaugacguaccgggagcacacagccuggguggugaaggccucccugcagaagcgucccgacggccacaucgugagugugagcgucaauggagaugugcgcaucuuugauccccggaugccugagucgguaaaugugcuucagaucgugaaggggcugacggcccuggacauccacccccaggcggaccugaucgcauguggcuccgucaaucaguucaccgccaucuacaacagcagcggagagcucaucaacaacaucaaguacuacgacggcuucaugggccagcgggucggcgccaucagcugccuggccuuccacccgcacuggccucaccuggccgugggaagcaacgacuacuacaucuccguguacucgguggagaagcgugucagauagcggcgugacccgggcccaccaggccacggccgccugcuguacauagugaagcugucacucgccggggcacggggcgucggcugcugcggccccgcagugugaacguuggcugcugccuuagcugcugaugacggcaggagggcccugcuacucgcuuuugucugucuucgcugucgugucuggaaugucagggaaggggagggcucggguugacgguggcuucccacugagcaccagcauccaggugcacccccgcggccacggcgccucugucccucuccuguucuguguuucucugagacgcugaaaggggaaacaccucacuuuauuuccauguaaucagagcauuagcugcagaaaaaccccccgacagagcccuggcggagaggcaggcgcuggggcuccuacgggucccuggggcagcuguccccaucaggccaagagcgagcgagaggcgcugccccagccaggcccaccaccucucacagucagugcacgcaagcagggacauuuccuagccagcugggggacacuggaaauucgggaaaccaagagagaggaagaaggagacgccccuccaacuggcgggugugaaggaagccgcccagggguccgggcuguccuuggccgcuggcagcaucacugagcaggaagcgcacagcccacccuccccgcaccuccaggucucuggacuccaguuuuggccccucucacacagagcugucagcaggggccgcuguggcggugcacaggggaggcagguccuuggcgagguagccccugccuuaauccacggggcuccuuucccuccgaagggcugcucuuccccacaggcgcggggacagcagcccgaccuguggucuccaugccugugcccucacacagguguagcacacgcaugugcagauggcaccacggccggcaccugggggcacacacaugcaggcggcguggucucccugcucuguccccacacguuccucacauacaggcaagaggcacugccgggucccggacggcuccgggugacaccagccccgucuccagccuugagccgcccaugcugaugcgaccucggcugacagcugggccuguggugcagacaggagcuguguggacagucccgcccaggaggggccgcagggcguguaugagcaguuuugcaaacagaacacaaccacaaugaugguauuuugaaaaguguucuuuccguguucgucgggaaucaggauuauugagaggugaaggagccagguggcuucauucuggcggugagaggcccacgaccacgggagugagagcugguguggcgaggcccggcucuccugcgguguggcugguggccugccguggccaagagcaucuucuggguggauggaacccugccuggucacauuuggccagagacacaccuggcccucagggggcugagcuggagacugagcuggggcuggccgggacgugacaaggcaggacagaggcggccccuccgcugcuccuuuuuggaaugcgagcucccaccagaagaagguuccggcacgaaucccauccccacgucugggccgagaaagcagcccggguccggaagguguagagagucccggccucacucagcucacagggcgugccaggcggcaacaccagaaucuuccagaagcccagcuccacccgcacacgcagcuucccauccaguccuucaacucaauucuuacccaacacgcguuucuguuuguuuugagacaaaaucaccaccugucaaaaggcagguggcuccagaggggucaagacccccccccgcccccgcuccacccuggagcccacccccaugggcaccgcgugccgccugcacgugggcugucuucacaggucugaugugaaaauucaaucacgacguuaaccggcucgagagagcgccggccuagaggcucauuaucuauuuauuuuaccaaacgcgaauugagacggacuuugacaaaacacgaaaugguaaugugaagcuaagagcagagagugaccaacaguaaacaacacgcgcagacuccg

SEQ ID NO: 42 - DNA, 4008 bp, Homo sapiens
  Nucleotide sequence encoding homo sapiens Raptor NM_020761.2
atggagtccgaaatgctgcaatcgcctcttctgggcctgggggaggaagatgaggctgatcttacagactggaacctacctttggcttttatgaaaaagaggcactgtgagaaaattgaaggctccaaatccttagctcagagctggaggatgaaggatcggatgaagacagtcagtgttgccttagttttgtgcctgaatgttggtgtggaccctcccgatgtggtgaagaccacgccctgtgcacgcttggaatgctggatcgatcctctgtcgatgggtcctcagaaagctctggaaaccatcggtgcaaatttacagaagcagtacgagaactggcagccaagggcccggtacaagcagagccttgacccaactgtggatgaagtcaagaagctctgcacgtccttacgtcgcaacgccaaggaggagcgagtcctctttcactacaatggccacggggtgccccggcccacagtcaacggggaggtctgggtcttcaacaagaactacacgcagtacatccctctgtccatatatgacctgcagacgtggatgggcagcccgtcgatcttcgtctacgactgctccaatgctggcttgatcgtcaagtccttcaagcagttcgcactacagcgggagcaggagctggaggtagctgcaatcaacccaaatcaccctcttgctcagatgcctttgcctccgtcgatgaaaaactgcatccagctggcagcctgcgaggccaccgagctgctgcccatgatccccgacctcccggctgacctattcacctcctgcctcaccacccccatcaagatcgccctgcgctggttttgcatgcagaaatgtgtcagtctggtgcctggcgtcacactggatttgatagaaaagatccctggccgcctgaacgacaggaggacgcccctgggtgaactgaactggatcttcacagccatcacagacaccatcgcgtggaacgtgctcccccgggatctcttccaaaagctcttcagacaggacttgctggtggctagtctgtttcgaaattttttattggcggaaaggattatgaggtcgtataactgcactcccgtcagcagcccgcgtctgccgcccacgtacatgcacgccatgtggcaagcctgggacctggctgttgacatctgtctgtctcagctgccgacgatcatcgaggaaggcactgcgtttcggcacagcccgttcttcgccgagcagctgaccgcattccaggtgtggctcaccatgggcgtggagaaccgaaacccacccgaacagctgcccatcgtcctgcaggtgctgttaagccaagtgcaccggctgagagcattggacttgcttggaagatttttggacctgggtccctgggcagtgagcctggccttgtctgtcggcatcttcccctacgtgctgaagctgctccagagctcggcccgagagctgcggccacttctcgttttcatctgggccaagatcctcgcagtggacagctcgtgccaagcggacctcgtgaaggacaacggccacaagtacttcctgtcggtcctggcggacccctacatgccagctgaacaccggaccatgacggctttcattctcgccgtgatcgtcaacagctatcacacggggcaggaagcctgccttcagggaaacctcattgccatctgcctggagcagctcaacgacccgcaccccttgctgcgccagtgggtggccatctgcctcggcaggatctggcagaacttcgactcggcgaggtggtgcggcgtgagggacagcgctcatgagaagctctacagcctcctctccgaccccattcccgaggtccgctgcgcagcggtcttcgcccttggcacgttcgtgggcaactctgcagagaggacggaccactccaccaccatcgaccacaacgtggccatgatgctggcccagctggtcagcgacgggagccccatggtccggaaggagctggtggtggctctgagtcatcttgtggttcagtatgaaagcaatttctgcaccgtggccctgcagttcatagaagaggaaaagaactacgccttgccttctccagcaaccacagagggagggagtttgaccccagtgcgagacagcccgtgcacccccagacttcgttctgtgagctcctatggaaacatccgtgctgtcgccacagccaggagcctcaacaaatctttgcagaacctgagtttgacagaggaatctggtggcgcggtggcgttctcccccggaaacctcagcaccagcagcagcgccagcagcaccctgggcagccccgagaatgaggagcatatcctgtccttcgagaccatcgacaagatgcgccgcgccagctcctactcctccctcaactccctcatcggagtttcctttaacagtgtttacactcagatttggagagtcctgctgcacctggctgctgacccctatccagaggtctcggacgtggccatgaaagtactcaacagcatcgcctacaaggccaccgtgaacgcccggccgcagcgcgtcctggacacctcctccctcacgcagtcggcccccgccagccccaccaacaagggcgtgcacatccaccaggcggggggctcccctccggcgtccagcaccagcagctccagcctgaccaacgatgtggccaagcagccggtcagccgagacttgccttctggccggccgggcaccacaggccccgctggggcgcagtacacccctcactcccaccagttcccccggacacggaagatgttcgacaagggcccagagcagactgcggacgacgcggacgatgctgctggacacaaaagtttcatctccgccacggtgcagacggggttctgcgactggagcgcccgctattttgcccagcccgtcatgaagatcccagaagagcacgacctggagagtcagatccgcaaggagcgggagtggcggttcctgcgaaacagccgtgtcaggaggcaggcccagcaagtcattcagaagggcattacgagattggacgaccaaatatttctgaacaggaaccccggcgtcccctctgtggtgaaattccaccccttcacgccgtgcatcgccgtagccgacaaggacagcatctgcttttgggactgggagaaaggggagaagctggattatttccacaatgggaaccctcggtacacgagggtcactgccatggagtatctgaacggccaggactgctcgcttctgctgacggccacagacgatggtgccatcagggtctggaagaattttgctgatttggaaaagaacccagagatggtgaccgcgtggcaggggctctcggacatgctgccaacgacgcgaggagctgggatggtggtggactgggagcaggagaccggcctcctcatgagctcaggagacgtgcggatcgtccggatctgggacacagaccgtgagatgaaggtgcaggacatccctacgggcgcagacagctgtgtgacgagtctgtcctgtgattcccaccgctcactcatcgtggctggcctcggtgacggctccatccgcgtctacgacagaaggatggcactcagcgaatgccgcgtcatgacgtaccgggagcacacagcctgggtggtgaaggcctccctgcagaagcgtcccgacggccacatcgtgagtgtgagcgtcaatggagatgtgcgcatctttgatccccggatgcctgagtcggtaaatgtgcttcagatcgtgaaggggctgacggccctggacatccacccccaggcggacctgatcgcatgtggctccgtcaatcagttcaccgccatctacaacagcagcggagagctcatcaacaacatcaagtactacgacggcttcatgggccagcgggtcggcgccatcagctgcctggccttccacccgcactggcctcacctggccgtgggaagcaacgactactacatctccgtgtactcggtggagaagcgtgtcagatag

SEQ ID NO: 43 - protein, 1335 aa, Homo sapiens
  Amino acid sequence of homo sapiens Raptor NM_020761.2
MESEMLQSPLLGLGEEDEADLTDWNLPLAFMKKRHCEKIEGSKSLAQSWRMKDRMKTVSVALVLCLNVGVDPPDVVKTTPCARLECWIDPLSMGPQKALETIGANLQKQYENWQPRARYKQSLDPTVDEVKKLCTSLRRNAKEERVLFHYNGHGVPRPTVNGEVWVFNKNYTQYIPLSIYDLQTWMGSPSIFVYDCSNAGLIVKSFKQFALQREQELEVAAINPNHPLAQMPLPPSMKNCIQLAACEATELLPMIPDLPADLFTSCLTTPIKIALRWFCMQKCVSLVPGVTLDLIEKIPGRLNDRRTPLGELNWIFTAITDTIAWNVLPRDLFQKLFRQDLLVASLFRNFLLAERIMRSYNCTPVSSPRLPPTYMHAMWQAWDLAVDICLSQLPTIIEEGTAFRHSPFFAEQLTAFQVWLTMGVENRNPPEQLPIVLQVLLSQVHRLRALDLLGRFLDLGPWAVSLALSVGIFPYVLKLLQSSARELRPLLVFIWAKILAVDSSCQADLVKDNGHKYFLSVLADPYMPAEHRTMTAFILAVIVNSYHTGQEACLQGNLIAICLEQLNDPHPLLRQWVAICLGRIWQNFDSARWCGVRDSAHEKLYSLLSDPIPEVRCAAVFALGTFVGNSAERTDHSTTIDHNVAMMLAQLVSDGSPMVRKELVVALSHLVVQYESNFCTVALQFIEEEKNYALPSPATTEGGSLTPVRDSPCTPRLRSVSSYGNIRAVATARSLNKSLQNLSLTEESGGAVAFSPGNLSTSSSASSTLGSPENEEHILSFETIDKMRRASSYSSLNSLIGVSFNSVYTQIWRVLLHLAADPYPEVSDVAMKVLNSIAYKATVNARPQRVLDTSSLTQSAPASPTNKGVHIHQAGGSPPASSTSSSSLTNDVAKQPVSRDLPSGRPGTTGPAGAQYTPHSHQFPRTRKMFDKGPEQTADDADDAAGHKSFISATVQTGFCDWSARYFAQPVMKIPEEHDLESQIRKEREWRFLRNSRVRRQAQQVIQKGITRLDDQIFLNRNPGVPSVVKFHPFTPCIAVADKDSICFWDWEKGEKLDYFHNGNPRYTRVTAMEYLNGQDCSLLLTATDDGAIRVWKNFADLEKNPEMVTAWQGLSDMLPTTRGAGMVVDWEQETGLLMSSGDVRIVRIWDTDREMKVQDIPTGADSCVTSLSCDSHRSLIVAGLGDGSIRVYDRRMALSECRVMTYREHTAWVVKASLQKRPDGHIVSVSVNGDVRIFDPRMPESVNVLQIVKGLTALDIHPQADLIACGSVNQFTAIYNSSGELINNIKYYDGFMGQRVGAISCLAFHPHWPHLAVGSNDYYISVYSVEKRVR

SEQ ID NO: 44 - RNA, 2033 nt, Homo sapiens
  Nucleotide sequence encoding homo sapiens PRAS40 NM_001098632.1
auauuguauacuggaauugaagccaaggagguaccauuuugcucgagggcauggccuaagccggucagcuaaggccauguuaauacggggcugucccaucucucugcggggcgcgacagcuggaagagccgaacggauaagagaagaggaggugagaggagcuguacaccacaagaggcacugagggacucaggauaacgggaugaagccgucagugcccccagaaacgaagcggccccggacgaauuucugagucaccgucgcgagaaagcgggcugagccgccauuuugaagccuggcaaaccgaagcaagaaaugcugccguguuggaucuuugccagccuucgugccgaaugggagcagggcgcggauggcgucggggcgccccgaggagcugugggaggccguggugggggccgcugagcgcuuccgggcccggacuggcacggagcuggugcugcugaccgcggccccgccgccaccaccccgcccgggccccugugccuaugcugcccauggucgaggagcccuggcggaggcagcgcgccguugccuccacgacaucgcacuggcccacagggcugccacugcugcucggccuccugcgcccccaccagcaccacagccacccagucccacacccagcccaccccggccuacccuggccagagaggacaacgaggaggacgaggaugagcccacagagacagagaccuccggggagcagcugggcauuagugauaauggagggcucuuugugauggaugaggacgccacccuccaggaccuuccccccuucugugagucagaccccgagaguacagaugauggcagccugagcgaggagacccccgccggcccccccaccugcucagugcccccagccucagcccuacccacacagcaguacgccaagucccugccugugucugugcccgucuggggcuucaaggagaagaggacagaggcgcggucaucagaugaggagaaugggccgcccucuucgcccgaccuggaccgcaucgcggcgagcaugcgcgcgcuggugcugcgagaggccgaggacacccaggucuucggggaccugccacggccgcggcuuaacaccagcgacuuccagaagcugaagcggaaauauugaaguccagggagggagcgccccgggccgcguccgccccgucccacacuacgcccccgccccacucccggggccugcuaaucugaggccgauccgggaccggccuccuugcgucucccauucccaagauugucccgccucugccaauccccgccguccuuccagcccacgaccugccgcgccgaggagcggcaucugucccguuucccgauugggucugucgucucucuccgccuagcgacagauuccuucuauuaagggauuggcucgcugaguucuaagcucuaaaugggucaacuccuuuguuuuccgccuagcgacaagggauuugcucgcacggcauuggcuccauccccuagucgcuggacagcucuuuuuuugauuggcucaaauccuguaaagggcuugaccagucucuacauagucaccguccgcuuuuccugaguucucccucccaauuggcuccagcuuccugggggcguggccaagcccuccucuucccagaauuggcccggggccuucaauuuacguucuuuacacuacggggacuggggucgucuuugcccacgucccgacaacuuguucccugacccccucagggauggccccaaacugucccugccucuggcacccccuuucauugguuccauccauccccacaacagccugccaaucgaagcccgucccugcauccaggaugguaccagcucccgccccucgccccccaccuccacaggugccuuaaagggcccucguccacccaagguggggggcaggggcccucacucuccggcccuggugugggggagagagugagggguugggggaucggcaguugggaggggcgcucugagauuaaagaguuuuaccucugagauaaaaaaaaaaaaaaaaa

SEQ ID NO: 45 - DNA, 771 bp, Homo sapiens
  Nucleotide sequence encoding homo sapiens PRAS40 NM_001098632.1
atggcgtcggggcgccccgaggagctgtgggaggccgtggtgggggccgctgagcgcttccgggcccggactggcacggagctggtgctgctgaccgcggccccgccgccaccaccccgcccgggcccctgtgcctatgctgcccatggtcgaggagccctggcggaggcagcgcgccgttgcctccacgacatcgcactggcccacagggctgccactgctgctcggcctcctgcgcccccaccagcaccacagccacccagtcccacacccagcccaccccggcctaccctggccagagaggacaacgaggaggacgaggatgagcccacagagacagagacctccggggagcagctgggcattagtgataatggagggctctttgtgatggatgaggacgccaccctccaggaccttccccccttctgtgagtcagaccccgagagtacagatgatggcagcctgagcgaggagacccccgccggcccccccacctgctcagtgcccccagcctcagccctacccacacagcagtacgccaagtccctgcctgtgtctgtgcccgtctggggcttcaaggagaagaggacagaggcgcggtcatcagatgaggagaatgggccgccctcttcgcccgacctggaccgcatcgcggcgagcatgcgcgcgctggtgctgcgagaggccgaggacacccaggtcttcggggacctgccacggccgcggcttaacaccagcgacttccagaagctgaagcggaaatattga

SEQ ID NO: 46 - protein, 256 aa, Homo sapiens
  Amino acid sequence of homo sapiens PRAS40 NM_001098632.1
MASGRPEELWEAVVGAAERFRARTGTELVLLTAAPPPPPRPGPCAYAAHGRGALAEAARRCLHDIALAHRAATAARPPAPPPAPQPPSPTPSPPRPTLAREDNEEDEDEPTETETSGEQLGISDNGGLFVMDEDATLQDLPPFCESDPESTDDGSLSEETPAGPPTCSVPPASALPTQQYAKSLPVSVPVWGFKEKRTEARSSDEENGPPSSPDLDRIAASMRALVLREAEDTQVFGDLPRPRLNTSDFQKLKRKY

SEQ ID NO: 47 - RNA, 1821 nt, Homo sapiens
  Nucleotide sequence encoding homo sapiens PRAS40 NM_001098633.2
auauuguauacuggaauugaagccaaggagguaccauuuugcucgagggcauggccuaagccggucagcuaaggccauguuaauacggggcugucccaucucucugcggggcgcgacagcuggaagagccgaacggauaagagaagaggagggcgcggauggcgucggggcgccccgaggagcugugggaggccguggugggggccgcugagcgcuuccgggcccggacuggcacggagcuggugcugcugaccgcggccccgccgccaccaccccgcccgggccccugugccuaugcugcccauggucgaggagcccuggcggaggcagcgcgccguugccuccacgacaucgcacuggcccacagggcugccacugcugcucggccuccugcgcccccaccagcaccacagccacccagucccacacccagcccaccccggccuacccuggccagagaggacaacgaggaggacgaggaugagcccacagagacagagaccuccggggagcagcugggcauuagugauaauggagggcucuuugugauggaugaggacgccacccuccaggaccuuccccccuucugugagucagaccccgagaguacagaugauggcagccugagcgaggagacccccgccggcccccccaccugcucagugcccccagccucagcccuacccacacagcaguacgccaagucccugccugugucugugcccgucuggggcuucaaggagaagaggacagaggcgcggucaucagaugaggagaaugggccgcccucuucgcccgaccuggaccgcaucgcggcgagcaugcgcgcgcuggugcugcgagaggccgaggacacccaggucuucggggaccugccacggccgcggcuuaacaccagcgacuuccagaagcugaagcggaaauauugaaguccagggagggagcgccccgggccgcguccgccccgucccacacuacgcccccgccccacucccggggccugcuaaucugaggccgauccgggaccggccuccuugcgucucccauucccaagauugucccgccucugccaauccccgccguccuuccagcccacgaccugccgcgccgaggagcggcaucugucccguuucccgauugggucugucgucucucuccgccuagcgacagauuccuucuauuaagggauuggcucgcugaguucuaagcucuaaaugggucaacuccuuuguuuuccgccuagcgacaagggauuugcucgcacggcauuggcuccauccccuagucgcuggacagcucuuuuuuugauuggcucaaauccuguaaagggcuugaccagucucuacauagucaccguccgcuuuuccugaguucucccucccaauuggcuccagcuuccugggggcguggccaagcccuccucuucccagaauuggcccggggccuucaauuuacguucuuuacacuacggggacuggggucgucuuugcccacgucccgacaacuuguucccugacccccucagggauggccccaaacugucccugccucuggcacccccuuucauugguuccauccauccccacaacagccugccaaucgaagcccgucccugcauccaggaugguaccagcucccgccccucgccccccaccuccacaggugccuuaaagggcccucguccacccaagguggggggcaggggcccucacucuccggcccuggugugggggagagagugagggguugggggaucggcaguugggaggggcgcucugagauuaaagaguuuuaccucugagauaaaaaaaaaaaaaaaaa

SEQ ID NO: 48 - DNA, 771 bp, Homo sapiens; identical to SEQ ID NO: 45
  Nucleotide sequence encoding homo sapiens PRAS40 NM_001098633.2

SEQ ID NO: 49 - protein, 256 aa, Homo sapiens; identical to SEQ ID NO: 46
  Amino acid sequence of homo sapiens PRAS40 NM_001098633.2

SEQ ID NO: 50 - RNA, 2455 nt, Homo sapiens
  Nucleotide sequence encoding homo sapiens PRAS40 NM_032375.4
auauuguauacuggaauugaagccaaggagguaccauuuugcucgagggcauggccuaagccggucagcuaaggccauguuaauacggggcugucccaucucucugcggggcgcgacagcuggaagagccgaacggauaagagaagaggaggugagaggagcuguacaccacaagaggcacugagggacucaggauaacgggaugaagccgucagugcccccagaaacgaagcggccccggacgaauuucugagucaccgucgcgagaaagcgggcugagccgccauuuugaagccuggcaaaccgaagcaagaaaugcugccguguuggaucuuugccagccuucgugccgaaugggagcagguuggagggagggagagccaauauacacuaugggcugauuaagcccgguuggcugccauguuguuaacgagcaccgauuuccucuacuuuugucgaagaaguuuauugugggucagggacgucaggucgcuugccuucguuuacuguggucaugauugagcauaugaggacggccauuauuguugggggcaaauggaaaugcucuaggcggggccauuuuucuuaggggcaagcugucgucacccuugucaacugguucggaugaagccccuguggccgccaucuugaucucgggcggccccgauaagggaggcggagugugcggagaggaggcggggcaacugcgcggacgugacgcaaggcgccgccaugucuuuugagggcggugacggcgccgggccggccaugcuggcuacgggcacggcgcggauggcgucggggcgccccgaggagcugugggaggccguggugggggccgcugagcgcuuccgggcccggacuggcacggagcuggugcugcugaccgcggccccgccgccaccaccccgcccgggccccugugccuaugcugcccauggucgaggagcccuggcggaggcagcgcgccguugccuccacgacaucgcacuggcccacagggcugccacugcugcucggccuccugcgcccccaccagcaccacagccacccagucccacacccagcccaccccggccuacccuggccagagaggacaacgaggaggacgaggaugagcccacagagacagagaccuccggggagcagcugggcauuagugauaauggagggcucuuugugauggaugaggacgccacccuccaggaccuuccccccuucugugagucagaccccgagaguacagaugauggcagccugagcgaggagacccccgccggcccccccaccugcucagugcccccagccucagcccuacccacacagcaguacgccaagucccugccugugucugugcccgucuggggcuucaaggagaagaggacagaggcgcggucaucagaugaggagaaugggccgcccucuucgcccgaccuggaccgcaucgcggcgagcaugcgcgcgcuggugcugcgagaggccgaggacacccaggucuucggggaccugccacggccgcggcuuaacaccagcgacuuccagaagcugaagcggaaauauugaaguccagggagggagcgccccgggccgcguccgccccgucccacacuacgcccccgccccacucccggggccugcuaaucugaggccgauccgggaccggccuccuugcgucucccauucccaagauugucccgccucugccaauccccgccguccuuccagcccacgaccugccgcgccgaggagcggcaucugucccguuucccgauugggucugucgucucucuccgccuagcgacagauuccuucuauuaagggauuggcucgcugaguucuaagcucuaaaugggucaacuccuuuguuuuccgccuagcgacaagggauuugcucgcacggcauuggcuccauccccuagucgcuggacagcucuuuuuuugauuggcucaaauccuguaaagggcuugaccagucucuacauagucaccguccgcuuuuccugaguucucccucccaauuggcuccagcuuccugggggcguggccaagcccuccucuucccagaauuggcccggggccuucaauuuacguucuuuacacuacggggacuggggucgucuuugcccacgucccgacaacuuguucccugacccccucagggauggccccaaacugucccugccucuggcacccccuuucauugguuccauccauccccacaacagccugccaaucgaagcccgucccugcauccaggaugguaccagcucccgccccucgccccccaccuccacaggugccuuaaagggcccucguccacccaagguggggggcaggggcccucacucuccggcccuggugugggggagagagugagggguugggggaucggcaguugggaggggcgcucugagauuaaagaguuuuaccucugagauaaaaaaaaaaaaaaaaa

SEQ ID NO: 51 - DNA, 831 bp, Homo sapiens
  Nucleotide sequence encoding homo sapiens PRAS40 NM_032375.4
atgtcttttgagggcggtgacggcgccgggccggccatgctggctacgggcacggcgcggatggcgtcggggcgccccgaggagctgtgggaggccgtggtgggggccgctgagcgcttccgggcccggactggcacggagctggtgctgctgaccgcggccccgccgccaccaccccgcccgggcccctgtgcctatgctgcccatggtcgaggagccctggcggaggcagcgcgccgttgcctccacgacatcgcactggcccacagggctgccactgctgctcggcctcctgcgcccccaccagcaccacagccacccagtcccacacccagcccaccccggcctaccctggccagagaggacaacgaggaggacgaggatgagcccacagagacagagacctccggggagcagctgggcattagtgataatggagggctctttgtgatggatgaggacgccaccctccaggaccttccccccttctgtgagtcagaccccgagagtacagatgatggcagcctgagcgaggagacccccgccggcccccccacctgctcagtgcccccagcctcagccctacccacacagcagtacgccaagtccctgcctgtgtctgtgcccgtctggggcttcaaggagaagaggacagaggcgcggtcatcagatgaggagaatgggccgccctcttcgcccgacctggaccgcatcgcggcgagcatgcgcgcgctggtgctgcgagaggccgaggacacccaggtcttcggggacctgccacggccgcggcttaacaccagcgacttccagaagctgaagcggaaatattga

SEQ ID NO: 52 - protein, 276 aa, Homo sapiens
  Amino acid sequence of homo sapiens PRAS40 NM_032375.4
MSFEGGDGAGPAMLATGTARMASGRPEELWEAVVGAAERFRARTGTELVLLTAAPPPPPRPGPCAYAAHGRGALAEAARRCLHDIALAHRAATAARPPAPPPAPQPPSPTPSPPRPTLAREDNEEDEDEPTETETSGEQLGISDNGGLFVMDEDATLQDLPPFCESDPESTDDGSLSEETPAGPPTCSVPPASALPTQQYAKSLPVSVPVWGFKEKRTEARSSDEENGPPSSPDLDRIAASMRALVLREAEDTQVFGDLPRPRLNTSDFQKLKRKY

SEQ ID NO: 53 - DNA, 144 bp, Homo sapiens
  N-terminal target region of Isoform 2 of SLC38A9
atgaacaagagaattcattactacagccggctcaccactcctgcagacaaggcactgattgccccagaccatgtagttccagctccagaagagtgctatgtgtatagtccattgggctctgcttataaacttcaaagttacact

SEQ ID NO: 54 - protein, 48 aa, Homo sapiens
  N-terminal target region of Isoform 2 of SLC38A9
MNKRIHYYSRLTTPADKALIAPDHVVPAPEECYVYSPLGSAYKLQSYT

SEQ ID NO: 55 - DNA, 252 bp, Homo sapiens
  N-terminal target region of Isoform 3 of SLC38A9
atggctatttgcattttaacatggagaatccggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacact

SEQ ID NO: 56 - protein, 84 aa, Homo sapiens
  N-terminal target region of Isoform 3 of SLC38A9
MAICILTWRIRPFCIEPTNIVNVNHVIQRVSDHASAMNKRIHYYSRLTTPADKALIAPDHVVPAPEECYVYSPLGSAYKLQSYT

SEQ ID NO: 57 - DNA, 243 bp, Homo sapiens
  Target region of Isoform 1 of SLC38A9
ccatcggatctaagatccaaaaggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacact

SEQ ID NO: 58 - protein, 81 aa, Homo sapiens
  Target region of Isoform 1 of SLC38A9
PSDLRSKRPFCIEPTNIVNVNHVIQRVSDHASAMNKRIHYYSRLTTPADKALIAPDHVVPAPEECYVYSPLGSAYKLQSYT

SEQ ID NO: 59 - DNA, 144 bp, Homo sapiens; identical to SEQ ID NO: 53
  Target region of Isoform 2 of SLC38A9

SEQ ID NO: 60 - protein, 48 aa, Homo sapiens; identical to SEQ ID NO: 54
  Target region of Isoform 2 of SLC38A9

SEQ ID NO: 61 - DNA, 222 bp, Homo sapiens
  Target region of Isoform 3 of SLC38A9
cggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacact

SEQ ID NO: 62 - protein, 74 aa, Homo sapiens
  Target region of Isoform 3 of SLC38A9
RPFCIEPTNIVNVNHVIQRVSDHASAMNKRIHYYSRLTTPADKALIAPDHVVPAPEECYVYSPLGSAYKLQSYT

SEQ ID NO: 63 - RNA, 19 nt, Homo sapiens
  target nt 530-548 of human SLC38A9 mRNA (NM_173514.3)
acacugaaggauacgguaa

SEQ ID NO: 64 - RNA, 19 nt, Homo sapiens
  target nt 262-280 of human SLC38A9 mRNA (NM_173514.3)
gauccuggaccuaugaaua

SEQ ID NO: 65 - RNA, 19 nt, Homo sapiens
  target nt 478-496 of human SLC38A9 mRNA (NM_173514.3)
gaagagugcuauguguaua

SEQ ID NO: 66 - RNA, 19 nt, Homo sapiens
  target nt 355-373 of human SLC38A9 mRNA (NM_173514.3)
caugucauucagaggguua

SEQ ID NO: 67 - DNA, 1350 bp, Homo sapiens
ggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctaggcacagtgtctgtcctttatttgattttccttgtcacctttaaggctgttcgcttgggatttcatttggaatttcattggtttataccaacagaattttttgtaccagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtga

SEQ ID NO: 68 - protein, 449 aa, Homo sapiens
GYGKNTSLVTIFMIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILGTVSVLYLIFLVTFKAVRLGFHLEFHWFIPTEFFVPEIRFQFPQLTGVLTLAFFIHNCIITLLKNNKKQENNVRDLCIAYMLVTLTYLYIGVLVFASFPSPPLSKDCIEQNFLDNFPSSDTLSFIARIFLLFQMMTVYPLLGYLARVQLLGHIFGDIYPSIFHVLILNLIIVGAGVIMACFYPNIGGIIRYSGAACGLAFVFIYPSLIYIISLHQEERLTWPKLIFHVFIIILGVANLIVQFFM

SEQ ID NO: 69 - DNA, 1350 bp, Homo sapiens; identical to SEQ ID NO: 67

SEQ ID NO: 70 - protein, 449 aa, Homo sapiens; identical to SEQ ID NO: 68

SEQ ID NO: 71 - DNA, 1245 bp, Homo sapiens
gaaggatacggtaaaaacaccagtttagtaaccatttttatgatttggaataccatgatgggaacatctatactaagcattccttggggcataaaacaggctggatttactactggaatgtgtgtcatcatactgatgggccttttaacactttattgctgctacagagtagtgaaatcacggactatgatgttttcgttggataccactagctgggaatatccagatgtctgcagacattatttcggctcctttgggcagtggtcgagtctccttttctccttggtgtctctcattggagcaatgatagtttattgggtgcttatgtcaaattttctttttaatactggaaagtttatttttaattttattcatcacattaatgacacagacactatactgagtaccaataatagcaaccctgtgatttgtccaagtgccgggagtggaggccatcctgacaacagctctatgattttctatgccaatgacacaggagcccaacagtttgaaaagtggtgggataagtccaggacagtccccttttatcttgtagggctcctcctcccactgctcaatttcaagtctccttcatttttttcaaaatttaatatcctagagataagatttcagtttccacagctgactggagtgcttacccttgctttttttattcataattgtatcatcacactcttgaagaacaacaagaaacaagaaaacaatgtgagggacttgtgcattgcttatatgctggtgacattaacttatctctatattggagtcctggtttttgcttcatttccttcaccaccattatccaaagattgtattgagcagaattttttagacaacttccctagcagtgacaccctgtccttcattgcaaggatattcctgctgttccagatgatgactgtatacccactcttaggctacctggctcgtgtccagcttttgggccatatcttcggtgacatttatcctagcattttccatgtgctgattcttaatctaattattgtgggagctggagtgatcatggcctgtttctacccaaacataggagggatcataagatattcaggagcagcatgtggactggcctttgtattcatatacccatctctcatctatataatttccctccaccaagaagagcgtctgacatggcctaaattaatcttccacgttttcatcatcattttgggcgtggctaacctgattgttcagttttttatgtga

SEQ ID NO: 72 - protein, 414 aa, Homo sapiens
EGYGKNTSLVTIFMIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILEIRFQFPQLTGVLTLAFFIHNCIITLLKNNKKQENNVRDLCIAYMLVTLTYLYIGVLVFASFPSPPLSKDCIEQNFLDNFPSSDTLSFIARIFLLFQMMTVYPLLGYLARVQLLGHIFGDIYPSIFHVLILNLIIVGAGVIMACFYPNIGGIIRYSGAACGLAFVFIYPSLIYIISLHQEERLTWPKLIFHVFIIILGVANLIVQFFM

SEQ ID NO: 73 - DNA, 1314 bp, Homo sapiens; identical to SEQ ID NO: 18

SEQ ID NO: 74 - protein, 437 aa, Homo sapiens
MIWNTMMGTSILSIPWGIKQAGFTTGMCVIILMGLLTLYCCYRVVKSRTMMFSLDTTSWEYPDVCRHYFGSFGQWSSLLFSLVSLIGAMIVYWVLMSNFLFNTGKFIFNFIHHINDTDTILSTNNSNPVICPSAGSGGHPDNSSMIFYANDTGAQQFEKWWDKSRTVPFYLVGLLLPLLNFKSPSFFSKFNILGTVSVLYLIFLVTFKAVRLGFHLEFHWFIPTEFFVPEIRFQFPQLTGVLTLAFFIHNCIITLLKNNKKQENNVRDLCIAYMLVTLTYLYIGVLVFASFPSPPLSKDCIEQNFLDNFPSSDTLSFIARIFLLFQMMTVYPLLGYLARVQLLGHIFGDIYPSIFHVLILNLIIVGAGVIMACFYPNIGGIIRYSGAACGLAFVFIYPSLIYIISLHQEERLTWPKLIFHVFIIILGVANLIVQFFM

SEQ ID NO: 75 - protein, 4 aa, Homo sapiens
YYSR

SEQ ID NO: 76 - DNA, 12 bp, Homo sapiens
tactacagccgg

SEQ ID NO: 77 - RNA, 19 nt, Homo sapiens
acacugaaggauacgguaa

SEQ ID NO: 78 - RNA, 19 nt, Homo sapiens
gauccuggaccuaugaaua

SEQ ID NO: 79 - RNA, 19 nt, Homo sapiens
gaagagugcuauguguaua

SEQ ID NO: 80 - RNA, 19 nt, Homo sapiens
caugucauucagaggguua

SEQ ID NO: 81 - RNA, 19 nt, Homo sapiens
ucuccaggauagcugcuua

SEQ ID NO: 82 - RNA, 19 nt, Homo sapiens
ggcuuauacaguacccuaa

SEQ ID NO: 83 - RNA, 19 nt, Homo sapiens
aagugaggguagaaccuuu

SEQ ID NO: 84 - RNA, 19 nt, Homo sapiens
guuugucacccucgauaaa

SEQ ID NO: 85 - RNA, 20 nt, Artificial Sequence
  primer SLC38A9
tcctttgggcagtggtcgag

SEQ ID NO: 86 - RNA, 20 nt, Artificial Sequence
  primer SLC38A9
actcccggcacttggacaaa

SEQ ID NO: 87 - RNA, 18 nt, Artificial Sequence
  primer GAPDH
gaaggtgaaggtcggagt

SEQ ID NO: 88 - RNA, 20 nt, Artificial Sequence
  primer GAPDH
gaagatggtgatgggatttc

SEQ ID NO: 89 - protein, 561 aa, Homo sapiens; identical to SEQ ID NO: 3
  LAMTORs_PD - SLC 38A9 Peptide Mapping

SEQ ID NO: 90 - protein, 561 aa, Homo sapiens; identical to SEQ ID NO: 3
  SLC38A9_PD - SLC38A9 Peptide Mapping

SEQ ID NO: 91 - protein, 111 aa, Homo sapiens; identical to SEQ ID NO: 22
  Sequence alignment of the N-terminal cytoplasmic region of homo sapiens SLC38A9

SEQ ID NO: 92 - protein, 110 aa, Mus musculus
  Sequence alignment of the N-terminal cytoplasmic region of mouse SLC38A9
MASVDGDSRHLLSEVEHEVSPGPMNIQFDSSDLRSKRPFYIEPTNIVNVNDVIQRVSDHAAAMNKRIHYYSRLTTPADKALIAPDHVVPAPEECYVYSPLGSAYKLKSYT

SEQ ID NO: 93 - protein, 110 aa, Rattus
  Sequence alignment of the N-terminal cytoplasmic region of rat SLC38A9
MANVDSDSRHLISEVEHEVNPGPMNIQFDSSDLRSKRPFYIEPTNIVNVNDVIQKVSDHAAAMNKRIHYYSRLTTPADKALIAPDHVVPAPEECYVYSPLGSAYKLKSYT

SEQ ID NO: 94 - protein, 104 aa, Xenopus <genus>
  Sequence alignment of the N-terminal cytoplasmic region of Xenopus SLC38A9
MDSDQTPLINPSLFEECAQNHFAATDPRSRRPFHIEPSYITSINDDDPQRITSVASAMNKRIHYYSKLSNPSDKGLIAPDHVLPAPEEIYVYSPLGTALKIDGS

SEQ ID NO: 95 - protein, 99 aa, Danio rerio
  Sequence alignment of the N-terminal cytoplasmic region of zebrafish SLC38A9
MDEDSKPLLGSVPTGDYYTDSLDPKQRRPFHVEPRNIVGEDVQERVSAEAAVLSSRVHYYSRLTGSSDRLLAPPDHVIPSHEDIYIYSPLGTAFKVQGG

SEQ ID NO: 96 - DNA, 336 bp, Homo sapiens
  Nucleotide sequence encoding the N-terminal cytoplasmic region of homo sapiens SLC38A9 (Sentor) isoform 1
atggcaaatatgaatagtgattctaggcatcttggcacctctgaggtagatcatgaaagagatcctggacctatgaatatccagtttgagccatcggatctaagatccaaaaggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaa

SEQ ID NO: 97 - protein, 112 aa, Homo sapiens
  Amino acid sequence of the N-terminal cytoplasmic region of homo sapiens SLC38A9 (Sentor) isoform 1
MANMNSDSRHLGTSEVDHERDPGPMNIQFEPSDLRSKRPFCIEPTNIVNVNHVIQRVSDHASAMNKRIHYYSRLTTPADKALIAPDHVVPAPEECYVYSPLGSAYKLQSYTE

SEQ ID NO: 98 - DNA, 147 bp, Homo sapiens
  N-terminal target region of Isoform 2 of SLC38A9
atgaacaagagaattcattactacagccggctcaccactcctgcagacaaggcactgattgccccagaccatgtagttccagctccagaagagtgctatgtgtatagtccattgggctctgcttataaacttcaaagttacactgaa

SEQ ID NO: 99 - protein, 49 aa, Homo sapiens
  N-terminal target region of Isoform 2 of SLC38A9
MNKRIHYYSRLTTPADKALIAPDHVVPAPEECYVYSPLGSAYKLQSYTE

SEQ ID NO: 100 - DNA, 255 bp, Homo sapiens
  N-terminal target region of Isoform 3 of SLC38A9
atggctatttgcattttaacatggagaatccggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaa

SEQ ID NO: 101 - protein, 85 aa, Homo sapiens
  N-terminal target region of Isoform 3 of SLC38A9
MAICILTWRIRPFCIEPTNIVNVNHVIQRVSDHASAMNKRIHYYSRLTTPADKALIAPDHVVPAPEECYVYSPLGSAYKLQSYTE

SEQ ID NO: 102 - DNA, 246 bp, Homo sapiens
  Target region of Isoform 1 of SLC38A9
ccatcggatctaagatccaaaaggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaa

SEQ ID NO: 103 - protein, 82 aa, Homo sapiens
  Target region of Isoform 1 of SLC38A9
PSDLRSKRPFCIEPTNIVNVNHVIQRVSDHASAMNKRIHYYSRLTTPADKALIAPDHVVPAPEECYVYSPLGSAYKLQSYTE

SEQ ID NO: 104 - DNA, 147 bp, Homo sapiens; identical to SEQ ID NO: 98
  Target region of Isoform 2 of SLC38A9

SEQ ID NO: 105 - protein, 49 aa, Homo sapiens; identical to SEQ ID NO: 99
  Target region of Isoform 2 of SLC38A9

SEQ ID NO: 106 - DNA, 225 bp, Homo sapiens
  Target region of Isoform 3 of SLC38A9
cggcctttctgtatagagcccacaaacatcgtgaatgtgaatcatgtcattcagagggttagtgaccatgcctctgccatgaacaagagaattcattactacagccggctcaccactcctgcagacaaggcactgattgccccagaccatgtagttccagctccagaagagtgctatgtgtatagtccattgggctctgcttataaacttcaaagttacactgaa

SEQ ID NO: 107 - protein, 75 aa, Homo sapiens
  Target form of Isoform 3 of SLC38A9
RPFCIEPTNIVNVNHVIQRVSDHASAMNKRIHYYSRLTTPADKALIAPDHVVPAPEECYVYSPLGSAYKLQSYTE

SEQ ID NO: 108 - protein, 112 aa, Homo sapiens; identical to SEQ ID NO: 97
  Sequence alignment of the N-terminal cytoplasmic region of homo sapiens SLC38A9

SEQ ID NO: 109 - protein, 111 aa, Mus musculus
  Sequence alignment of the N-terminal cytoplasmic region of mouse SLC38A9
MASVDGDSRHLLSEVEHEVSPGPMNIQFDSSDLRSKRPFYIEPTNIVNVNDVIQRVSDHAAAMNKRIHYYSRLTTPADKALIAPDHVVPAPEECYVYSPLGSAYKLKSYTE

SEQ ID NO: 110 - protein, 111 aa, Rattus
  Sequence alignment of the N-terminal cytoplasmic region of rat SLC38A9
MANVDSDSRHLISEVEHEVNPGPMNIQFDSSDLRSKRPFYIEPTNIVNVNDVIQKVSDHAAAMNKRIHYYSRLTTPADKALIAPDHVVPAPEECYVYSPLGSAYKLKSYTE

SEQ ID NO: 111 - protein, 105 aa, Xenopus <genus>
  Sequence alignment of the N-terminal cytoplasmic region of Xenopus SLC38A9
MDSDQTPLINPSLFEECAQNHFAATDPRSRRPFHIEPSYITSINDDDPQRITSVASAMNKRIHYYSKLSNPSDKGLIAPDHVLPAPEEIYVYSPLGTALKIDGSD

SEQ ID NO: 112 - protein, 100 aa, Danio rerio
  Sequence alignment of the N-terminal cytoplasmic region of zebrafish SLC38A9
MDEDSKPLLGSVPTGDYYTDSLDPKQRRPFHVEPRNIVGEDVQERVSAEAAVLSSRVHYYSRLTGSSDRLLAPPDHVIPSHEDIYIYSPLGTAFKVQGGD

SEQ ID NO: 113 - DNA, 59 bp, Artificial Sequence
  DNA sequence corresponding to shRNA sequence
ccgggccttgacaacagttctatatctcgagatatagaactgttgtcaaggcttttttg

SEQ ID NO: 114 - DNA, 21 bp, Artificial Sequence
  DNA sequence corresponding to shRNA sequence
atatagaactgttgtcaaggc